(19) United States
(12) Patent Application Publication
(10) Pub. No.: US 2009/0203663 A1
(43) Pub. Date: Aug. 13, 2009

(54) CHEMICAL COMPOUNDS

(75) Inventors: Jean Claude Arnould, Reims (FR); Benedicte Delouvrie, Reims (FR); Jason Grant Kettle, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/278,452

(22) PCT Filed: Feb. 7, 2007
(86) PCT No.: PCT/GB07/00404

§ 371 Date: Aug. 6, 2008

(30) Foreign Application Priority Data
Feb. 9, 2006 (EP) 06300120.0

(51) Int. Cl. A61K 31/397; C07C 229/36; A61K 31/216; C07D 205/04; C07D 211/46; A61K 31/445; A61P 35/00

(52) U.S. Cl. 514/210.01; 560/39; 514/539; 548/952; 546/221; 514/327

(57) ABSTRACT

The present invention relates to compounds that inhibit a5b1 function, processes for their preparation, pharmaceutical compositions containing them as the active ingredient, to their use as medicaments and to their use in the manufacture of medicaments for use in the treatment in warm blooded animals such as humans of diseases that have a significant angiogenesis or vascular component such as for treatment of solid tumours. The present invention also relates to compounds that inhibit a5b1, and also that exhibit appropriate selectivity profile(s) against other integrins.

The present invention relates to chemical compounds useful as pharmaceuticals in particular in the treatment of diseases in which a5b1 function is a factor, to process for their preparation, and to compositions containing these as well as their use in therapy.

### BACKGROUND OF THE INVENTION

Many normal physiological and disease processes require cells to contact other cells and/or extracellular matrix. Cell-matrix and cell-cell adhesion is mediated through several families of proteins including integrins, selecting, cadherins, and immunoglobulins, and facilitates a variety of normal cellular functions such as proliferation, migration, differentiation or survival. Cell adhesion is also key to a range of pathologies, and so pharmacological disruption of cell adhesion interactions can provide a mechanism for therapeutic intervention. In particular members of the integrin superfamily of adhesion molecules are believed to play a particularly important role in acute and chronic disease states such as cancer, inflammatory diseases, stroke and neurodegenerative disorders. Thus, integrins represent a very complex biological area.

The integrin superfamily of cell surface receptors is formed from a number of structurally and functionally related surface glycoproteins, with each receptor existing as a heterodimer of non-covalently linked α and β subunits. To date, at least 18 different α and 8 β subunits have been identified in mammals, which are known to form more than 24 different receptors. Each integrin interacts specifically with defined extracellular ligands, including extracellular matrix proteins such as, fibronectin, fibrinogen, vitronectin, collagen and cell surface molecules such as VCAM, ICAM and PECAM, via linear adhesion motifs.

The integrin α5β1 (hereinafter a5b1) is composed of an α5 (hereinafter a5) and β1 (hereinafter b1) subunits, the a5 subunit forming a specific dimer with the b1 subunit, and is widely expressed in most tissues. Integrin a5b1 almost exclusively mediates cell adhesion through an interaction with fibronectin, binding via the short arginine-glycine-aspartate (RGD) adhesion motif. Endothelial cells can however bind to fibrin via a5b1. There is compelling evidence that the a5b1 interaction with fibronectin plays an important role in physiopathological angiogenesis and vascular integrity. Although endothelial cells express a variety of integrins, a5b1 is important for survival of endothelial cells on provisional matrix in vitro, suppressing apoptosis and promoting proliferation. Furthermore, immunohistochemical analysis, and imaging have both shown that a5b1 expression is upregulated in tumour vasculature. Consistent with a key functional role for the receptor-ligand pairing, the a5b1 ligand fibronectin is also upregulated in tumour tissue and during wound-healing. Transgenic studies further support an important role for a5b1 in the vasculature. Both a5 and b1 knock-out mice are embryonic lethal and display defects in development of early vascular systems, suggesting a pivotal functional role in early vasculogenesis. Moreover, studies using agents such as blocking RGD peptides or neutralising antibodies have shown that disruption of a5b1 interaction with its cognate ligands has anti-angiogenic effects in vivo. As well as inhibiting angiogenesis, a5b1 inhibitors may reduce the proliferation of certain tumour cells that express the receptor.

In addition to a5b1, other integrin family members such as avb3 and aiibb3 can also interact with RGD-containing ligands. Other integrins can bind to ligands via non-RGD binding domains. An example of particular importance and relevance is a4b1 which binds via a leucine-aspartate-valine (LDV) motif to ligands that include the connecting segment-1 region of fibronectin, VCAM-1, MAdCAM or to the SVVYGLR motif found within osteopontin.

Since there are a variety of integrins that share the same ligand or binding-domain with a5b1, it will be important to develop therapeutic agents that are selective towards a5b1 activity. However, as other endothelial integrins such as avb3, avb5 and a4b1 are also involved in possible pathological events, agents which target such integrins in addition to a5b1, may have additional therapeutic activity.

Taken together, the expression and functional data suggest that selective inhibition of a5b1 function provides an attractive therapeutic strategy to combat diseases that have a significant angiogenesis or vascular component such as for treatment of solid tumours. There is thus a clear need to develop compounds that inhibit a5b1 with appropriate pharmacokinetic and pharmacodynamic drug properties, and also that exhibit appropriate selectivity profile(s) against other integrins.

### SUMMARY OF THE INVENTION

These and other needs are met by compounds falling within the present invention. The present invention provides a compound of formula I:

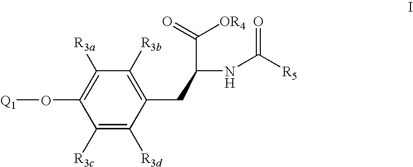

or a pharmaceutical acceptable salt thereof, wherein:

Q₁ is:

(i) a group of sub-formula (ai)

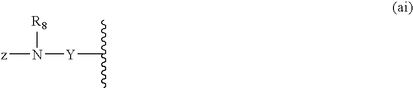

wherein Y is a (C₁-C₆) alkylene group,

Z is hydrogen or a (C₁-C₆)alkyl group,

R₈ is hydrogen, or an optionally substituted group selected from (C₁-C₆)alkyl, (C₁-C₆)alkyl sulphonyl or (C₂-C₆)alkanoyl, wherein optional substituents are one or more groups selected from halo, trifluoromethyl, cyano, nitro, hydroxy, amino, carboxy, carbamoyl, sulfamoyl, (1-6C)alkoxy, (1-6C)alkylthio, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or Q₁ is

(ii) a group of sub-formula (bi):

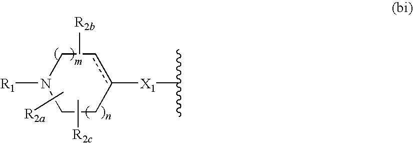

wherein:
the “- - - -” is either a bond or is absent;
X₁ is a bond or (C₁-C₄)alkylene;
R₁ is
   (a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl;
   (b)

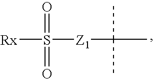

wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl;
(c)

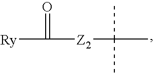

wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)Cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;

R₄ is selected from hydrogen, (1-6C)alkyl, aryl, aryl-(1-6C)alkyl, heterocyclyl, heteroaryl, heteroaryl-(1-6C)alkyl and heterocyclyl-(1-6C)alkyl, any of which optionally bears on carbon one or more R²¹ substituents, which may be the same or different,

R₅ is aryl which is ortho-substituted with at least one group selected from (C₁-C₃)alkyl, halogen and halo-(1-3C)alkyl, and which is optionally additionally substituted with 1 or 2 groups selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

R₁₃—X₂—

wherein X₂ is a direct bond or is selected from O, S, SO, SO₂, N(R₁₄), CO, CH(OR₁₄), CON(R₁₄), N(R₁₄)CO, SO₂N(R₁₄), N(R₁₄)SO₂, OC(R₁₄)₂, SC(R₁₄)₂ and N(R₁₄)C(R₁₄)₂, wherein R₁₄ is hydrogen or (1-6C)alkyl, and R₁₃ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl; and

R²¹ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

—X⁴—R¹⁶

wherein X⁴ is a direct bond or is selected from O, CO and N(R¹⁶), wherein R¹⁶ is hydrogen or (1-6C)alkyl, and R¹⁶ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:

—X³-Q²

wherein X³ is a direct bond or is selected from O, S, SO, SO₂, N(R¹⁷), CO, CH(OR¹⁷), CON(R¹⁷), N(R¹⁷)CO, SO₂N(R¹⁷), N(R¹⁷)SO₂, OC(R¹⁷)₂, SC(R¹⁷)₂ and N(R¹⁷)C(R¹⁷)₂, wherein R¹⁷ is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R²¹ optionally bears on carbon one or more R¹⁸,

and wherein any if any heteroaryl or heterocyclyl group within R²¹ contains an —NH— moiety, the nitrogen of said moiety optionally bears a group selected from R¹⁹,

and wherein any heterocyclyl group within a substituent R²¹ optionally bears 1 or 2 oxo or thioxo substituents;

R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;

R¹⁹ is selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:

—X⁶-Q⁴

wherein X⁶ is a direct bond or is selected from CO, SO₂, CON(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl.

Suitable optional substitutents for any of the R₁ groups or the moieties such as Z₁, Rₓ, Z₂ and R_{y} within it are independently selected from (C₁-C₃)alkyl, (C₁-C₃)alkoxy, halo, cyano, —OH, —CF₃, —OCF₃, —NR₁₆R₁₇ (for example, —NH₂, —NH(C₁-C₆)alkyl or —N[(C₁-C₆)alkyl)]₂), —NHCOR₁₆, —N[(C₁-C₆)alkyl]C(O)R₁₆, —C(O)NR₁₆R₁₇, —C(O)(C₁-C₄)alkyl), —SO₂(C₁-C₄)alkyl) and —SO₂NR₁₆R₁₇; wherein R₁₆ and R₁₇ are independently selected from hydrogen and (C₁-C₄)alkyl, or R₁₆ and R₁₇ together with the nitrogen to which they are attached form a 4- to 6-membered heterocyclyl group, for example an azetidinyl, pyrrolidinyl, piperidinyl, piperazinyl or morpholinyl ring;

or two adjacent substituents on an aryl group within an R₁ group form a (C₁-C₄)alkylenedioxy group such as methylenedioxy.

In a particular, in the compounds of formula (I), Q₁ is a group of sub-formula (bi).

A particular group of compounds of formula (I) are compounds of formula (I′)

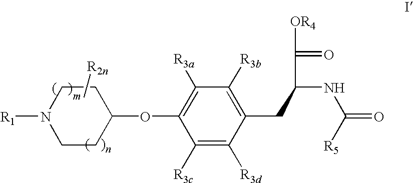

or a pharmaceutical acceptable salt thereof, wherein:
R₁ is
   (a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl; or R¹ is
   (b)

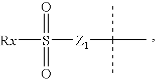

wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl; or R¹ is
(c)

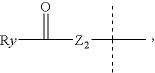

wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₙ is 0, 1, or 2 groups, each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ is aryl which is ortho-substituted with at least one group selected from (C₁-C₃)alkyl or halogen, and which is optionally additionally substituted with 1 or 2 groups selected from (C₁-C₃)alkyl, (C₁-C₃)alkoxy, or halogen.

What is also provided is a compound of formula IA

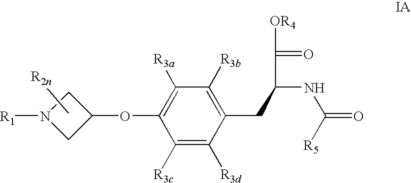

or a pharmaceutically acceptable salt thereof, wherein R₁, R₂ₙ, R_{3a-d}, R₄ and R₅ are as defined for a compound of formula I.

What is also provided is a compound of formula IB

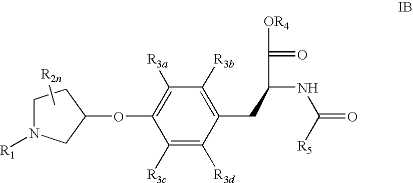

or a pharmaceutically acceptable salt thereof, wherein R₁, R_{3a-d}, R₄, and R₅ are as defined for a compound of formula I and R₂ₙ is as defined for formula (I′).

What is also provided is a compound of formula IC-1 or IC-2

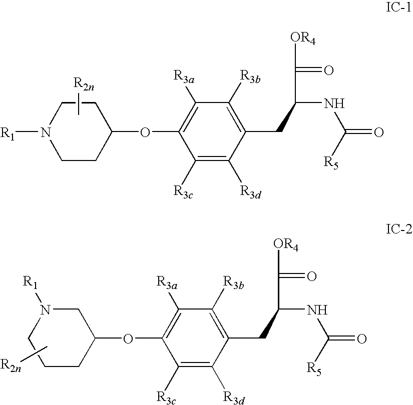

or a pharmaceutically acceptable salt thereof, wherein R₁, R_{3a-d}, R₄, and R₅ are as defined for a compound of formula I and R₂ₙ is as defined for formula (I′).

What is also provided is a compound of formula ID-1 or ID-2

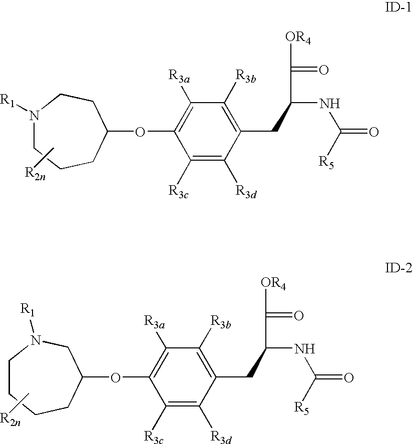

or a pharmaceutically acceptable salt thereof, wherein R₁, R_{3a-d}, R₄, and R₅ are as defined for a compound of formula I and R₂ₙ is as defined for formula (I′).

In a particular embodiment of the invention, R₅ is a group of sub-formula (iii)

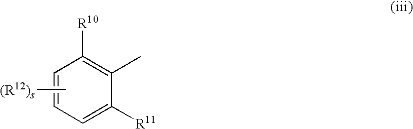

wherein:

R¹⁰ is selected from halo such as chloro, (1-3C)alkyl and halo-(1-3C)alkyl, and in particular is chloro;

s is 0, 1, 2 or 3

each R¹², which may be the same or different, is selected from

halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:

R₁₃—X₂—

wherein R₁₃ and X₂ are as defined in relation to formula (I), and
R₁₁ is selected from hydrogen or a group as listed above for R₁₂.

In particular R₁₀ is halo such as chloro.

In a particular embodiment, R₁₁ is selected from hydrogen, (C₁₋₃)alkyl, (C₁₋₃)alkoxy, halogen, cyano or heterocycyl such as pyrrolidinyl, and each R₁₂ is independently selected from C₁₋₃)alkyl, (C₁₋₃)alkoxy, halogen, cyano or heterocycyl such as pyrrolidinyl,

In another particular embodiment, s is 0 or 1, and is preferably 0.

For instance, R₁₁ is selected from hydrogen, (C₁₋₃)alkyl, (C₁₋₃)alkoxy or halogen, such as chloro.

In other examples, each R₁₂ is independently selected from (C₁₋₃)alkyl, (C₁₋₃)alkoxy or halogen

Suitably s is 0. Particular examples of compounds of formula (iii) which may constitute an R₅ group are listed below.

In another embodiment, Q₁ is a group of sub-formula (ai). In particular, in this case, Y is a C₁₋₃alkylene group such as methylene or ethylene or propylene.

Examples of Z groups include hydrogen and C₁₋₃ alkyl groups such as methyl, ethyl or propyl.

Examples of groups R₈ are hydrogen, or an optionally substituted group selected from (C₁-C₆)alkyl, (C₁-C₆)alkyl sulphonyl wherein optional substituents are as defined above. In particular R₈ is hydrogen, or unsubstituted (C₁-C₆)alkyl (such as methyl or ethyl) or unsubstituted (C₁-C₆)alkyl sulphonyl such as methylsulphonyl or ethylsulphonyl.

In another embodiment, R₈ is a (C₂-C₆)alkanoyl such as acetyl.

In another embodiment, Q₁ is a group of sub-formula (II). In such cases, examples of X₁ groups include bonds or C₁₋₃alkylene groups, such as methylene.

When Q₁ is a group of sub-formula (II), the “- - - -” is absent in a particular embodiment, so that the nitrogen containing ring is a saturated ring.

In particular embodiments the sum of m+n is equal to 0, 1, 2 or 3, so that the nitrogen containing ring contains from 4 to 7 atoms.

In a particular embodiment, R₁ is selected from hydrogen, optionally substituted alkyl (such as methyl), optionally substituted aryl such as optionally substituted phenyl, optionally substituted aralkyl, such as optionally substituted benzyl, optionally substituted heterocyclylalkyl (such as quinolinylmethyl), optionally substituted heteroaryl (such as indolyl,) wherein any optional substituents are selected from the groups defined above, and in particular are one or more groups selected from (C₁-C₃)alkyl such as methyl, (C₁-C₃)alkoxy such as methoxy, halo (such as fluoro, chloro), cyano, —CF₃, —OCF₃, —C(O)NR₁₆R₁₇, and —SO₂(C₁-C₄)alkyl) such as methylsulphonyl; wherein R₁₆ and R₁₇ are independently selected from hydrogen and (C₁-C₄)alkyl such as methyl.

In another embodiment, R₁ is selected from a group (b)

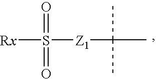

wherein [symbol] indicates the point of attachment and Z₁ is absent and Rₓ is an optionally substituted group selected from (C₁-C₆)alkyl wherein the substituents are as defined above. A particular example of a group Rₓ is methyl.

In another embodiment, R₁ is a group (c) where

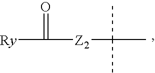

wherein [symbol] indicates the point of attachment and Z₂ is absent and R_{y} is an optionally substituted group selected from (C₁-C₆)alkyl, optionally substituted aryl such as optionally substituted phenyl, optionally substituted heteroaryl such as thienyl, benzisoxazolyl. Suitable optional substituents are as defined above, but in particular may be selected from (C₁-C₃)alkyl such as methyl, hydroxy, methoxy, halo (such as fluoro, chloro), or NR₁₆R₁₇, wherein R₁₆ and R₁₇ are independently selected from hydrogen and (C₁-C₄)alkyl such as methyl.

Particular novel compounds of the invention include, for example, compounds of the formula I, or pharmaceutically acceptable salts and pro-drugs thereof, wherein, unless otherwise stated, each of Q₁, R₃ₐ, R_{3b}, R_{3c}, R_{3d}, R₄ or R₅ has any of the meanings defined hereinbefore or in paragraphs (1) to (46) hereinafter:—

(1) R₅ is a group of sub-formula (iii) above in which R₁₀ is selected from chloro, (1-3C)alkyl and trifluoromethyl;
(1a) R₅ is an aryl group which is substituted at the ortho position by a (C₁-C₃)alkyl or halogen, and which is optionally additionally substituted with 1 or 2 groups selected from (C₁-C₃)alkyl, (C₁-C₃)alkoxy, or halogen;
(2) R₅ is a group of sub-formula (iii) above in which R₁₀ is selected from chloro and (1-3C)alkyl;
(3) R₅ is a group of sub-formula (iii) above in which R₁₀ is selected from chloro, methyl and ethyl;
(4) R₅ is a group of sub-formula (iii) above in which R₁₀ is methyl or ethyl;
(5) R₅ is a group of sub-formula (iii) above in which R₁₀ is methyl or trifluoromethyl;
(6) R₅ is a group of sub-formula (iii) above in which R₁₀ is chloro or methyl;
(7) R₅ is a group of sub-formula (iii) above in which R₁₀ is chloro;
(8) R₅ is a group of sub-formula (iii) above in which R₁₀ is methyl;
(9) R₅ is a group of sub-formula (iii) above in which R₁₁ is selected from hydrogen, halo, trifluoromethyl, (1-6C)alkyl, and (1-6C)alkoxy;
(10) R₅ is a group of sub-formula (iii) above in which R₁₁ is selected from hydrogen, halo and (1-4C)alkyl,
(11) R₅ is a group of sub-formula (iii) above in which R₁₁ is selected from hydrogen, fluoro, chloro, bromo and methyl;
(12) R₅ is a group of sub-formula (iii) above in which R₁₁ is selected from hydrogen and chloro;
(13) R₅ is a group of sub-formula (iii) above in which R₁₁ is hydrogen;
(14) R₅ is a group of sub-formula (iii) above in which R₁₁ is fluoro or chloro;
(15) R₅ is a group of sub-formula (iii) above in which R₁₁ is chloro;
(16) R₅ is a group of sub-formula (iii) above in which R₁₀ is chloro or methyl and R₁₁ is selected from hydrogen, fluoro, chloro and methyl;
(17) R₅ is a group of sub-formula (iii) above in which R₁₀ and R₁₁ are both chloro;
(18) R₅ is a group of sub-formula (iii) above in which m is 0, 1 or 2 and each R₁₂, which may be the same or different, is selected from

halo, trifluoromethyl, cyano, nitro, hydroxy, mercapto, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

(19) R₅ is a group of sub-formula (iii) above in which m is 0, 1 or 2 and each R₁₂, which may be the same or different, is selected from halo and (1-4C)alkyl,
(20) R₅ is a group of sub-formula (iii) above in which m is 0, 1 or 2 and each R₁₂, which may be the same or different, is selected from fluoro, chloro, bromo and (1-3C)alkyl;
(21) R₅ is a group of sub-formula (iii) above in which m is 0 or 1 and R₁₂ is selected from fluoro, chloro and methyl;
(22) R₅ is a group of sub-formula (iii) above in which m is 0;
(23) R₄ is selected from, hydrogen, (1-6C)alkyl, phenyl, monocyclic heteroaryl (as defined herein), benzyl or (1-3C)alkyl(monocyclic heteroaryl),
(23a) R₄ is selected from monocyclic heterocyclyl or heterocyclyl(1-6C)alkyl,
(23b) R₄ is selected from hydrogen, methyl, ethyl, hydroxyethyl, iso-propyl, 2-(diethylaminoethyl):

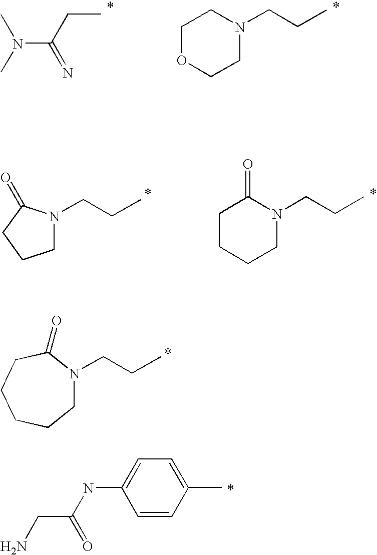

where * indicates the point of attachment to the oxygen atom,
(24) R⁴ is selected from hydrogen and (1-6C)alkyl,
(25) R⁴ is selected from hydrogen and (1-4C)alkyl,
(26) R⁴ is hydrogen;
(27) R₃ₐ, R_{3b}, R_{3c} and R_{3d} are independently selected from H, halo, (C₁₋₃)alkyl or (C₁₋₃)alkoxy, provided that at least to of said groups are hydrogen,
(28) R₃ₐ, R_{3b}, R_{3c} and R_{3d} are independently selected from H, halo, methyl or methoxy,

### (29) R₃ₐ, R_{3b}, R_{3c} and R_{3d} are all H,

(30) Q₁ is a group of sub-formula (bi) where X₁ is a bond or methylene,
(31) Q₁ is a group of sub-formula (bi) where “- - - - -” is a bond.
(32) Q₁ is a group of sub-formula (bi) in which “- - - -” is absent.
(33) R₁ is selected from optionally substituted (C₁-C₆)alkyl, aralkyl (for example optionally substituted benzyl or phenylethyl) or heteroaralkyl; or R₁ is

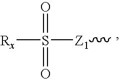

wherein [symbol] indicates the point of attachment, Z₁ is absent and Rₓ is an optionally substituted group selected from (C₁-C₆)alkyl, (C₃-C₆)cycloalkyl, (C₃-C₆)cycloalkyl(C₁-C₃)alkylene, aryl, heteroaryl, benzyl and heteroaryl(C₁-C₃)alkyl; or R₁ is

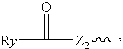

wherein [symbol] indicates the point of attachment, Z₂ is absent, R_{y} is an optionally substituted group selected from (C₁-C₆)alkyl, (C₁-C₆)alkoxy, (C₃-C₆)cycloalkyl, (C₃-C₆)cycloalkyl(C₁-C₃)alkylene, aryl, heterocyclcloalkyl, heteroaryl, benzyl, heteroaryl(C₁-C₃)alkyl, heterocyclcloalkyl(C₁-C₃)alkylene and NR′R″, wherein R′ and R″ are each independently H or optionally substituted (C₁-C₆)alkyl, phenyl or benzyl or R′ and R″ taken together with the nitrogen to which they are attached form an optionally substituted 4, 5 or 6-membered ring;

and wherein the optional substituents that may be present on R₁ are independently selected from (C₁-C₃)alkyl, (C₁-C₃)alkoxy, halo, cyano, —OH, —CF₃, —OCF₃, —NR₁₆R₁₇ (for example, —NH₂, —NH(C₁-C₆)alkyl or —N[(C₁-C₆)alkyl)]₂), —NHCOR₁₆, —N[(C₁-C₆)alkyl]C(O)R₁₆, —C(O)NR₁₆R₁₇, —C(O)(C₁-C₄)alkyl), —SO₂(C₁-C₄)alkyl) and —SO₂NR₁₆R₁₇; wherein R₁₆ and R₁₇ are independently selected from hydrogen and (C₁-C₄)alkyl, or R₁₆ and R₁₇ together with the nitrogen to which they are attached form a 4- to 6-membered heterocyclyl group, for example an azetidinyl, pyrrolidinyl, piperidinyl, piperazinyl or morpholinyl ring;

or two adjacent substituents on an aryl group within an R₁ group form a (C₁-C₄)alkylenedioxy group such as methylenedioxy.

(34) R₁ is hydrogen or optionally substituted (C₁-C₄)alkyl;
(35) R₁ is optionally substituted aralkyl selected from optionally substituted benzyl or phenylethyl;
(36) R₁ is optionally substituted heteroaryl(C₁-C₃)alkyl (for example, optionally substituted 1H-pyrazolyl(C₁-C₃)alkyl, 1,3-thiazolyl(C₁-C₃)alkyl, isoxazolyl(C₁-C₃)alkyl, 1H 1,2,4-triazolyl(C₁-C₃)alkyl, pyridinyl(C₁-C₃)alkyl, benzisoxazolyl(C₁-C₃)alkyl or benzimidazolyl(C₁-C₃)alkyl),

### (37) R₁ is

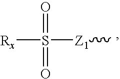

wherein [symbol] indicates the point of attachment, Z₁ is absent, and Rₓ is an optionally substituted group selected from optionally substituted (C₁-C₄)alkyl, optionally substituted (C₃-C₆)cycloalkyl (for example cyclopropyl, cyclobutyl, cyclopentyl or cyclohexyl), optionally substituted (C₃-C₆)cycloalkyl(C₁-C₃)alkylene (for example optionally substituted cyclopropylmethyl, cyclobutylmethyl, cyclopentylmethyl or cyclohexylmethyl), optionally substituted phenyl, optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl),
(38) R₁ is optionally substituted benzyl.
(39) R₁ is optionally substituted heteroaryl(C₁-C₃)alkylene (for example optionally substituted 1H-pyrazolyl(C₁-C₃)alkyl, 1,3-thiazolyl(C₁-C₃)alkyl, isoxazolyl(C₁-C₃)alkyl, 1H 1,2,4-triazolyl(C₁-C₃)alkyl, pyridinyl(C₁-C₃)alkyl, benzisoxazolyl(C₁-C₃)alkyl or benzimidazolyl(C₁-C₃)alkyl),

### (40) R₁ is

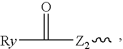

wherein [symbol] indicates the point of attachment, Z₂ is absent, and

R_{y} is an optionally substituted group selected from (i) to (x):
(i) optionally substituted (C₁-C₄)alkyl;
(ii) optionally substituted (C₃-C₆)cycloalkyl (for example optionally substituted cyclopropyl, cyclobutyl, cyclopentyl or cyclohexyl);
(iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
(iv) optionally substituted heterocyclyl (for example optionally substituted 1,1-dioxotetrahydrothiopyranyl, tetrahydropyranyl, 1,1-dioxotetrahydrothienyl or piperidinyl);
(v) optionally substituted phenyl;
(vi) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
(vii) optionally substituted benzyl;
(viii) optionally substituted heteroaryl(C₁-C₃)alkyl (for example optionally substituted 1H-pyrazolyl(C₁-C₃)alkyl, 1,3-thiazolyl(C₁-C₃)alkyl, isoxazolyl(C₁-C₃)alkyl, 1H 1,2,4-triazolyl(C₁-C₃)alkyl, pyridinyl(C₁-C₃)alkyl, benzisoxazolyl(C₁-C₃)alkyl or benzimidazolyl(C₁-C₃)alkyl);
(ix) optionally substituted heterocyclyl(C₁-C₃)alkyl (for example optionally substituted 1,1-dioxotetrahydrothiopyranylmethyl, 1,1-dioxotetrahydrothienylmethyl or tetrahydropyranylmethyl); and
(x) NR′R″, wherein R′ and R″ are each independently H or optionally substituted (C₁-C₄)alkyl, phenyl or benzyl or R′ and R″ taken together with the nitrogen to which they are attached form an optionally substituted azetidinyl, pyrrolidinyl, piperidinyl or morpholinyl ring;
   (41) where R₁ is or comprises an alkyl, cycloalkyl, cycloalkyl-alkylene, heterocyclcloalkyl or heterocyclcloalkyl(C₁-C₃)alkyl group, the group is optionally substituted by one or more (for example 1, 2 or 3) substituents selected from hydroxy, cyano, —CF₃, (C₁-C₃)alkoxy, —NR₁₆R₁₇ (for example, —NH₂, —NH(C₁-C₃)alkyl or —N[(C₁-C₃)alkyl)₂), —C(O)NR₁₆R₁₇, —NHC(O)R₁₆ or —N[(C₁-C₆)alkyl]C(O)R₁₆; where wherein R₁₆ and R₁₇ are independently hydrogen or (C₁-C₄)alkyl, or R₁₆ and R₁₇ together with the nitrogen to which they are attached form a 4- to 6-membered ring (for example a monocyclic nitrogen containing heterocyclyl group, such as azetidinyl, pyrrolidinyl, piperidinyl, piperazinyl or morpholinyl),
   (42) wherein R₁ is or comprises phenyl, benzyl, heteroaryl or heteroaryl(C₁-C₃)alkyl group, the group is optionally substituted by 1 or more (for example 1, 2 or 3) substituents selected from (C₁-C₃)alkyl, (C₁-C₃)haloalkyl (such as CF₃), (C₁-C₃)alkoxy, (C₁-C₃)alkylthio, halo, nitro, cyano, hydroxy, —C(O)OR₁₆ (for example —C(O)OH and —C(O)O(C₁-C₆)alkyl), —NR₁₆R₁₇ (for example, —NH₂, —NH(C₁-C₆)alkyl or —N[(C₁-C₆)alkyl)₂), —C(O)NR⁶R⁷, —NHC(O)R⁶, —N[(C₁-C₆)alkyl]C(O)R⁶, —SO₂R₁₆, hydroxy-(C₁-C₃)alkyl-, (C₁-C₃)alkoxy-(C₁-C₃)alkyl- and NR₁₆R₁₇—(C₁-C₃)alkyl-, or; wherein R₁₆ and R₁₇ are independently hydrogen or (C₁-C₄)alkyl, or R₁₆ and R₁₇ together with the nitrogen to which they are attached form a 4- to 6-membered ring (for example a monocyclic nitrogen containing heterocyclyl group, such as azetidinyl, pyrrolidinyl, piperidinyl, piperazinyl or morpholinyl); or two adjacent substituents on a phenyl ring in R₁ form a methylenedioxy or ethylenedioxy group.
   (43) R₁ is selected from optionally substituted aralkyl (for example benzyl); or R₁ is

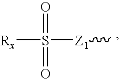

wherein [symbol] indicates the point of attachment, Z₁ is absent and Rₓ is an optionally substituted group selected from (C₁-C₆)alkyl, (C₃-C₆)cycloalkyl, (C₃-C₆)cycloalkyl(C₁-C₆)alkylene, aryl, heteroaryl benzyl and heteroarylmethyl; or R₁ is

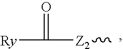

wherein [symbol] indicates the point of attachment, Z₂ is absent, R_{y} is an optionally substituted group selected from (C₁-C₆)alkyl, (C₁-C₆)alkoxy, (C₃-C₆)cycloalkyl, (C₃-C₆)cycloalkyl(C₁-C₆)alkylene, aryl, heteroaryl, benzyl and heteroarylmethyl;

and wherein the optional substituents that may be present on R₁ are independently selected from (C₁-C₃)alkyl, (C₁-C₃)alkoxy, phenyl, halo, cyano, —OH, —CF₃, —OCF₃, —NR₁₆R₁₇ (for example, —NH₂, —NHC₁-C₆alkyl or —N[(C₁-C₆)alkyl)]₂), —NHCOR₁₆, —N[(C₁-C₆)alkyl]C(O)R₁₆, —C(O)NR₁₆R₁₇, —C(O)(C₁-C₄)alkyl), —SO₂(C₁-C₄)alkyl) and —SO₂NR₁₆R_{′7}; wherein R₁₆ and R₁₇ are independently selected from hydrogen and (C₁-C₄)alkyl;

or two adjacent substituents on an aryl group within an R₁ group form a (C₁-C₄)alkylenedioxy group such as methylenedioxy.

(44) R₁ is an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl,
(45) R₁ is aralkyl which optionally bears 1, 2 or 3 substituents selected from (C₁-C₃)alkyl, (C₁-C₃)alkoxy, halo, cyano, —OH, —CF₃, —OCF₃, —NR₁₆R₁₇ (for example, —NH₂, —NHC₁-C₆alkyl or —N[(C₁-C₆)alkyl)]₂), —NHCOR⁶, —N[(C₁-C₆)alkyl]C(O)R₁₆, —C(O)NR⁶R⁷, —C(O)(C₁-C₄)alkyl), —SO₂(C₁-C₄)alkyl) and —SO₂NR₁₆R₁₇; wherein R₁₆ and R₁₇ are independently selected from hydrogen and (C₁-C₄)alkyl, or R₁₆ and R₁₇ together with the nitrogen to which they are attached form a 4- to 6-membered heterocyclyl group, for example an azetidinyl, pyrrolidinyl, piperidinyl, piperazinyl or morpholinyl ring;

or two adjacent substituents on an aryl group within an R₁ group form a (C₁-C₄)alkylenedioxy group such as methylenedioxy.

(46) R₁ is benzyl which is optionally substituted as hereinbefore defined, for example R₁ is benzyl optionally substituted by 1, 2 or 3 substituents selected from (C₁-C₃)alkyl, (C₁-C₃)alkoxy, halo (such as fluoro, chloro or bromo), cyano, hydroxy, —CF₃, —NHC(O)R⁶, —SO₂R₆, hydroxy-(C₁-C₃)alkyl- and (C₁-C₃)alkoxy-(C₁-C₃)alkyl-, wherein R₆ and R₇ are independently hydrogen or (C₁-C₃)alkyl; or two adjacent substituents on a phenyl ring in R₁ form a methylenedioxy or ethylenedioxy group.

What is also provided is a compound which is:
N-(2,6-Dichlorobenzoyl)-O-piperidin-4-yl-L-tyrosine;
N-(2,6-Dichlorobenzoyl)-O-[(3R)-pyrrolidin-3-yl]-L-tyrosine;
N-(2,6-Dichlorobenzoyl)-O-[(2S)-pyrrolidin-2-ylmethyl]-L-tyrosine;
N-(2,6-dichlorobenzoyl)-O-[2-(methylamino)ethyl]-L-tyrosine;
N-(2,6-Dichlorobenzoyl)-O-(1-methylpiperidin-4-yl)-L-tyrosine;
N-(2,6-Dichlorobenzoyl)-O-[(3R)-1-methylpyrrolidin-3-yl]-L-tyrosine;
N-(2,6-Dichlorobenzoyl)-O-{[(2S)-1-methylpyrrolidin-2-yl]methyl}-L-tyrosine;
N-(2,6-Dichlorobenzoyl)-O-(1-acetylpiperidin-4-yl)-L-tyrosine;
N-(2,6-dichlorobenzoyl)-O-(1-glycoloylpiperidin-4-yl)-L-tyrosine;
N-(2,6-Dichlorobenzoyl)-O-[1-(N,N-dimethylglycyl)piperidin-4-yl]-L-tyrosine;
O-[(3R)-1-Acetylpyrrolidin-3-yl]-N-(2,6-dichlorobenzoyl)-L-tyrosine;
N-(2,6-Dichlorobenzoyl)-O-[(3R)-1-glycoloylpyrrolidin-3-yl]-L-tyrosine;
O-{3-[Acetyl(methyl)amino]propyl}-N-(2,6-dichlorobenzoyl)-L-tyrosine;
O-{[(2R)-1-Acetylpyrrolidin-2-yl]methyl}-N-(2,6-dichlorobenzoyl)-L-tyrosine;
N-(2,6-Dichlorobenzoyl)-O-{[(2S)-1-(N,N-dimethylglycyl)pyrrolidin-2-yl]methyl}-L-tyrosine;
N-(2,6-Dichlorobenzoyl)-O-{[(2S)-1-glycoloylpyrrolidin-2-yl]methyl}-L-tyrosine;
N-(2,6-dichlorobenzoyl)-O-[1-(methylsulfonyl)piperidin-4-yl]-L-tyrosine;
N-(2,6-Dichlorobenzoyl)-O-[(3R)-1-(methylsulfonyl)pyrrolidin-3-yl]-L-tyrosine;
N-(2,6-Dichlorobenzoyl)-O-{3-[methyl(methylsulfonyl)amino]propyl}-L-tyrosine;
N-(2,6-Dichlorobenzoyl)-O-{[(2S)-1-(methylsulfonyl)pyrrolidin-2-yl]methyl}-L-tyrosine;
N-(2,6-Dichlorobenzoyl)-O-{[(2R)-1-(methylsulfonyl)pyrrolidin-2-yl]methyl}-L-tyrosine;
N-(2,6-Dichlorobenzoyl)-O-{3-[methyl(methylsulfonyl)amino]ethyl}-L-tyrosine;
N-(2,6-Dichlorobenzoyl)-O-(1-phenylpiperidin-4-yl)-L-tyrosine;
N-(2,6-Dimethylbenzoyl)-O-(1-phenylpiperidin-4-yl)-L-tyrosine;
N-(2-Chloro-6-methylbenzoyl)-O-(1-phenylpiperidin-4-yl)-L-tyrosine;
N-(2-Chloro-4,5-dimethoxybenzoyl)-O-(1-phenylpiperidin-4-yl)-L-tyrosine;
N-(2,6-dichlorobenzoyl)-O-[1-(4-fluoro-3-methylbenzoyl)piperidin-4-yl]-L-tyrosine;
N-(2,6-dichlorobenzoyl)-O-[1-(4-fluoro-3-methylbenzoyl)piperidin-4-yl]-L-tyrosine;
O-[1-(2,1-benzisoxazol-3-ylcarbonyl)piperidin-4-yl]-N-(2,6-dichlorobenzoyl)-L-tyrosine;
N-(2,6-dichlorobenzoyl)-O-[1-(4-fluoro-3-methylbenzoyl)azetidin-3-yl]-L-tyrosine;
N-(2,6-dichlorobenzoyl)-O-[1-(quinolin-4-ylcarbonyl)azetidin-3-yl]-L-tyrosine;
N-(2,6-dichlorobenzoyl)-O-[(3S)-1-(4-fluoro-3-methylbenzoyl)pyrrolidin-3-yl]-L-tyrosine;
O-[(3S)-1-(2,1-benzisoxazol-3-ylcarbonyl)pyrrolidin-3-yl]-N-(2,6-dichlorobenzoyl)-L-tyrosine;
N-(2,6-dichlorobenzoyl)-O-{1-[(2,5-dimethyl-3-thienyl)carbonyl]piperidin-4-yl}-L-tyrosine;
N-(2,6-dichlorobenzoyl)-O-{(3R)-1-[(2,5-dimethyl-3-thienyl)carbonyl]pyrrolidin-3-yl}-L-tyrosine;
N-(2,6-dichlorobenzoyl)-O-[1-(4-fluorobenzyl)azetidine-3-yl]-L-tyrosine;
N-(2,6-dichlorobenzoyl)-O-[1-(4-fluorobenzyl)azetidin-3-yl]-L-tyrosine;
N-(2,6-dichlorobenzoyl)-O-[1-(1H-indol-5-ylmethyl)azetidin-3-yl]-L-tyrosine;
N-(2,6-dichlorobenzoyl)-O-[1-(4-(trifluoromethyl)benzyl)azetidin-3-yl]-L-tyrosine;
O-{1-[3-(aminocarbonyl)benzyl]azetidin-3-yl}-N-(2,6-dichlorobenzoyl)-L-tyrosine;
O-[1-(3-chloro-4-fluorobenzyl)azetidin-3-yl]-N-(2,6-dichlorobenzoyl)-L-tyrosine;
O-[1-(4-cyanobenzyl)azetidin-3-yl]-N-(2,6-dichlorobenzoyl)-L-tyrosine;
N-(2,6-dichlorobenzoyl)-O-{1-[4-(trifluoromethoxy)benzyl]azetidin-3-yl}-L-tyrosine;
O-[(3S)-1-benzylpyrrolidin-3-yl]-N-(2,6-dichlorobenzoyl)-L-tyrosine;
O-[(3S)-1-benzylpyrrolidin-3-yl]-N-(2,6-dichlorobenzoyl)-L-tyrosine;
N-(2,6-dichlorobenzoyl)-O-((3R)-1-{4-[(dimethylamino)carbonyl]benzyl}pyrrolidin-3-yl)-L-tyrosine;
N-(2,6-dichlorobenzoyl)-O-[(3S)-1-(4-fluorobenzyl)pyrrolidin-3-yl]-L-tyrosine;
N-(2,6-dichlorobenzoyl)-O-[(3S)-1-(4-methoxybenzyl)pyrrolidin-3-yl]-L-tyrosine;
N-(2,6-dichlorobenzoyl)-O-[(3S)-1-(4-methylbenzyl)pyrrolidin-3-yl]-L-tyrosine;
O-[(3R)-1-(4-cyanobenzyl)pyrrolidin-3-yl]-N-(2,6-dichlorobenzoyl)-L-tyrosine;
N-(2,6-dichlorobenzoyl)-O-{(3S)-1-[4-(methylsulfonyl)benzyl]pyrrolidin-3-yl}-L-tyrosine;
O-[(3R)-1-(3-chloro-4-fluorobenzyl)pyrrolidin-3-yl]-N-(2,6-dichlorobenzoyl)-L-tyrosine;
N-(2,6-dichlorobenzoyl)-O-[(3R)-1-(4-methylbenzyl)piperidin-3-yl]-L-tyrosine;
N-(2,6-dichlorobenzoyl)-O-[(3R)-1-(4-methylbenzyl)piperidin-3-yl]-L-tyrosine;
O-[(3S)-1-(4-chlorobenzyl)piperidin-3-yl]-N-(2,6-dichlorobenzoyl)-L-tyrosine;
O-[(3S)-1-benzylpiperidin-3-yl]-N-(2,6-dichlorobenzoyl)-L-tyrosine;
O-[(3S)-1-(4-cyanobenzyl)piperidin-3-yl]-N-(2,6-dichlorobenzoyl)-L-tyrosine;
N-(2,6-dichlorobenzoyl)-O-((3R)-1-{4-[(dimethylamino)carbonyl]benzyl}piperidin-3-yl)-L-tyrosine;
N-(2,6-dichlorobenzoyl)-O-[(3S)-1-(4-methoxybenzyl)piperidin-3-yl]-L-tyrosine;
N-(2,6-dichlorobenzoyl)-O-[(3S)-1-(2,5-difluorobenzyl)piperidin-3-yl]-L-tyrosine;
N-(2,6-dichlorobenzoyl)-O-{(3S)-1-[4-(methylsulfonyl)benzyl]piperidin-3-yl}-L-tyrosine;
N-(2,6-dichlorobenzoyl)-O-[1-(4-fluorobenzyl)piperidin-4-yl]-L-tyrosine;
N-(2,6-dichlorobenzoyl)-O-[1-(4-fluorobenzyl)piperidin-4-yl]-L-tyrosine;
N-(2,6-dichlorobenzoyl)-O-[1-(2-methoxybenzyl)piperidin-4-yl]-L-tyrosine;
O-[1-(3-chlorobenzyl)piperidin-4-yl]-N-(2,6-dichlorobenzoyl)-L-tyrosine;
N-(2,6-dichlorobenzoyl)-O-{1-[4-(trifluoromethoxy)benzyl]piperidin-4-yl}-L-tyrosine;
N-(2,6-dichlorobenzoyl)-O-[1-(2,4-difluorobenzyl)piperidin-4-yl]-L-tyrosine;
O-[1-(3-cyanobenzyl)piperidin-4-yl]-N-(2,6-dichlorobenzoyl)-L-tyrosine;
N-(2,6-dichlorobenzoyl)-O-[1-(quinolin-8-ylmethyl)piperidin-4-yl]-L-tyrosine;
N-(2,6-dichlorobenzoyl)-O-{1-[4-(methylsulfonyl)benzyl]piperidin-4-yl}-L-tyrosine; or
O-[1-(4-chloro-3-methoxybenzyl)piperidin-4-yl]-N-(2,6-dichlorobenzoyl)-L-tyrosine,

or a pharmaceutically acceptable salt thereof.

Compounds of formula I, IA, IB, IC-1, IC-2, ID-1, or ID-2 may be in the form of prodrugs or solvates such as hydrates if this is convenient.

What is also provided is a compound of formula I, IA, IB, IC-1, IC-2, ID-1, or ID-2 or a pharmaceutically acceptable salt, prodrug, or solvate thereof in association with a pharmaceutically acceptable carrier, diluent, or excipient.

What is also provided is a compound of formula I, IA, IB, IC-1, IC-2, ID-1, or ID-2 or a pharmaceutically acceptable salt, prodrug, or solvate thereof, which is an integrin inhibitor useful for controlling pathologically angiogenic diseases, thrombosis, cardiac infarction, coronary heart diseases, arteriosclerosis, tumors, osteoporosis, inflammations or infections.

What is also provided is a method of treating a disease or condition mediated by a5b1 which comprises administering to a patient in need of such treatment a compound of formula compound of formula I, IA, IB, IC-1, IC-2, ID-1, or ID-2 or a pharmaceutically acceptable salt, prodrug, or solvate thereof.

What is also provided is a process for the preparation of a compound of formula I as summarized in Scheme 1 infra.

### DETAILED DESCRIPTION OF THE INVENTION

Unless otherwise stated, the following terms used in the specification and claims have the following meanings.

### DEFINITIONS

“Halo” means fluoro, chloro, bromo or iodo.

“(C 1-C₆)Alkyl” means a linear saturated monovalent hydrocarbon radical of one to Six carbon atoms or a branched saturated monovalent hydrocarbon radical of three to six carbon atoms, e.g., methyl, ethyl, propyl, 2-propyl, tert-butyl, sec-butyl, n-pentyl, n-hexyl, and the like. Examples of optional substituents that may be present on a (C₁-C₆)alkyl group include one or more substituents selected from (C₁-C₃)alkyl, aryl (for example phenyl), heteroaryl (for example a monocyclic heteroaryl group as defined hereinafter), (C₁-C₃)haloalkyl, (C₁-C₃)alkoxy, (C₁-C₃)alkylthio, —O(CH₂)₁₋₅CF₃, halo, nitro, cyano, ═O, ═S, —OH, —SH, —CF₃, —OCF₃, —C(O)OR₆ (for example —C(O)OH and —C(O)O(C₁-C₆)alkyl), —OC(O)R₆, —NR₆R₇ (for example, —NH₂, —NH(C₁-C₆)alkyl or —N[(C₁-C₆)alkyl)₂], —C(O)NR⁶R⁷, —NHC(O)R⁶, —N[(C₁-C₆)alkyl]C(O)R₆, —C(O)R₆, —SR₆, —SOR₆, —SO₂R₆, —SO₂NR₆R₇, hydroxy-(C₁-C₃)alkyl, (C₁-C₃)alkoxy-(C₁-C₃)alkyl and NR₆R₇—(C₁-C₃)alkyl-; wherein R₆ and R₇ are independently hydrogen, alkyl (for example (C₁-C₆)alkyl, particularly (C₁-C₄)alkyl), heteroaryl (for example a monocyclic heteroaryl group as defined hereinafter) or aryl (for example phenyl) or R₆ and R₇ together with the nitrogen to which they are attached form a 4- to 7-membered ring (for example a 4- to 7-membered nitrogen containing heterocyclyl group as defined herein, such as a monocyclic nitrogen containing heterocyclyl group, for example azetidinyl, pyrrolidinyl, piperidinyl, piperazinyl or morpholinyl). Particularly, R⁶ and R⁷ are independently selected from hydrogen, (C₁-C₄)alkyl, phenyl or R⁶ and R⁷ together with the nitrogen to which they are attached form a 4- to 7-membered heterocyclyl group, for example pyrrolidinyl, piperidinyl, piperazinyl or morpholinyl.

An “alkylene,” “alkenylene,” or “alkynylene” group is an alkyl, alkenyl, or alkynyl group that is positioned between and serves to connect two other chemical groups. Thus, “(C₁-C₆)alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like. (C₁-C₆)alkylene may be substituted with one or more of the substituents selected from those provided for (C₁-C₆)alkyl.

“(C₂-C₆)Alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like. (C₁-C₆)Alkenylene may be substituted with one or more of the substituents selected from those provided for (C₁-C₆)alkyl.

“(C₂-C₆)Alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like. (C₁-C₆)alkynylene may be substituted with one or more of the substituents selected from those provided for (C₁-C₆)alkyl.

“(C₃-C₆)Cycloalkyl” means a hydrocarbon ring containing from 3 to 6 carbon atoms, for example, cyclopropyl, cyclobutyl, cyclopentyl, cyclohexyl. Where possible, the cycloalkyl group may contain double bonds, for example, 3-cyclohexen-1-yl. The cycloalkyl ring may be optionally substituted as provided for (C₁-C₆)alkyl, or two adjacent substituents on a (C₃-C₆)cycloalkyl group together with the carbon atoms to which they are attached form a phenyl ring which is fused to the (C₃-C₆)cycloalkyl group, for example two adjacent substituents on a cyclopropyl ring together with the carbon atoms to which they are attached form a phenyl ring to give a 2,3-dihydro-1H-inden-2yl group. For example, a (C₃-C₆)cycloalkyl group may be unsubstituted or substituted by 1 to 3 substituents selected from (C₁-C₃)alkyl, (C₁-C₃)haloalkyl, (C₁-C₃)alkoxy, hydroxy, thiol, nitro, halogen, amino, (C₁-C₃)alkylamino and di-[(C₁-C₃)]alkyl]amino, formyl, carboxyl, —CN, —NHCOR⁶, —CONHR⁶, —CO₂R⁶, —COR⁶, aryl, or heteroaryl, wherein R⁶, alkyl, aryl, and heteroaryl are as defined herein. Examples of substituted cycloalkyl groups include 1-cyanocyclopropyl, fluorocyclopropyl, 2-iodocyclobutyl, 2,3-dimethylcyclopentyl, 2,2-dimethoxycyclohexyl or 3-phenylcyclopentyl.

“(C₃-C₆)Cycloalkyl(C₁-C₆)alkylene” means a (C₃-C₆)cycloalkyl group covalently attached to a (C₁-C₆)alkylene group, both of which are defined herein, for example cyclopropylmethyl, cyclobutylmethyl, cyclopentylmethyl or cyclohexylmethyl. (C₃-C₆)Cycloalkyl(C₁-C₆)alkylene may be optionally substituted as provided for (C₁-C₆)alkyl.

“(C₁-C₆)alkoxy” includes for example methoxy, ethoxy, propoxy and isopropoxy. (C₁-C₆)alkoxy may be optionally substituted as provided for (C₁-C₆)alkyl.

The term “heterocyclyl” or “heterocyclic” means non-aromatic, monocyclic, fused, bridged, or spiro bicyclic saturated or partially saturated heterocyclic ring system(s) which optionally may be substituted with up to 4 groups selected from those recited above as substituents for alkyl. Monocyclic heterocyclic rings contain from about 3 to 12 ring atoms, with from 1 to 5 heteroatoms selected from N, O, and S, and preferably from 3 to 7 member atoms, in the ring. Bicyclic heterocycles contain from 7 to 17 member atoms, preferably 7 to 12 member atoms, in the ring. Bicyclic heterocycles contain from about 7 to about 17 ring atoms, preferably from 7 to 12 ring atoms. Bicyclic heterocyclic(s) rings may be fused, spiro, or bridged ring systems. Partially saturated heterocycles are heterocyclic ring systems that are not completely saturated and include partially aromatic ring systems in the sense that one ring of a fused ring system may be aromatic and the other non-aromatic, for example indoline. Examples of heterocyclic groups include cyclic ethers (oxiranes) such as ethyleneoxide, tetrahydrofuran, tetrahydropyran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiepin-4-yl. Other commonly employed heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene and tetrahydrothiopyran.

“Heterocyclyl(C₁-C₆)alkylene” means a heterocyclyl group covalently attached to a (C₁-C₆)alkylene group, both of which are defined herein, for example pyrrolidinylmethyl, piperidinylmethyl, morpholinylmethyl and piperazinylmethyl. (C₃-C₆)Heterocyclyl(C₁-C₆)alkylene may be optionally substituted as provided for (C₁-C₆)alkyl.

The term “aryl” means a cyclic or polycyclic aromatic ring having from 5 to 12 carbon atoms. Aryl may be unsubstituted or substituted with up to 4 groups selected from those recited above as substituents for (C₁-C₆)alkyl; or two substituents on the aryl ring form a (C₁-C₄)alkylenedioxy group (for example two adjacent substituents form a methylenedioxy or ethylenedioxy group); or two substituents on the aryl ring form a (C₃-C₆)cycloalkyl group (for example two adjacent substituents on a phenyl ring, together with the phenyl ring to which they are attached form a 2,3-dihydroindenyl group). The term aryl includes both monovalent species and divalent species. Examples of aryl groups include, but are not limited to, phenyl, biphenyl, naphthyl, each of which may be optionally substituted with 1 or more (for example 1 to 4) substituents as defined above as substituents for (C₁-C₆)alkyl, examples of substituted aryl include 2-chlorophenyl, 3-chlorophenyl, 4-chlorophenyl, 2-fluororophenyl, 3-fluorophenyl, 4-fluorophenyl, 2-methylphenyl, 3-methylphenyl, 4-methylphenyl, 2-hydroxyphenyl, 3-hydroxyphenyl, 4-hydroxyphenyl, 2-methoxyphenyl, 3-methoxyphenyl, 4-methoxyphenyl, 2-aminophenyl, 2-cyanophenyl, 3-cyanophenyl, 4-cyanophenyl, 4-methylsulfonylphenyl, 4-acetylaminophenyl, 3-pyrrolidinylphenyl, 4-hydroxymethylphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, 2-trifluoromethylphenyl, 3-trifluoromethylphenyl, 4-trifluoromethylphenyl and the like.

Aralkyl means an aryl group covalently attached to a (C₁-C₆)alkylene group, both of which are defined herein. Aralkyl may be optionally substituted as provided for (C₁-C₆)alkyl. Examples of aralykl groups include benzyl, phenylethyl, 3-(3-chlorophenyl)-2-methylpentyl, 2-chlorobenzyl, 3-chlorobenzyl, 4-chlorobenzyl, 2-fluorobenzyl, 3-fluorobenzyl, 4-fluorobenzyl, 2-methylbenzyl, 3-methylbenzyl, 4-methylbenzyl, 2-hydroxybenzyl, 3-hydroxybenzyl, 4-hydroxybenzyl, 2-methoxybenzyl, 3-methoxybenzyl, 4-methoxybenzyl, 2-aminobenzyl, 2-cyanobenzyl, 3-cyanobenzyl, 4-cyanobenzyl, 4-methylsulfonylbenzyl, 4-acetylaminobenzyl, 2-chloro-3-methylbenzyl, 2-chloro-4-methylbenzyl, 2-chloro-5-methylbenzyl, 3-chloro-2-methylbenzyl, 3-chloro-4-methylbenzyl, 4-chloro-2-methylbenzyl, 4-chloro-3-methylbenzyl, 5-chloro-2-methylbenzyl, 2,3-dichlorobenzyl, 2,5-dichlorobenzyl, 3,4-dichlorobenzyl, 2,3-dimethylbenzyl, 3,4-dimethylbenzyl, and the like.

The term “heteroaryl” means an aromatic mono-, bi- or polycyclic ring incorporating one or more (i.e. 1-4) heteroatoms selected from N, O and S. Heteroaryl may be unsubstituted or substituted with up to 4 groups selected from those recited above as substituents for (C₁-C₆)alkyl. The term heteroaryl includes both monovalent species and divalent species. Examples of monocyclic heteroaryl include, but are not limited to substituted or unsubstituted thienyl, furanyl, pyrrolyl, imidazolyl, pyrazolyl, thiazolyl, isothiazolyl, isoxazolyl, oxazolyl, thiadiazolyl, triazolyl, tetrazolyl, pyridinyl, pyrazinyl or pyrimidinyl. Monocyclic diheteroaryl groups (monocyclic heteroaromatic groups with 2 heteroatoms) include, but are not limited to, 1-, 2-, 4- or 5-imidazolyl, 1-, 3-, 4- or 5-pyrazolyl, 3-, 4- or 5-isothiazolyl, 3-, 4- or 5-isoxazolyl, 2-pyrazinyl, 2-, 4- or 5-pyrimidinyl. Examples of monocyclic heteroaromatic groups with 3 or more heteroatoms include, but are not limited to, 1-, 3- or 5-triazolyl, 1-, 2- or 3-tetrazolyl, 1,2,5-thiadazol-3yl or 1,2,3-thiadiazol-5yl). Examples of bicyclic and polycyclic heteroaryl groups include but are not limited to 1-, 2-, 3-, 5-, 6-, 7- or 8-indolizinyl, 1-, 3-, 4-, 5-, 6- or 7-isoindolyl, 2-, 3-, 4-, 5-, 6- or 7-indolyl, 2-, 3-, 4-, 5-, 6- or 7-indazolyl, 2-, 4-, 5-, 6-, 7- or 8-purinyl, 1-, 2-, 3-, 4-, 6-, 7-, 8- or 9-quinolizinyl, 2-, 3-, 4-, 5-, 6-, 7- or 8-quinolinyl, 1-, 3-, 4-, 5-, 6-, 7- or 8-isoquinolinyl, 1-, 4-, 5-, 6-, 7- or 8-phthalazinyl, 2-, 3-, 4-, 5- or 6-naphthyridinyl, 2-, 3-, 5-, 6-, 7- or 8-quinazolinyl, 3-, 4-, 5-, 6-, 7- or 8-cinnolinyl, 2-, 4-, 6- or 7-pteridinyl, 1-, 2-, 3-, 4-, 5-, 6-, 7- or 8-4aH carbazolyl, 1-, 2-, 3-, 4-, 5-, 6-, 7- or 8-carbazolyl, 1-, 3-, 4-, 5-, 6-, 7-, 8- or 9-carbolinyl, 1-, 2-, 3-, 4-, 6-, 7-, 8-, 9- or 10-phenanthridinyl, 1-, 2-, 3-, 4-, 5-, 6-, 7-, 8- or 9-acridinyl, 1-, 2-, 4-, 5-, 6-, 7-, 8- or 9-perimidinyl, 2-, 3-, 4-, 5-, 6-, 8-, 9- or 10-phenathrolinyl, 1-, 2-, 3-, 4-, 6-, 7-, 8- or 9-phenazinyl, 1-, 2-, 3-, 4-, 6-, 7-, 8-, 9- or 10-phenothiazinyl, 1-, 2-, 3-, 4-, 6-, 7-, 8-, 9- or 10-phenoxazinyl, 2-, 3-, 4-, 5-, 6- or 1-, 3-, 4-, 5-, 6-, 7-, 8-, 9- or 10-benzisoquinolinyl, 2-, 3-, 4 or 5-thieno[2,3-b]furanyl, 2-, 3-, 5-, 6-, 7-, 8-, 9-, 10- or 11-7H-pyrazino[2,3-c]carbazolyl, 2-, 3-, 5-, 6- or 7-2H-furo[3,2-b]-pyranyl, 2-, 3-, 4-, 5-, 7- or 8-5H-pyrido[2,3-d]-o-oxazinyl, 1-, 3- or 5-1H-pyrazolo[4,3-d]-oxazolyl, 2-, 4- or 5-4H-imidazo[4,5-d]thiazolyl, 3-, 5- or 8-pyrazino[2,3-d]pyridazinyl, 2-, 3-, 5- or 6-imidazo[2,1-b]thiazolyl, 1-, 3-, 6-, 7-, 8- or 9-furo[3,4-c]cinnolinyl, 1-, 2-, 3-, 4-, 5-, 6-, 7-, 8-, 9-, 10 or 11-4H-pyrido[2,3-c]carbazolyl, 2-, 3-, 6- or 7-imidazo[1,2-b][1,2,4]triazinyl, 7-benzo[b]thienyl, 2-, 4-, 5-, 6- or 7-benzoxazolyl, 3-, 4-, 5-, 6- or 7-benzisoxazolyl, 4- or 5-(2,1,3-benzisoxadiazolyl), 2-, 4-, 5-, 6- or 7-benzimidazolyl, 2-, 4-, 5-, 6- or 7-benzothiazolyl, 2-, 3-, 4-, 5-, 6- or 7-benzo[b]furanyl, 1-, 2-, 4-, 5-, 6-, 7-, 8- or 9-benzoxapinyl, 2-, 4-, 5-, 6-, 7- or 8-benzoxazinyl, 1-, 2-, 3-, 5-, 6-, 7-, 8-, 9-, 10- or 11-1H-pyrrolo[1,2-b][2]benzazapinyl. Typical fused heteroaryl groups include, but are not limited to 2-, 3-, 4-, 5-, 6-, 7- or 8-quinolinyl, 1-, 3-, 4-, 5-, 6-, 7- or 8-isoquinolinyl, 2-, 3-, 4-, 5-, 6- or 7-indolyl, 2-, 3-, 4-, 5-, 6- or 7-benzo[b]thienyl, 2-, 4-, 5-, 6- or 7-benzoxazolyl, 2-, 4-, 5-, 6- or 7-benzimidazolyl, 2-, 4-, 5-, 6- or 7-benzothiazolyl.

“Heteroaralkyl” means an heteroaryl group covalently attached to a (C₁-C₆)alkylene group, both of which are defined herein. Heteroaralkyl may be optionally substituted as provided for (C₁-C₆)alkyl. Examples of heteroaralkyl groups include pyridin-3-ylmethyl, 3-(benzofuran-2-yl)propyl, 1,3-thiazolylmethyl, isoxazolylmethyl, 1,2,4-triazolylmethyl, pyridinylmethyl, pyrimidinylmethyl or pyrazinylmethyl and the like.

“Haloalkyl” means alkyl substituted with one or more same or different halo atoms, e.g., —CH₂Cl, —CF₃, —CH₂CF₃, CH₂CCl₃, and the like.

“Optionally substituted” means that the group at issue is optionally substituted as provided herein.

1. Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, if the R₂ₐ and R_{2c} substituents in a compound of Formula (I) are attached to the same carbon and are different, then the carbon to which they are attached is an asymmetric center and the compound of Formula (I) can exist as an (R)- or (S)-stereoisomer relative to that carbon. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counterion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.
1. A “pharmaceutically acceptable salt” of a compound means a salt that is pharmaceutically acceptable and that possesses the desired pharmacological activity of the parent compound. Such salts include:
   acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or
2. salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

In particular, compounds of the invention may form internal salts or zwitterions, and these form a particular aspect of the invention. Thus, for example, whilst the compounds are drawn and referred to in say the hydroxyl form, they may exist also in internal salt (zwitterionic) form. The representation of formula (I) and the examples of the present invention covers both hydroxyl and zwitterionic forms and mixtures thereof in all proportions.

“Leaving group” has the meaning conventionally associated with it in synthetic organic chemistry i.e., an atom or group capable of being displaced by a nucleophile and includes halogen (such as chloro, bromo, iodo), alkanesulfonyloxy (such as mesyloxy or trifluorosulfonyloxy) or arenesulfonyloxy (such as tosyloxy), and the like. Leaving Groups are well known in the art and are catalogued in “Protective Groups in Organic Synthesis 3^{rd} Ed.”, edited by Theodora Green and Peter Wuts (John Wiley, 1999).

The compounds of Formula (I) may be administered in the form of a pro-drug which is broken down in the human or animal body to give a compound of the Formula (I). A “Pro-drug” is any compound which releases an active parent drug according to Formula (I) in vivo when such prodrug is administered to a mammalian subject. Prodrugs of a compound of Formula (I) are prepared by modifying functional groups present in the compound of Formula (I) in such a way that the modifications may be cleaved in vivo to release the parent compound. Examples of prodrugs include, but are not limited to esters (e.g., acetate, formate, and benzoate derivatives), carbamates (e.g., N,N-dimethylaminocarbonyl) of hydroxy functional groups in compounds of Formula (I); or esters of carboxy functional groups in compounds of formula I; and the like.

Various forms of pro-drugs are known in the art. For examples of such pro-drug derivatives, see:
1. Design of Prodrugs, edited by H. Bundgaard, (Elsevier, 1985) and Methods in Enzymology, Vol. 42, p. 309-396, edited by K. Widder, et al. (Academic Press, 1985);
2. A Textbook of Drug Design and Development, edited by Krogsgaard-Larsen and H. Bundgaard, Chapter 5 “Design and Application of Prodrugs”, by H. Bundgaard p. 113-191 (1991);
3. H. Bundgaard, Advanced Drug Delivery Reviews, 8, 1-38 (1992);
4. H. Bundgaard, et al., Journal of Pharmaceutical Sciences, 77, 285 (1988);
5. N. Kakeya, et al., Chem Pharm Bull, 32, 692 (1984);
6. K. Beaumont et. al., Current Drug Metabolism, 4, 461 (2003).

An in-vivo hydrolysable ester of a compound of the formula I containing a carboxy or a hydroxy group is, for example, a pharmaceutically-acceptable ester which is hydrolysed in the human or animal body to produce the parent acid or alcohol. Suitable pharmaceutically-acceptable esters for carboxy include (C₁-C₆)alkyl esters, for example ethyl or isopropyl esters; (C₁-C₆)alkoxymethyl esters for example methoxymethyl, (C₁-C₆)alkanoyloxymethyl esters for example pivaloyloxymethyl, phthalidyl esters, (C₃-C₈)cycloalkoxycarbonyloxy(C₁-C₆)alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the Formula (I) containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

“Treating” or “treatment” of a disease includes:
1. preventing the disease, i.e. causing the clinical symptoms of the disease not to develop in a mammal that may be exposed to or predisposed to the disease but does not yet experience or display symptoms of the disease;
2. inhibiting the disease, i.e., arresting or reducing the development of the disease or its clinical symptoms; or
3. relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The phrase “compound of the invention” means those compounds which are disclosed herein, both generically and specifically.

It is to be understood that certain compounds of the formula I may exist in solvated as well as unsolvated forms such as, for example, hydrated forms. It is to be understood that the invention encompasses all such solvated forms which exhibit an inhibitory effect on a5b1, for example an antiangiogenic effect.

It is also to be understood that certain compounds of the formula I may exhibit polymorphism, and that the invention encompasses all such forms which exhibit an inhibitory effect on a5b1, for example an antiangiogenic effect.

It is also to be understood that the invention relates to all tautomeric forms of the compounds of the formula I which exhibit an inhibitory effect on a5b1, for example an antiangiogenic effect.

### Invention Compounds

We turn now to a compound of formula I.

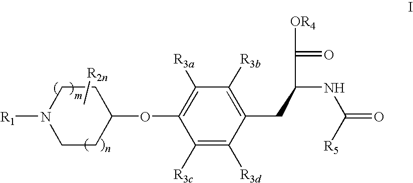

In a compound of formula I, R₁ is an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl. For example, a specific value for R₁ is phenyl. Another specific value for R₁ is benzyl. Other specific values for R₁ include:

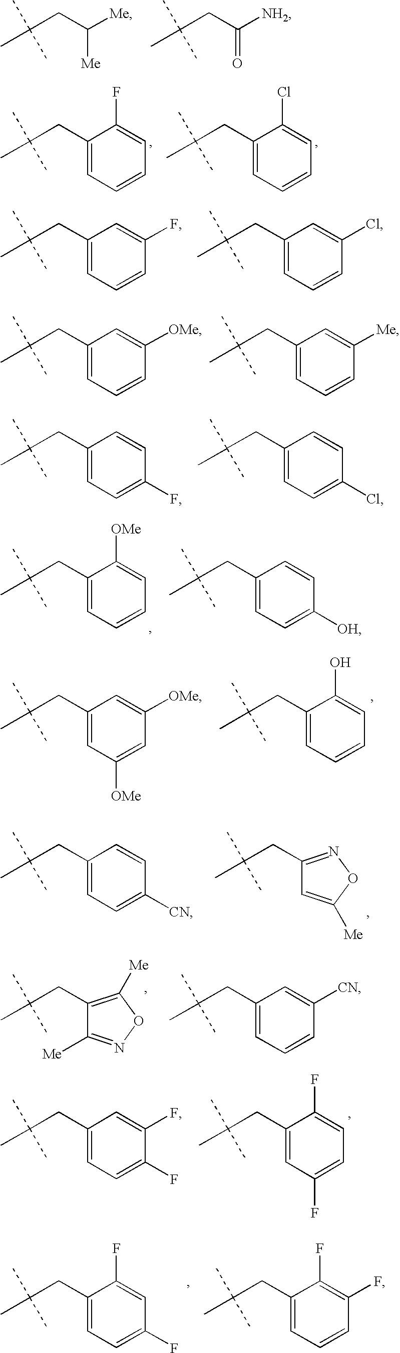

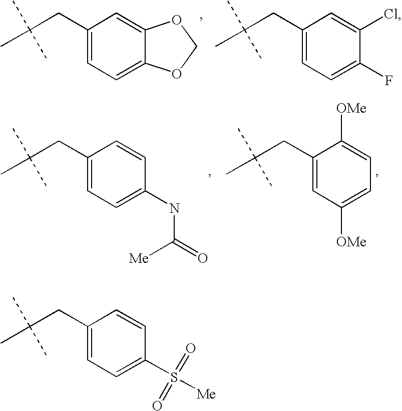

In still other compounds of the invention, R₁ is

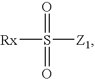

wherein Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl. A specific value for

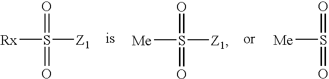

wherein Z₁ is absent. Another specific value for

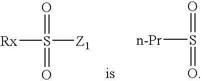

Other specific values for

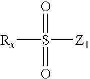

include the following groups:

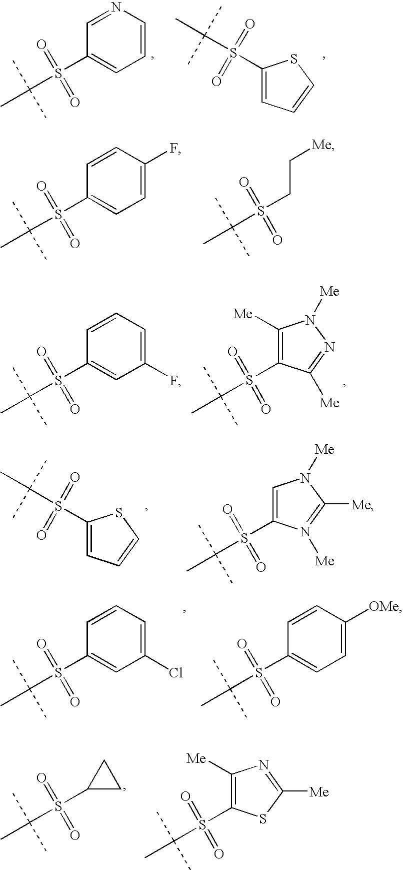

In other compounds of the invention, R₁ is

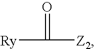

wherein Z₂ is an optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent. R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆) alkylene, heterocyclyl(C₁-C₆)alkyl, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring. For example, a specific value for

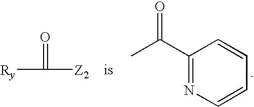

Other specific values for

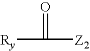

include the following groups:

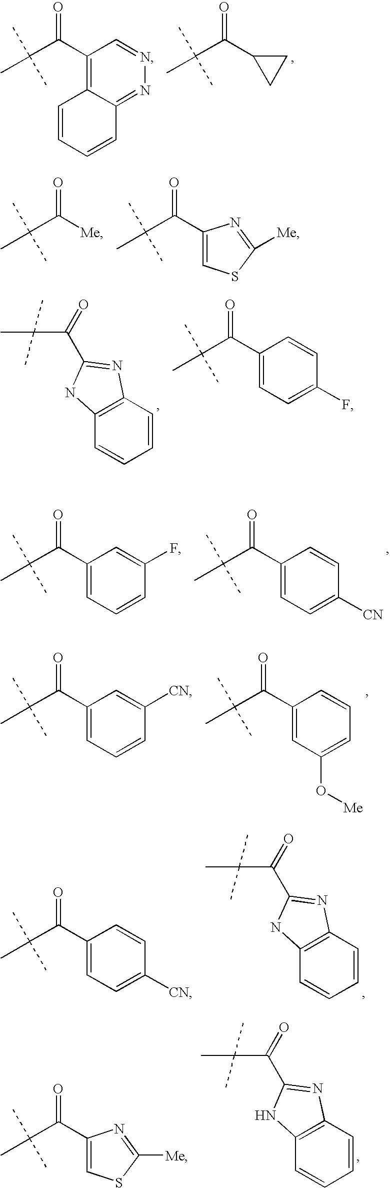

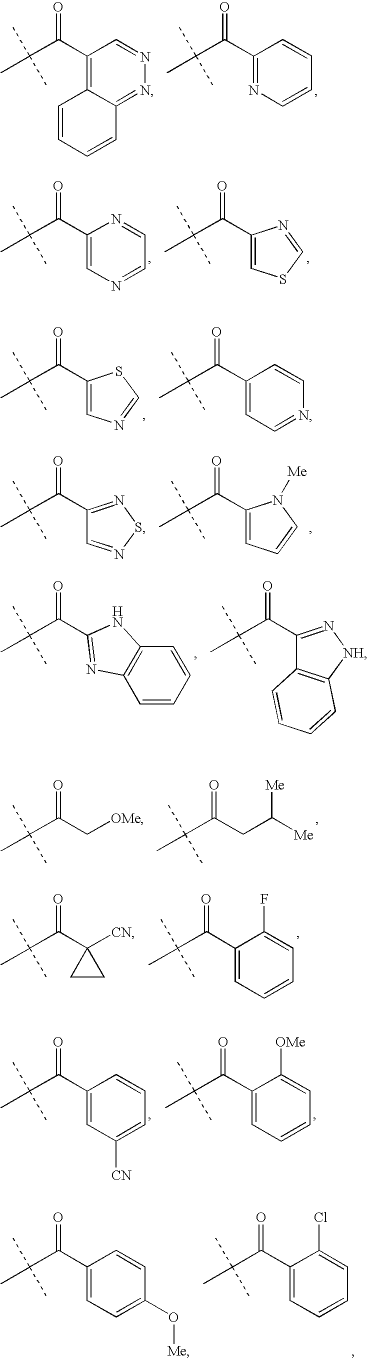

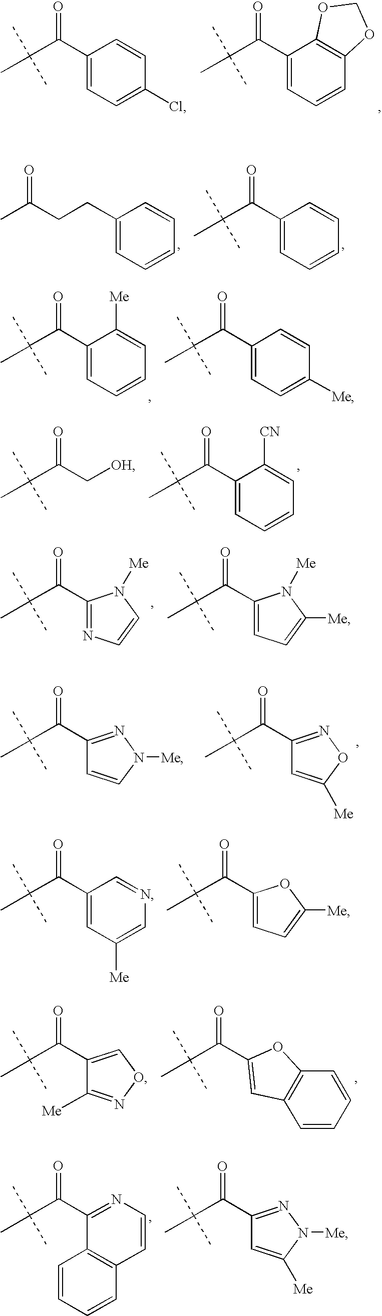

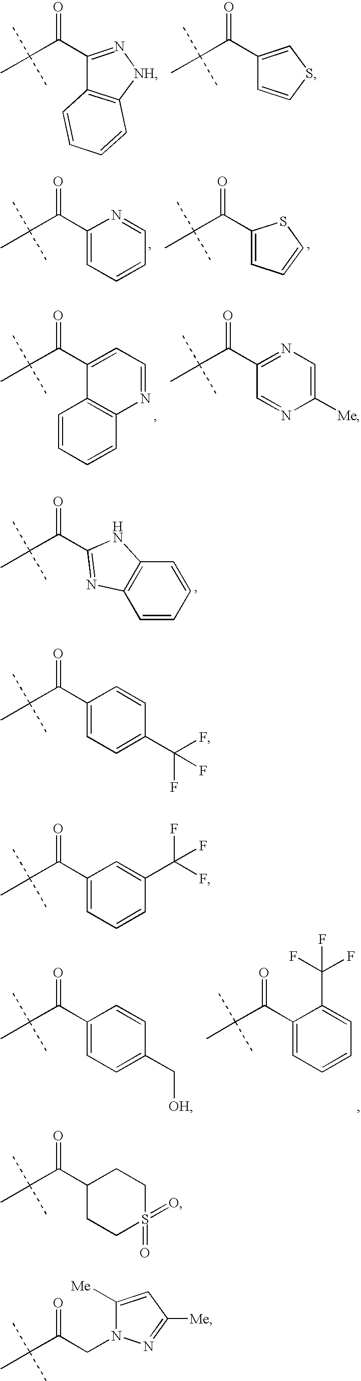

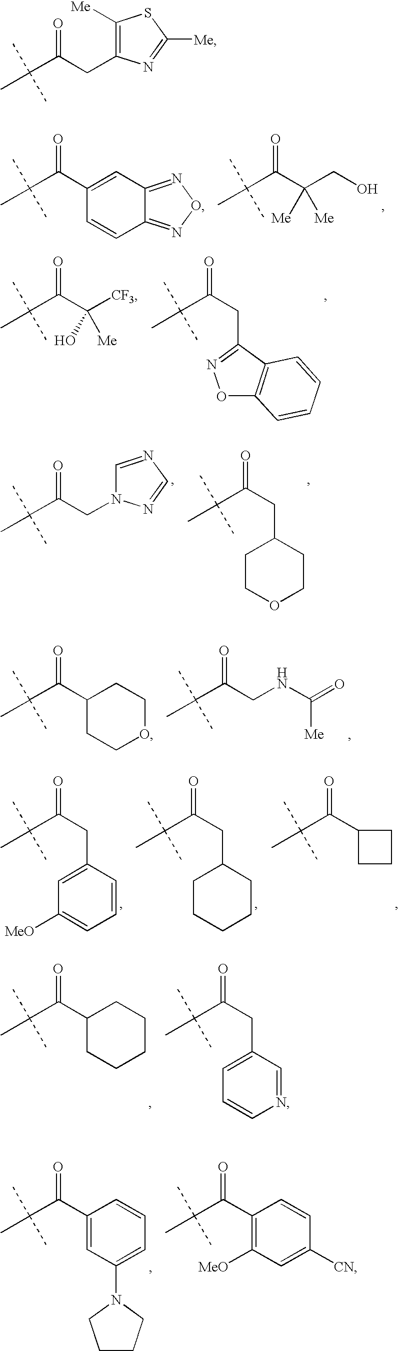

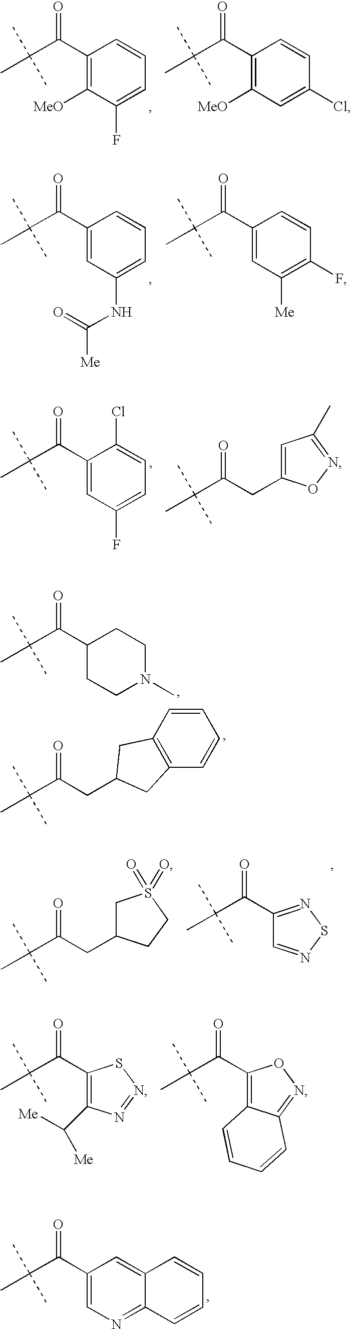

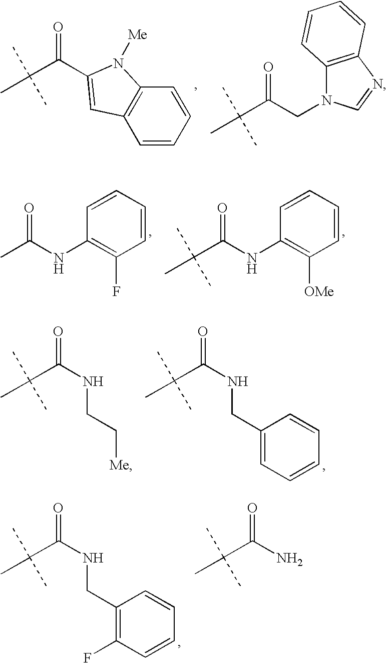

In other compounds of the invention, R₁ is R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring. For example, a specific value for R₁ is

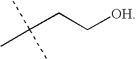

Another specific value for R₁ is

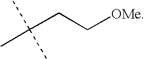

In a compound of formula I, a specific value for

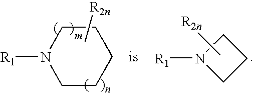

Another specific value for

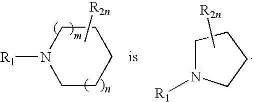

Still other specific values for

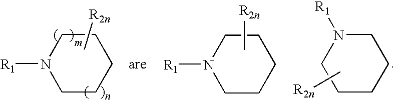

Still other specific values for

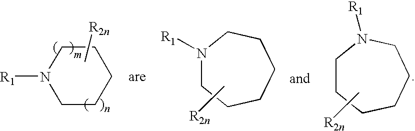

In a compound of formula I, a specific value for

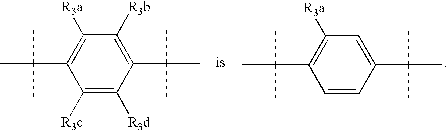

Other specific values for

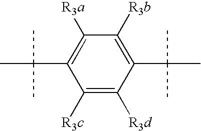

include

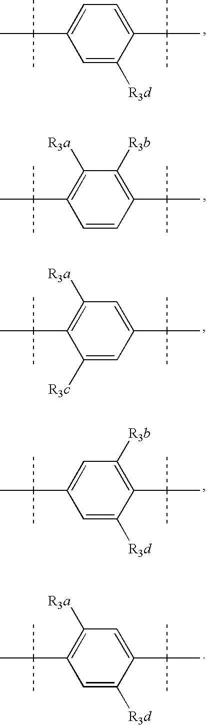

In these specific values, R₃ₐ, R_{3b}, R_{3c}, and R_{3d} are each independently H, halo, (C₁-C₃)alkyl, or (C₁-C₃)alkoxy.

In a compound of formula I, a specific value for R₄ is H. Another specific value for R₄ is Me.

In a particular embodiment of the invention, R

In a compound of formula I, a specific value for R₅ is

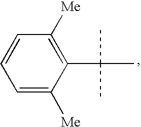

wherein [symbol] indicates the point of attachment. Other specific values for R₅ include

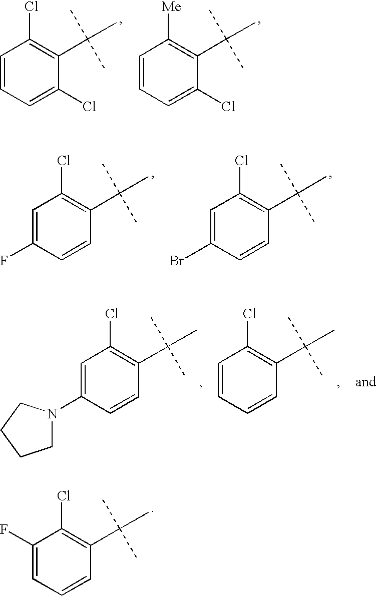

In one embodiment, a compound of formula I is a compound wherein R₄, and R₅ are as provided in the preceding paragraphs and

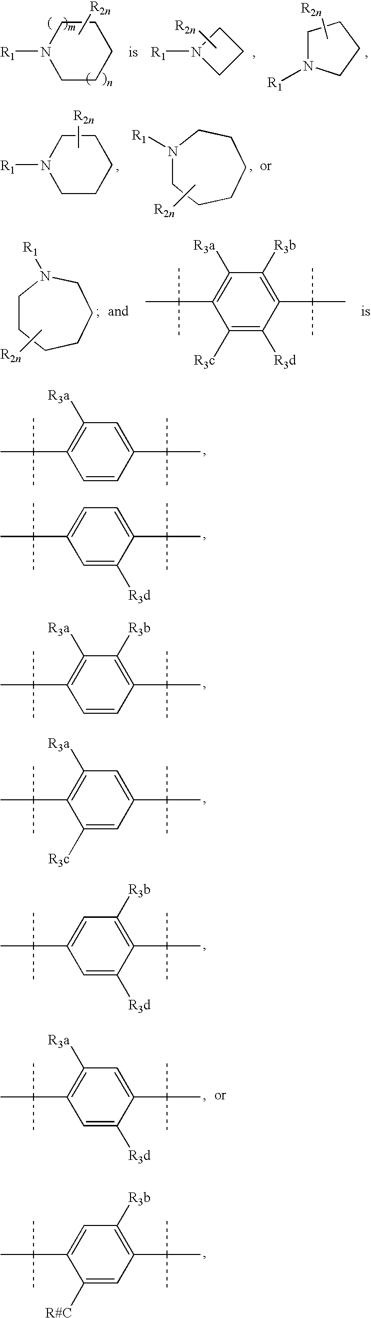

wherein R₃ₐ, R_{3b}, R_{3c}, and R_{3d} are each independently H, halo, (C₁-C₃)alkyl, or (C₁-C₃)alkoxy.

In another embodiment, a compound of formula I is a compound wherein R₄, and R₅ are as provided in the preceding paragraphs and R₂ₙ is two groups, each independently selected from H, halo, hydroxyl, (C₁-C₃)alkyl, or (C₁-C₃)alkoxy, or if two of R₂ₐ and R_{2b} are attached to the same carbon, they may form oxo.

In another embodiment, a compound of formula I is a compound wherein R₁ is an optionally substituted group selected from aralkyl or heteroaralkyl, or is

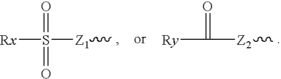

When Rₓ is methyl, R₅ is

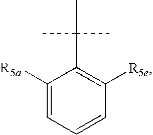

wherein R₅ₐ and R₅ₑ are each independently halo or (C₁-C₃)alkyl.

In another embodiment, a compound of formula I is a compound of formula IA wherein R₁, R₂ₙ, R_{3a-d}, R₄, and R₅ are as defined for a compound of formula I.

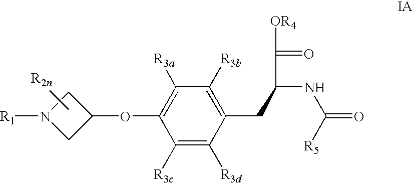

In another embodiment, a compound of formula I is a compound of formula IB, wherein R₁, R₂ₙ, R_{3a-d}, R₄, and R₅ are as defined above.

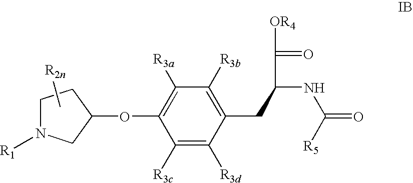

In another embodiment, a compound of formula I is a compound of formula IC-1 or IC-2, wherein R₁, R₂ₙ, R_{3a-d}, R₄, and R₅ are as defined for a compound of formula I.

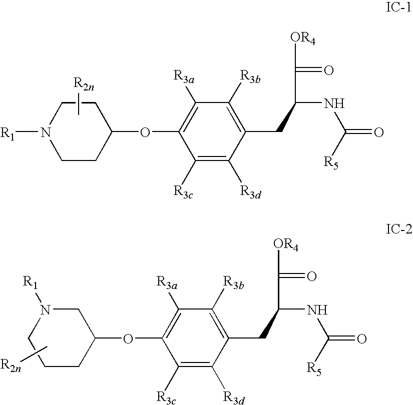

In another embodiment, a compound of formula I is a compound of formula ID-1 or ID-2, wherein R₁, R₂ₙ, R_{3a-d}, R₄, and R₅ are as defined for a compound of formula I.

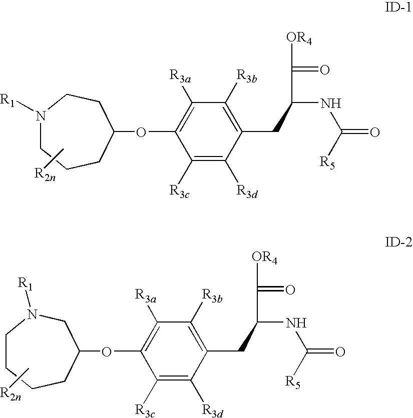

In another embodiment, a compound of the invention is a compound of formula II

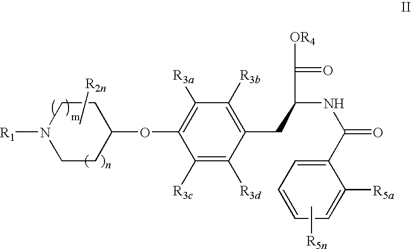

or a pharmaceutical acceptable salt thereof, wherein:
R₁ is
   (a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl;
   (b)

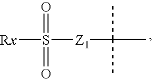

wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl;
(c)

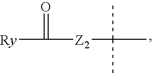

wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₙ is 0, 1, or 2 groups, each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ₐ is halo or (C₁-C₆)alkyl and R₅ₙ is one or two groups selected from halo, (C₁-C₆)alkyl, and (C₁-C₆)alkoxy, provided that when X is N—S(O)₂Me, R₅ is

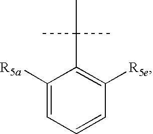

wherein R₅ₐ and R₅ₑ are each independently halo or (C₁-C₃)alkyl.

In another embodiment, a compound of the invention is a compound of formula III

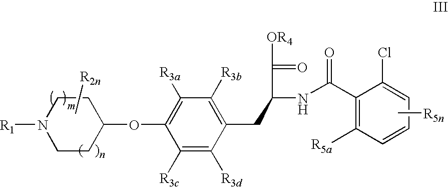

or a pharmaceutical acceptable salt thereof, wherein:
R₁ is
   (a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl;
   (b)

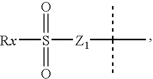

wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl;
(c)

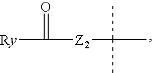

wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₙ is 0, 1, or 2 groups, each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ₛ is H, halo, or (C₁-C₆)alkyl and R₅ₙ is one or two groups selected from halo, (C₁-C₆)alkyl, and (C₁-C₆)alkoxy, provided that when X is N—S(O)₂Me, R₅ is

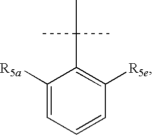

wherein R₅ₐ and R₅ₑ are each independently halo or (C₁-C₃)alkyl.

In another embodiment, a compound of the invention is a compound of formula IV

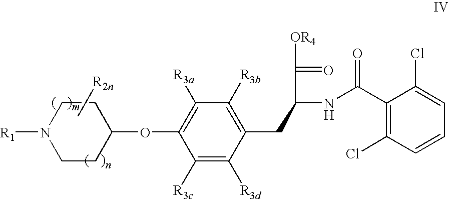

or a pharmaceutical acceptable salt thereof, wherein:
R₁ is
   (a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl;
   (b)

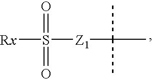

wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl;
(c)

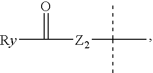

wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆) alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₙ is 0, 1, or 2 groups, each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy; and
R₄ is H, (C₁-C₆)alkyl, aryl, heteroaryl, aralkyl, heteroaralkyl.

Compounds of formula II, III and IV may also be in the form of prodrugs or solvates such as hydrates thereof, if required or convenient.

### Preparation of Invention Compounds

The compounds of the invention may be prepared by various routes, and these may be derived by a skilled person. Particular routes are exemplified in (a) to (h) below.

The present invention also provides that compounds of the formula I, or pharmaceutically acceptable salts thereof, can be prepared by a process (a) to (h) as follows (wherein the variables are as defined above unless otherwise stated):

Process (a) coupling a compound of the formula (V):

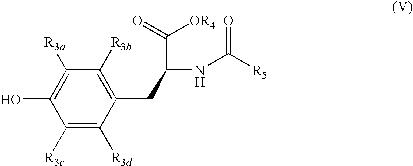

wherein R₃ₐ, R_{3b}, R_{3c}, R_{3d}, R₄ and R₅, are as hereinbefore defined, except any functional group is protected if necessary, and

with a compound of the formula (VI):

Q₁-OH VI

wherein Q₁ is as hereinbefore defined, except any functional group is protected if necessary; or

Process (b) for the preparation of those compounds of formula I wherein either (b′) R₁ is a group of the formula RₓS(O)₂— or (b″) R₈ is a C₁₋₆alkylsulphonyl group which may be optionally substituted as defined above, the reaction, conveniently in the presence of a suitable base, of a compound of the formula I of the formula Ia:

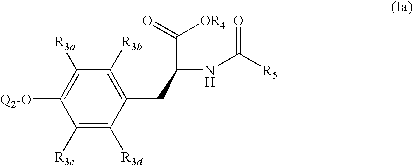

where Q₂ is a group of sub-formula (ai) or (bi)

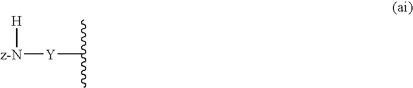

where Y and Z are as defined herein;

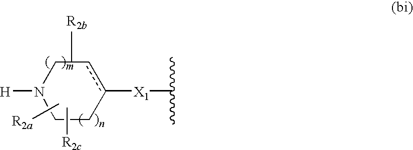

wherein X₁, R₂ₐ, R_{2b}, R_{2c}, m and n are as hereinbefore defined, except any functional group is protected if necessary,

with a compound of the formula VII:

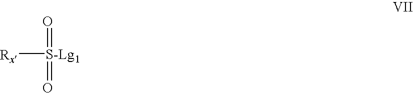

wherein in the case of process (b′) R_{x′} is a group Rₓ is as hereinbefore defined, or in the case of process (b″) R_{x′} an alkyl group optionally substituted by one or more more groups selected from the optional substituents listed above for R₈,
except any functional group is protected if necessary,
and Lg₁ is a leaving group; or
Process (c) for the preparation of those compounds of formula I wherein either (c′) R₁ is a group of the formula R_{y}C(O)— or (c″) R₈ is a (C₂-C₆)alkanoyl group optionally substituted as defined above, the coupling, conveniently in the presence of a suitable base of a compound of the formula I of the formula Ia as hereinbefore defined in relation to Process (b) with a compound of the formula VIII or a reactive derivative thereof:

R_{y′}COOH VIII

where in the case of process (c′), R_{y′} is a group R_{y} as hereinbefore defined, or in the case of (c″) R_{y′} is a C₁₋₅alkyl group optionally substituted by one or more groups selected from the optional substitutents defined for R₈, provided that any reactive groups are optionally protected;
or
Process (d) for the preparation of those compounds of formula I wherein Q₁ is a group of sub-formula (bi) and “- - -” in the compounds of formula I is absent, the reduction of a compound of the formula I wherein “- - -” is a bond; or
Process (e) the coupling of a compound of the formula IX:

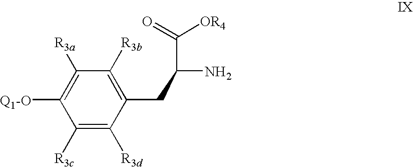

wherein Q₁, R₃ₐ, R_{3b}, R_{3c}, R_{3d}, R₄, X₁, m and n are as hereinbefore defined, except any functional group is protected if necessary,

with a compound of the formula X or a reactive derivative thereof:

R₅COOH X

wherein R₅ is as hereinbefore defined, except any functional group is protected if necessary; or

Process (f) for the preparation of those compounds of formula I wherein either (f′) R₁ is optionally substituted (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aralkyl or heteroaralkyl, or (f″) R₈ is a (C₁-C₆)alkyl group, the reaction, conveniently in the presence of a suitable base, of a compound of the formula Ia as hereinbefore defined in relation to Process (b), with a compound of the formula XI:

R_{1′}-Lg₂ XI

wherein, and

Lg₂ is a suitable leaving group; or

Process (g) for the preparation of those compounds of formula I wherein R₁ or R₈ is a group of the formula R′HNC(O)—, the reaction of a compound of the formula Ia as hereinbefore defined in relation to Process (b) with an isocyanate of the formula XII:

R′N═C(O) XII

wherein R′ is as hereinbefore defined, except any functional group is protected if necessary; or
Process (h) for the preparation of those compounds of formula I wherein R₁ or R₈ is aryl or heteroaryl, the coupling in the presence of a suitable catalyst, of a compound of the formula I of the formula Ia as hereinbefore defined in relation to Process (b) with an aryl or heteroaryl boronic acid, or an ester thereof;

and thereafter, if necessary (in any order):

(i) converting a compound of the formula I into another compound of the formula I;
(ii) removing any protecting groups; and
(iii) forming a pharmaceutically acceptable salt of the compound of formula I.

Specific conditions for the above reactions are as follows.

### Reaction Conditions for Process (a)

The coupling reaction is suitably carried out using the Mitsunobu reaction. Suitable Mitsunobu conditions include, for example, reaction in the presence of a suitable tertiary phosphine and a di-alkylazodicarboxylate in an organic solvent such as an ether, for example THF or a halogenated solvent such as methylene chloride. The reaction is suitably carried out in the temperature range −15° C. to 60° C., for example at or near ambient temperature. A suitable tertiary phosphine includes for example tri-n-butylphosphine or particularly tri-phenylphosphine. A suitable di-alkylazodicarboxylate includes for example diethyl azodicarboxylate (DEAD) or di-tert-butyl azodicarboxylate (DTAD) or azodicarbonyldipiperidine (DPAD). Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

Compounds of formula V are suitably prepared by reacting a compound of formula XIII

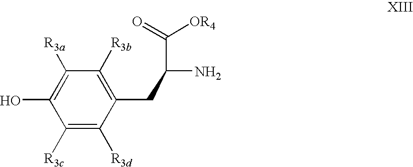

wherein R₁, R₂ₐ, R_{2b}, R_{2c}, R₃ₐ, R_{3b}, R_{3c}, R_{3d}, R₄, X₁, m and n are as hereinbefore defined, except any functional group is protected if necessary, with a compound of formula VIII or a reactive derivative thereof. Suitable reaction conditions are those described for process (c). In particular, a reactive derivative such as an acyl chloride is used in the reaction.

Compounds of formula VI are either known compounds of they may be prepared from known compounds by conventional methods.

### Reaction Conditions for Process (b)

Lg₁ is for example halo such as chloro.

The reaction is advantageously carried out in the presence of base. A suitable base is, for example, an organic amine base such as, for example, pyridine, 2,6-lutidine, collidine, 4-dimethylaminopyridine, triethylamine, diisopropylethylamine, N-methylmorpholine or diazabicyclo[5.4.0]undec-7-ene or an alkali metal or alkaline earth metal carbonate or hydroxide, for example sodium carbonate, potassium carbonate, cesium carbonate, calcium carbonate, sodium hydroxide or potassium hydroxide or MP-carbonate. Alternatively such a base is, for example, an alkali metal hydride, for example sodium hydride, an alkali metal or alkaline earth metal amide, for example sodium amide or sodium bis(trimethylsilyl)amide or a sufficiently basic alkali metal halide, for example cesium fluoride or sodium iodide

The reaction is suitable carried out in an inert solvent such as pyridine.

The reaction is suitable performed at ambient temperature.

Compounds of the formula VII are commercially available or they are known in the literature or they can be prepared by standard processes known in the art.

Compounds of formula (Ia) are suitably prepared using for example the method of process (a) above.

### Reaction Conditions for Process (c)

The coupling reaction may be carried out using standard methods for the coupling of acids and amines. The coupling reaction is conveniently carried out in the presence of a suitable coupling reagent. Standard peptide coupling reagents known in the art can be employed as suitable coupling reagents for example O-(benzotriazol-1-yl)-N,N,N′,N′-tetramethyluronium tetrafluoroborate (TBTU) or O-(7-azabenzotriazol-1-yl)-N,N,N′,N′-tetramethyluronium hexafluoro-phosphate (HATU) or for example carbonyldiimidazole, dicyclohexylcarbodiimide and N-ethyl-N′-(3-dimethylaminopropyl)carbodiimide, optionally in the presence of a catalyst such as dimethylaminopyridine, 4-pyrrolidinopyridine or 2-hydroxy-pyridine-N-oxide, optionally in the presence of a base for example triethylamine, diisopropylethylamine, N-methylmorpholine, pyridine or 2,6-di-alkyl-pyridines such as 2,6-lutidine or 2,6-di-tert-butylpyridine. The reaction is conveniently performed in the present of a suitable inert solvent. Suitable solvents include N,N-dimethylacetamide, dichloromethane, benzene, tetrahydrofuran and N,N-dimethylformamide. The coupling reaction is conveniently performed at a temperature in the range of −40 to 40° C.

A “reactive derivative” of the acid of the formula IX is a carboxylic acid derivative that will react with the amine of the formula Ia to give the corresponding amide. A suitable reactive derivative of a carboxylic acid of the formula IX is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol, an ester such as pentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines is well known in the art, for example they may be reacted in the presence of a base, such as those described above and in a suitable solvent, such as those described above. The reaction may conveniently be performed at a temperature as described above.

Compounds of the formula VIII are commercially available or they are known in the literature or they can be prepared by standard processes known in the art.

### Reaction Conditions for Process (d)

The reduction may be effected by for example hydrogenation over a suitable to catalyst, for example a platinum or palladium on carbon catalyst.

### Reaction Conditions for Process (e)

The coupling may be carried out under analogous conditions to those described above in relation to Process (c) for the coupling of acids and amines. Suitable reactive derivatives of the compound of the formula X are carboxylic acid derivatives such as those described in relation to reactive derivatives of the compound of formula VIII described hereinbefore.

Compounds of the formula IX are novel compounds and as such form a further aspect of the invention. They are suitably prepared by coupling a compound of formula XIII as defined above to a compound of formula VI as defined above. Suitable reaction conditions are those described above in relation to process (a). Preferably at least the amine group in the compound of formula XIII is protected.

Compounds of formula XIII and VI are commercially available or they are known in the literature or they can be prepared by standard processes known in the art.

### Reaction Conditions for Process (f)

Lg₂ is a leaving group for example halo such as chloro or bromo.

The reaction is suitably carried out in the presence of a base, for example one of the bases described in relation to Process (b).

The reaction is suitably carried out in an inert solvent such as acetonitrile.

The reaction is suitably performed at ambient temperature.

### Reaction Conditions for Process (g)

The reaction is suitably carried out in the presence of an inert solvent, for example an ether such as tetrahydrofuran. The reaction is suitably performed at ambient temperature.

### Reaction Conditions for Process (h)

Suitable an aryl or heteroaryl boronic acids for use in this reaction are compounds of the formula R₁B(OH)₂, wherein R₁ is optionally substituted aryl or heteroaryl as defined herein. Esters of boronic acid may also be used, for example compounds of the formula R₁B(OR₉)₂, wherein each R₉ independently is (C₁-C₆)alkyl or the two OR₉ groups together with the boron atom to which they are attached form a ring such as 4,4,5,5-tetramethyl-1,3,2-dioxaborolan-2-yl.

The coupling reaction is suitably performed in the presence of a transition metal catalyst, such as a copper catalyst, for example copper acetate.

The reaction is suitably performed in the presence of a base, for example 2,6-lutidine.

The reaction is conveniently performed in the present of a suitable inert solvent, for example a chlorinated solvent such as dichloromethane. The reaction may be carried out at ambient temperature.

Compounds of the formula I may also be obtained by modifying a substituent in or introducing a substituent into another compound of formula I or a pharmaceutically acceptable salt or prodrug thereof. Suitable chemical transformations are well known to those in the art of organic chemistry. For example, when R⁴ is (1-6C)alkyl in a compound of formula I, the alkyl group may be replaced by hydrogen by hydrolysis of the compound of formula I to give another compound of formula I in which R⁴ is hydrogen. Suitably the hydrolysis is carried out in the presence of a suitable base such as lithium hydroxide. Further representative transformations include the removal of an alkoxycarbonyl group such as tert-butoxycarbonyl, from a compound of the formula I wherein X is NR₁ and R₁ is alkoxycarbonyl. The alkoxycarbonyl group may be removed by treating the compound of formula I with a suitable acid, for example hydrochloric acid.

It will be appreciated that certain of the various ring substituents in the compounds of the present invention may be introduced by standard aromatic substitution reactions or generated by conventional functional group modifications either prior to or immediately following the processes mentioned above and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

When a pharmaceutically acceptable salt of a compound of the formula I is required, for example an acid or base addition salt, it may be obtained by, for example, reaction of the compound of formula I with a suitable acid or base using a conventional procedure. Methods for the preparation of pharmaceutically acceptable salts are well known in the art. For example, the salts may be formed by reacting the free base or free acid form of the product with one or more equivalents of the appropriate acid or base in a solvent or medium in which the salt is insoluble or in a solvent such as water, which is removed in vacuo or by freeze drying or by exchanging the anions of an existing salt for another anion on a suitable ion-exchange resin.

To facilitate isolation of a compound of the formula I during its preparation, the compound may be prepared in the form of a salt that is not a pharmaceutically acceptable salt. The resulting salt can then be modified by conventional techniques to give a pharmaceutically acceptable salt of the compound. Such salt modification techniques are well known and include, for example ion exchange techniques or re-precipitation of the compound from solution in the presence of a pharmaceutically acceptable counter ion as described above, for example by re-precipitation in the presence of a suitable pharmaceutically acceptable acid to give the required pharmaceutically acceptable acid addition salt of a compound of the formula I.

Stereoisomers of compounds of formula I may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions that will not cause racemisation or epimerisation or by derivatisation, with a chiral reagent. When a specific stereoisomer is isolated it is suitably isolated substantially free from other stereoisomers, for example containing less than 20%, particularly less than 10% and more particularly less than 5% by weight of other stereoisomers.

In the synthesis section above the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order and/or the individual reactions may be performed at different stage in the overall route (i.e. chemical transformations may be performed upon different intermediates to those associated hereinbefore with a particular reaction).

Certain intermediates used in the processes described above form a further feature of the present invention. Accordingly there is provided a compound selected from a compound the formula IX as hereinbefore defined or a salt thereof.

In particular, compounds of the invention can be prepared as provided in Scheme 1. In Scheme 1, “P” as used, for example, in the structure

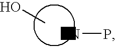

designates a protecting group as found in Greene, which is referenced supra, or as otherwise known to the skilled artisan.

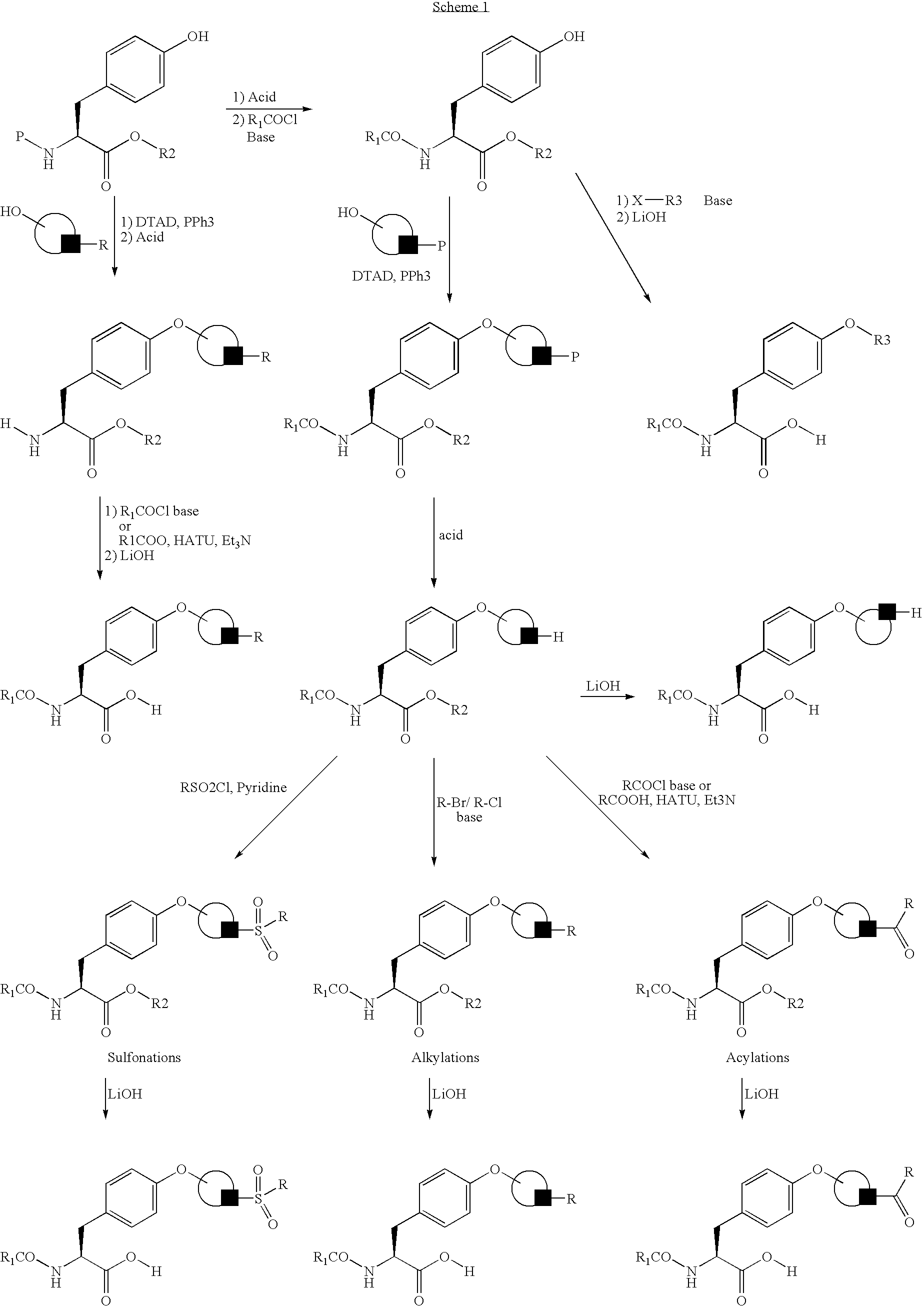

To facilitate isolation of a compound of the formula I during its preparation, the compound may be prepared in the form of a salt that is not a pharmaceutically acceptable salt. The resulting salt can then be modified by conventional techniques to give a pharmaceutically acceptable salt of the compound. Such salt modification techniques are well known and include, for example ion exchange techniques or re-precipitation of the compound from solution in the presence of a pharmaceutically acceptable counter ion as described above, for example by re-precipitation in the presence of a suitable pharmaceutically acceptable acid to give the required pharmaceutically acceptable acid addition salt of a compound of the formula I.

Stereoisomers of compounds of formula I may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions that will not cause racemisation or epimerisation or by derivatisation, with a chiral reagent. When a specific stereoisomer is isolated it is suitably isolated substantially free from other stereoisomers, for example containing less than 20%, particularly less than 10% and more particularly less than 5% by weight of other stereoisomers.

In the synthesis section above the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order and/or the individual reactions may be performed at different stage in the overall route (i.e. chemical transformations may be performed upon different intermediates to those associated hereinbefore with a particular reaction).

### Pharmaceutical Formulations

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of the disease, to slow the progression of the disease or to reduce in patients with symptoms of the disease the risk of getting worse.

For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

### Combinations

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:
1. antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
2. cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
3. agents which inhibit cancer cell invasion (for example metalloproteinase inhibitors like marimastat and inhibitors of urokinase plasminogen activator receptor function);
4. inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
5. antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
6. vascular damaging agents such as Combretastatin A4 and compounds disclosed in International Patent Applications WO 99/02166, WO 00/40529, WO 00/41669, WO 01/92224, WO 02/04434 and WO 02/08213;
7. antisense therapies, for example those which are directed to the targets listed above, such as ISIS 2503, an anti-ras antisense;
8. gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
9. immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assays can be used to measure the effects of the compounds of the present invention as a5b1 integrin inhibitors.

### (a) In Vitro Binding Assay

The assay determined the ability of compounds to inhibit binding of α5β1 integrin to a cognate ligand, a fragment of human fibronectin. The assay used Origen technology (IGEN International) to measure the compound activity. Briefly, α5β1 integrin was coated onto epoxy-paramagnetic beads (Dynal Biotech UK, Bromborough, Wirral, CH62 3QL, UK, Catalogue No 143.02) and biotinylated-fibronectin ligand was coupled to strepatividin labelled BV-Tag-NHS-Ester (BioVeris Corporation, Witney, Oxfordshire, OX28 4GE, UK, Catalogue No JSF396). The ruthenium-labelled BV-Tag emits a electrochemiluminescence signal upon stimulation which is detected by the Origen reader. Thus, interaction of integrin and ligand causes association of bead and tag, and the resulting electrochemiluminescence signal reflects the level of integrin interaction with fibronectin.

12 μg of human α5β1 purified from placenta (Chemicon, Chandlers Ford, Hampshire, SO53 4NF, UK, Catalogue No CC1055-K) was coated onto surface-activated 3 mg of epoxy-paramagnetic beads in PBS and 1M ammonium sulphate following manufacturers instructions at 4° C. for 24 hours. Coated beads were then washed into Assay Buffer (25 mM Hepes, 150 mM NaCl, 1 mM MgCl, 1 mM MnCl, 0.1% Tween, pH7.4) to give a final concentration of 20 □g of α5β1/ml. Immediately prior to the assay, the beads were further diluted ×40 fold in Assay Buffer to give a concentration of 0.5 μg α5β1/ml.

A DNA fragment encoding the domains 9-10 (amino-acids 1325-1509) of human fibronectin (Swiss-Prot Acession No. P02751) was isolated from cDNA libraries using standard molecular biology and PCR cloning techniques. The cDNA fragment was sub-cloned into a pT73.3 expression vector containing a GST-epitope tag (developed at AstraZeneca; Bagnall et al., Protein Expression and Purification, 2003, 27: 1-11). Following expression in E. coli, the expressed protein, termed Fn9-10, was purified using the GST-tag using standard purification techniques. The recombinant Fn9-10 was subsequently biotinylated using a EZ-link Sulfo-NHS-LC-Biotinylation kit (Perbio Science UK Ltd., Cramlington, Northumberland, NE23 1WA, UK, Catalogue No. 21335) and made to give a final concentration of approximately 1 mg/ml. BV-Tag-NHS-Ester was is labelled with streptavidin by incubation at room temperature following manufacturers instructions and buffer-exchanged into PBS to give a concentration of 0.5 mg/ml. Immediately prior to the assay, biotinylated-Fn9-10 and Streptavidin-labelled BV-Tag were diluted in Assay Buffer to give a final concentrations of 0.6 ug/ml and 1.5 ug/ml respectively. The Fn9-10 and BV-Tag solutions were then mixed together in equal volumes and incubated on ice for at least 30 minutes prior to the assay.

Test compounds were prepared as 10 mM stock solutions in DMSO (Sigma-Aldrich Company Ltd, Gillingham, Dorset SP8 4XT Catalogue No. 154938) and serially diluted with 4% DMSO to give a range of test concentrations at ×4 required final concentration. Aliquots (20 μl) of each compound dilution were placed into each well of a 384-well round bottomed polypropylene plate (Matrix Technologies, Wilmslow, Cheshire, SK9 3LP, Catalogue No. 4340 384). Each plate also contained control wells: maximum signal was created using wells containing 20 μl of 4% DMSO, and minimum signal corresponding to no binding was created using wells containing 20 μl of 80 mM EDTA (Sigma Catalogue No. E7889).

For the assay, 20 μl of a5b1-bead suspension and 40 μl of the Fn9-10/BV-Tag preincubated solution were added to each well containing 20 μl of compound or control solution. Assay plates were then incubated at room temperature for a minimum of 6 hours before being analysed on the Origen plate reader. The minimum value was subtracted from all values, and the signal was plotted against compound concentration to generate IC₅₀ data.

In this assay, compounds of the invention typically exhibit IC₅₀ values in the range of 0.18 μM to 30 μM.

### (b) In Vitro Cell Adhesion Assay

The assay determined the ability of compounds to inhibit the a5β1 integrin mediated adhesion of K562 cells to the ligand, a fragment of human fibronectin. The human K562 erythroleukaemia cell line (LGC Promochem, Teddington, Middlesex, UK, Catalogue No. CCL-243) was routinely maintained in RPMI 1640 medium (Sigma-Aldrich Company Ltd, Gillingham, Dorset SP8 4XT, Catalogue No. R0883) containing 10% heat-inactivated foetal calf serum (PAA lab GmbH, Pasching, Austria Catalogue No. PAA-A15-043) and 1% glutamax-1 (Invitrogen Ltd. Paisley, UK Catalogue No. 35050-038) at 37° C. with 5% CO₂ at densities between 1×10⁵ and 1×10⁶ cells/ml.

A DNA fragment encoding the domains 9-10 (amino-acids 1325-1509) of human fibronectin (Swiss-Prot Acession No. P02751) was isolated from cDNA libraries using standard molecular biology and PCR cloning techniques. The cDNA fragment was sub-cloned into a pT7#3.3 expression vector containing a GST-epitope tag (developed at AstraZeneca; Bagnall et al., Protein Expression and Purification, 2003, 27: 1-11), and the fragment termed Fn9-10. Following expression in E. coli, the expressed protein was purified using the GST-tag using standard purification techniques.

For adhesion assay, a 96-well flat bottomed plate (Greiner Bio one ltd., Gloucester GL10 3SX Catalogue No. 655101) was coated overnight at 4° C. with 100 μl of 20 μg/ml Fn9-10 ligand in Dulbecco's PBS (Gibco#14190-94). The plate was then washed twice with 200 μl of PBS and blocked with 100 μl of 3% BSA (SigmaA7888) in PBS for 1 hour at 37° C. The plates were then washed again 3 times with 200 μl of PBS and left empty.

Test compounds were prepared as 10 mM stock solutions in DMSO (Sigma-Aldrich Company Ltd, Gillingham, Dorset SP8 4XT Catalogue No. 154938) and serially diluted with HBSS (Hanks Buffered Salt solution (Gibco Catalogue No. 14170-088)/2% DMSO to give a range of test concentrations at twice required final concentration. Aliquots (50 μl) of each compound dilution were placed into each well of the Fn9-10 coated plates. Each plate also contained control wells: maximum adhesion signal was created using wells containing 50 μl HBSS/2% DMSO, and minimum signal corresponding to no adhesion was created using wells containing 50 μl HBSS/2% DMSO/20 mM EDTA (Sigma Catalogue No. E7889).

The K562 cells were cultured to ˜1×10⁶ cells/ml, and each culture suspension pooled. Cells were centrifuged at 1200 rpm for 2 mins, and the pellets washed with HBSS followed by HBSS/50 mM HEPES (Sigma Catalogue No. H0887). Cell pellets were pooled and resuspended in HBSS/0.4 mM manganese chloride/50 mM HEPES (MnCl; Sigma Catalogue No. M1787) to give a final concentration of 4×10⁶ cells/ml.

The assay was initiated by the addition of 50 μl of cell suspension into each coated well (200,000 cells/well), thus resulting in final desired compound concentration and a final MnCl concentration of 0.2 mM. The plates were incubated for 45 minutes at 37° C. 5% CO₂. After this time, the solution was flicked off as waste, and the remaining cell layer carefully washed twice with 200 μl of PBS, and then fixed with 200 μl of 100% ethanol for 30 minutes.

After fixation, the ethanol was flicked off to waste and 100 μl of 0.1% Crystal violet stain was added to each well, and incubated at ambient temperature for 15 minutes. Excess stain was removed by rinsing ˜3 times under cold slow running water. The plates were blotted over tissue then solubilised by adding 50 μl of 1% Triton X100 (Sigma Catalogue No. T9284) and shaking at 500 rpm for 30 mins on plate shaker. Finally, 100 μl of deionised water was added to each well and the absorbance was determined at 590 nM on a spectrophotometer. The minimum value was subtracted from all values, and the absorbance signal was plotted against compound concentration to generate IC₅₀ data.

In this assay, compounds of the invention typically exhibit IC₅₀ values in the range of 2.4 μM to 30 μM.

Although the pharmacological properties of the compounds of the formula I vary with structural change as expected, in general activity possessed by the compounds of the formula I, may be demonstrated in one or more of the above tests (a) and (b).

Activity data for the following compounds was observed.

| | | Binding | Adhesion |
|---|---|---|---|
| Example | Structure | Assay | Assay |
| 17 | 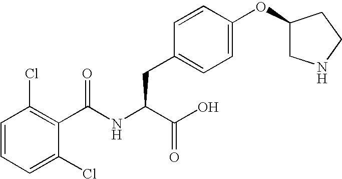 | 1.2 | 9.3 |
| 20 | 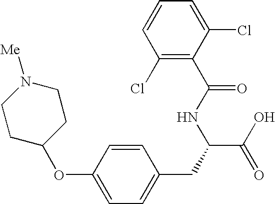 | 0.185 | 2.4 |
| 48 | 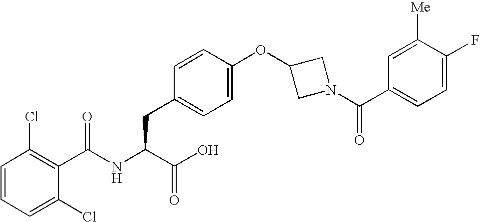 | 0.466 | 4.0 |
| 76 | 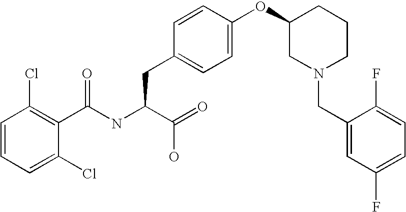 | 4.1 | 11.4 |
| 83 | 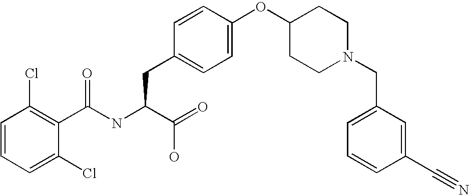 | 1.7 | 6.6 |

The compounds of the present invention are expected to possess, amongst others, anti-angiogenic properties such as anti-cancer properties that are believed to arise from their a5b1 inhibitory properties. The compounds according to the present invention may be useful for the effective treatment of, for example a5b1 driven tumours.

Accordingly, the compounds of the present invention are expected to be useful in the treatment of diseases or medical conditions mediated alone or in part by a5b1 integrin, i.e. the compounds may be used to produce an a5b1 inhibitory effect in a warm-blooded animal in need of such treatment. Thus the compounds of the present invention provide a method for the treatment of malignant cells characterised by inhibition of a5b1. Particularly the compounds of the invention may be used to produce anti-angiogenic and/or an anti-proliferative and/or anti-invasive effect mediated alone or in part by the inhibition of a5b1. Particularly, the compounds of the present invention are expected to be useful in the prevention or treatment of those tumours that are sensitive to inhibition of a5b1 that are involved in for example angiogenesis, proliferation the signal transduction steps which drive proliferation, invasion and particularly angiogenesis of these tumour cells. Accordingly the compounds of the present invention may be useful in the treatment of hyperproliferative disorders, including psoriasis, benign prostatic hyperplasia (BPH), atherosclerosis and restenosis and/or cancer by providing an anti-proliferative effect, particularly in the treatment of a5b1 sensitive cancers. Such benign or malignant tumours may affect any tissue and include non-solid tumours such as leukaemia, multiple myeloma or lymphoma and also solid tumours, for example bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancers. The compounds of the invention are expected to be useful in the treatment of pathogenic angiogenesis, for example in the treatment of cancers as hereinbefore described and other diseases in which inappropriate or pathogenic angiogenesis occurs. The compounds of the invention may also be useful in the treatment or prophylaxis of other conditions in which a5b1 is implicated, for example thrombosis, cardiac infarction, coronary heart diseases, arteriosclerosis, tumours, osteoporosis, inflammations or infections.

In another aspect of the present invention there is provided a compound of formula I or a pharmaceutically acceptable salt, prodrug or hydrate thereof, as defined hereinbefore for use as a medicament.

In another embodiment the present invention provides the use of a compound of formula I or a pharmaceutically acceptable salt, prodrug or hydrate thereof in the preparation of a medicament.

In another embodiment the present invention provides a compound of formula I or a pharmaceutically acceptable salt, prodrug or hydrate thereof for use in the treatment or prophylaxis of a cancer, for example a cancer involving a solid tumor.

In another embodiment the present invention provides a compound of formula I or a pharmaceutically acceptable salt, prodrug or hydrate thereof for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system and other tumor types such as melanoma, multiple myeloma, fibrosarcoma and osteosarcoma and malignant brain tumors.

In still another embodiment the present invention provides a compound of formula I or a pharmaceutically acceptable salt, prodrug or hydrate thereof for use in the treatment or prophylaxis of pathologically angiogenic diseases, thrombosis, cardiac infarction, coronary heart diseases, arteriosclerosis, tumors, osteoporosis, inflammations or infections.

In another embodiment the present invention provides a compound of formula I or a pharmaceutically acceptable salt, prodrug or hydrate thereof for use in the inhibition of a5b1 activity.

In another embodiment the present invention provides a compound of formula I or a pharmaceutically acceptable salt, prodrug or hydrate thereof for use as an antiangiogenic agent in the treatment of a solid tumour.

In another embodiment the present invention provides the use of a compound of formula I or a pharmaceutically acceptable salt, prodrug or hydrate thereof in the preparation of a medicament for the treatment or prophylaxis of a cancer, for example a cancer involving a solid tumour.

In another embodiment the present invention provides the use of a compound of formula I or a pharmaceutically acceptable salt, prodrug or hydrate thereof in the preparation of a medicament for the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system and other tumor types such as melanoma, multiple myeloma, fibrosarcoma and osteosarcoma and malignant brain tumors.

In still another embodiment the present invention provides the use of a compound of formula I or a pharmaceutically acceptable salt, prodrug or hydrate thereof in the preparation of a medicament for the treatment or prophylaxis of pathologically angiogenic diseases, thrombosis, cardiac infarction, coronary heart diseases, arteriosclerosis, tumors, osteoporosis, inflammations or infections.

In another embodiment the present invention provides the use of a compound of formula I or a pharmaceutically acceptable salt, prodrug or hydrate thereof in the preparation of a medicament for use in the inhibition of a5b1 activity.

In another embodiment the present invention provides the use of a compound of formula I or a pharmaceutically acceptable salt, prodrug or hydrate thereof in the manufacture of a medicament for use as an antiangiogenic agent in the treatment of a solid tumour.

In a further aspect of the invention there is provided a pharmaceutical composition which comprises a compound of the formula I or a pharmaceutically acceptable salt, prodrug or hydrate thereof, as defined herein before in association with a pharmaceutically-acceptable diluent or carrier for use in the production of an a5b1 inhibitory effect in a warm-blooded animal such as man.

In a further aspect of the invention there is provided a pharmaceutical composition which comprises a compound of the formula I or a pharmaceutically acceptable salt, prodrug or hydrate thereof, as defined herein before in association with a pharmaceutically acceptable diluent or carrier for use in the production of an anti-cancer effect in a warm-blooded animal such as man.

In a further aspect of the invention there is provided a pharmaceutical composition which comprises a compound of the formula I or a pharmaceutically acceptable salt, prodrug or hydrate thereof, as defined herein before in association with a pharmaceutically acceptable diluent or carrier for use as an antiangiogenic agent in the treatment of a solid tumour.

In a further aspect of the invention there is provided a pharmaceutical composition which comprises a compound of the formula I or a pharmaceutically acceptable salt, prodrug or hydrate thereof, as defined herein before in association with a pharmaceutically-acceptable diluent or carrier for use in the treatment or prophylaxis of pathologically angiogenic diseases, thrombosis, cardiac infarction, coronary heart diseases, arteriosclerosis, tumors, osteoporosis, inflammations or infections.

In another embodiment the present invention provides a method of inhibiting pathogenic angiogenesis in a human or animal comprising administering to said human or animal a therapeutically effective amount of a compound of formula I or a pharmaceutically acceptable salt, prodrug or hydrate thereof.

In a further embodiment the present invention provides a method of inhibiting a5b1 comprising administering to an animal or human in need of said inhibiting a therapeutically effective amount of a compound of formula I or a pharmaceutically acceptable salt, prodrug or hydrate thereof.

In a further embodiment the present invention provides a method of prophylaxis or treatment of a disease mediated in part or alone by a5b1 comprising administering to an animal or human in need of said inhibiting a therapeutically effective amount of a compound of formula I or a pharmaceutically acceptable salt, prodrug or hydrate thereof.

In another embodiment the present invention provides a method of treatment of a human or animal suffering from cancer comprising administering to said human or animal a therapeutically effective amount of a compound of formula I or a pharmaceutically acceptable salt, prodrug or hydrate thereof.

In further embodiment the present invention provides a method of prophylaxis treatment of cancer comprising administering to a human or animal in need of such treatment a therapeutically effective amount of a compound of formula I or a pharmaceutically acceptable salt, prodrug or hydrate thereof.

In another embodiment the present invention provides a method of treatment of a human or animal suffering from a neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system and other tumor types such as melanoma, multiple myeloma, fibrosarcoma and osteosarcoma and malignant brain tumors, comprising administering to said human or animal a therapeutically effective amount of a compound of formula I or a pharmaceutically acceptable salt, prodrug or hydrate thereof.

In another embodiment the present invention provides a method of treatment of a human or animal suffering from a pathologically angiogenic disease, thrombosis, cardiac infarction, coronary heart diseases, arteriosclerosis, tumors, osteoporosis, inflammation or infection, comprising administering to said human or animal a therapeutically effective amount of a compound of formula I or a pharmaceutically acceptable salt, prodrug or hydrate thereof.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter,

100 mm length) using decreasingly polar mixtures as eluent, for example decreasingly polar mixtures of water (containing 1% acetic acid or (NH₄)₂CO₃ 4 g/l) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) the following abbreviations have been used:—

DMF N,N-dimethylformamide

DMSO dimethylsulphoxide

THF tetrahydrofuran

DMA N-dimethylacetamide

DCM Dichloromethane

HATU O-(7-Azabenzotriazol-1-yl)-N,N,N′,N′-Tetramethyluronium Hexafluoro-Phosphate

DTAD di-tert-butyl azodicarboxylate

### Example 1

### Methyl N-(2,6-Dichlorobenzoyl)-L-tyrosinate

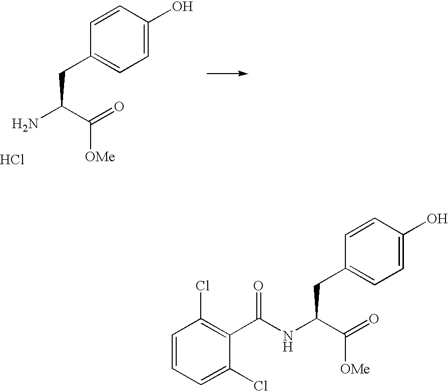

Methyl L-tyrosinate hydrochloride (10 g) was suspended in DCM (200 ml) at 4° C. and triethylamine (13.2 ml) was added as a single portion. 2,6-dichlorobenzoyl chloride (6.18 ml) was added dropwise (T<9° C.) and the reaction stirred overnight at room temperature. The solution was washed with water and brine. Upon standing, a solid precipitate formed from the aqueous layer. The precipitate was filtered, washed with water, and dried to give the title compound as a white solid (4 g). The organic layer was dried and concentrated to give an off-white solid (12 g). The two solids were combined and triturated with a mixture of ethyl acetate/ether/iso-hexane, filtered, and dried to give the title compound as a white solid (12.5 g, 79%).

¹H NMR Spectrum (DMSO-d6) 2.85 (1H, dd), 3.0 (1H, dd), 3.65 (3H, s), 4.65 (1H, m), 6.65 (2H, d), 7.05 (2H, d), 7.45 (3H, m), 9.10 (1H, d), 9.15 (1H, s).

Mass Spectrum [M+H]⁺=368

### Example 2

### Methyl O-(3-chloropropyl)-N-(2,6-dichlorobenzoyl)-L-tyrosinate

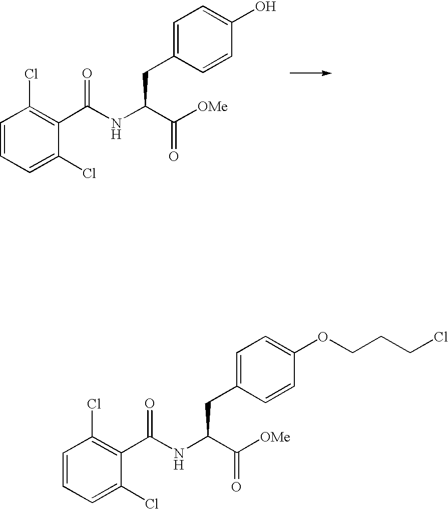

Methyl N-(2,6-dichlorobenzoyl)-L-tyrosinate (3.0 g) was dissolved in DMF (30 ml) and potassium carbonate (3.38 g) added. The mixture was stirred for 1.5 hours before addition of 1-chloro-3-bromopropane (0.97 ml). The reaction was stirred for 18 hours at room temperature. The solvent was removed in vacuo and the resultant yellow oil dissolved in DCM (60 ml) and water (40 ml) added. The organic phase was separated and the aqueous layer was washed with DCM (3×50 ml). The combined organic layers were dried and the solvent removed to afford the title compound as a yellow oil (3.36 g, 93%).

¹H NMR Spectrum (CDCl₃) 2.13 (2H, m), 3.13 (2H, m), 3.65 (5H, m), 3.99 (2H, m), 5.07 (1H, m), 6.18 (1H, m), 6.75 (2H, d), 7.03 (2H, d), 7.20 (3H, m).

Mass Spectrum [M+H]⁺=444

### Example 3

### O-(3-Chloropropyl)-N-(2,6-dichlorobenzoyl)-L-tyrosine

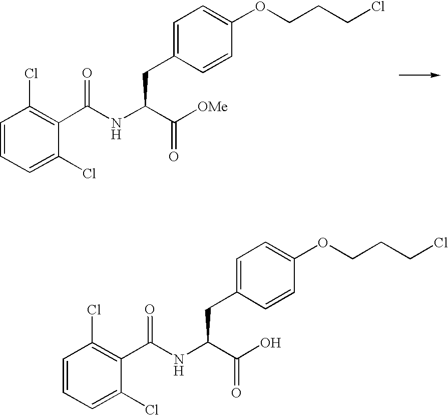

Methyl O-(3-chloropropyl)-N-(2,6-dichlorobenzoyl)-L-tyrosinate (2.94 g) was dissolved in methanol (20 ml) and lithium hydroxide monohydrate (554 mg) was added in one portion. Water (5 ml) was added and the reaction was left to stir at room temperature for 18 hours. The solvent was removed in vacuo and the resulting white solid was dissolved in water (20 ml). This was acidified with 2N HCl (9.9 ml) to give a white precipitate that was filtered, washed with water and dried to afford the title compound as a white solid (2.93 g, 100%).

¹H NMR Spectrum (CDCl₃) 2.13 (2H, m), 3.13 (2H, m), 3.64 (2H, t), 3.97 (2H, m), 5.05 (1H, m), 6.28 (1H, d), 6.73 (2H, m), 7.17 (5H, m).

Mass Spectrum [M+H]⁺=430

### Example 4

### Methyl N-(2,6-dichlorobenzoyl)-O-[3-(methylamino)propyl]-L-tyrosinate

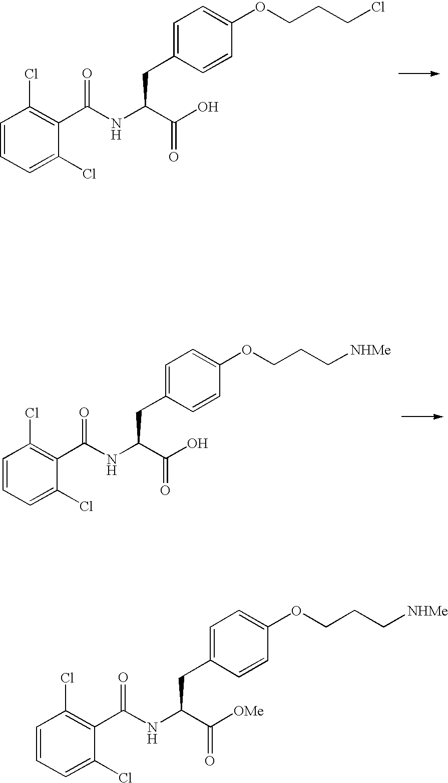

O-(3-Chloropropyl)-N-(2,6-dichlorobenzoyl)-L-tyrosine (1.5 g), methylamine (16 ml, 2M in THF) and tetra-n-butylammonium iodide (858 mg) were mixed and heated in a microwave at 120° C. for 20 minutes. The solution was then concentrated in vacuo and the residue dissolved in methanol (50 ml), and concentrated sulfuric acid (1 ml) was added slowly. The reaction was left stirring overnight to afford an orange solution, then concentrated and the pH adjusted to 8 with methanolic ammonia. The solvent was removed and the residue dissolved in methanol and concentrated. The resulting solid was dissolved in ethyl acetate and washed with water. The organic layer was dried and the concentrated in vacuo to afford an oil which was purified by chromatography using 0-15% methanolic ammonia in DCM as eluent to afford the title compound as a white solid (266 mg, 17%).

¹H NMR Spectrum (CDCl₃) 2.00 (2H, m), 2.47 (3H, s), 2.84 (2H, t), 3.13 (2H, d), 3.64 (3H, s), 3.94 (2H, t), 5.07 (1H, m), 6.21 (1H, d), 6.73 (2H, d), 7.04 (2H, d), 7.20 (3H, m).

Mass Spectrum [M+H]⁺=439

### Example 5

### Methyl N-(2,6-dichlorobenzoyl)-O-[(3R)-pyrrolidin-3-yl]-L-tyrosinate

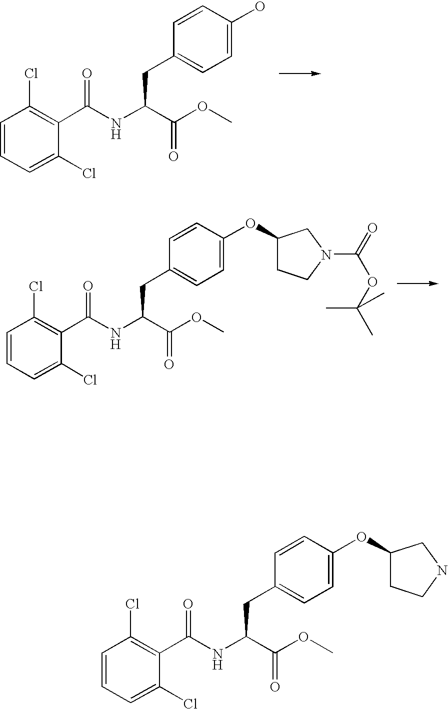

Triphenylphosphine (4.28 g), methyl N-(2,6-dichlorobenzoyl)-L-tyrosinate (3.0 g) and tert-butyl (3S)-3-hydroxypyrrolidine-1-carboxylate (3.06 g) were dissolved in anhydrous DCM (50 ml) under argon and di-tert-butyl azodicarboxylate (3.76 g) in DCM (10 ml) added dropwise at room temperature over 30 minutes. The reaction was stirred for 18 hours then concentrated in vacuo and the residue purified by chromatography using 0 to 10% ethyl acetate in DCM as eluent. The resulting oil and was dissolved in DCM (40 ml) and TFA (10 ml) added. The reaction was stirred at room temperature for 2 hours then concentrated in vacuo. The residue was partitioned between ethyl acetate (150 ml), methanol (15 ml) and saturated sodium carbonate solution (150 ml). The organic layer was separated, washed with water (50 ml×2), brine (50 ml) and dried to afford an oil. DOWEX 50WX4-200R ion exchange resin (100 g) was added to this oil dissolved in methanol (200 ml) and the mixture was stirred slowly for 1 hour, before filtration. The resin was then washed with more methanol (200 ml). The resin was suspended in 7N ammonia in methanol (300 ml) and stirred slowly for 1 hour. The suspension was filtered and the resin washed with more methanolic ammonia (150 ml). Evaporation of the filtrate gave a yellow oil that was triturated with acetonitrile to give the title compound as a white solid (1.32 g, 37%).

NMR Spectrum (CDCl₃) 2.10-2.40 (2H, m), 3.15 (2H, m), 3.50 (4H, m), 3.70 (3H, s), 4.90 (1H, m), 5.10 (1H, m), 6.30 (1H, d), 6.70 (2H, d), 7.10 (2H, d), 7.25 (3H, m).

Mass Spectrum [M+H]⁺=437

The procedure described above was repeated using the appropriate N-BOC protected amino alcohol. Thus were obtained the compounds described below:

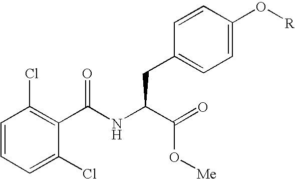

| | | | Mass | NMR Data |
|---|---|---|---|---|
| Example | R | Name | Ion | (300 MHz, CDCl₃₎ |
| 6 | 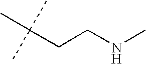 | Methyl N-(2,6- dichlorobenzoyl)- O-[2- (methylamino) ethyl]-l-tyrosinate | 425 [M + H]⁺ | 1.80 (1 H, br), 2.30 (3 H, s), 2.80 (2 H, t), 2.90-3.10 (2 H, m), 3.70 (3 H, s), 4.00 (2 H, t), 4.70 (1 H, m), 6.80 (2 H, d), 7.25 (2 H, d), 7.50 (3 H, m), 9.20 (1 H, d). |
| 7 | 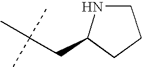 | Methyl N-(2,6- dichlorobenzoyl)- O-[(2S)- pyrrolidin-2- ylmethyl]-L- tyrosinate | 451 [M + H]⁺ | 1.35 (3 H, m), 2.00 (1 H, m), 3.10 (2 H, d), 3.20 (2 H, t), 3.60 (4 H, br s), 3.80 (1 H, m), 4.10 (1 H, m), 5.00 (1 H, m), 6.40 (1 H, d), 6.70 (2 H, d), 7.00 (2 H, d), 7.20 (3 H, m). |
| 8 | 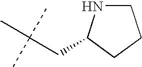 | Methyl N-(2,6- dichlorobenzoyl)- O-[(2R)- pyrrolidin-2- ylmethyl]-L- tyrosinate | 451 [M + H]⁺ | 1.50 (2 H, m), 1.70-2.00 (2 H, m), 2.80-3.00 (2 H, m), 3.15 (2 H, dd), 3.40 (1 H, m), 3.70 (3 H, s), 3.80 (2 H, m), 5.10 (1 H, m), 6.20 (1 H, d), 6.75 (2 H, d), 7.00 (2 H, d), 7.20 (3 H, m). |
| 9 | 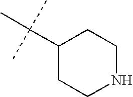 | Methyl N-(2,6- dichlorobenzoyl)- O-piperidin-4-yl- L-tyrosinate | 451 [M + H]⁺ | 1.60 (2 H, m), 2.00 (2 H, m), 2.70 (2 H, m), 3.10 (2 H, m), 3.15 (2 H, d), 3.70 (3 H, s), 4.30 (1 H, m), 5.00 (1 H, m), 6.20 (1 H, d), 6.70 (2 H, d), 7.10 (2 H, d), 7.20 (3 H, m). |

### Alternative Synthesis of Chlorhydrate Intermediates

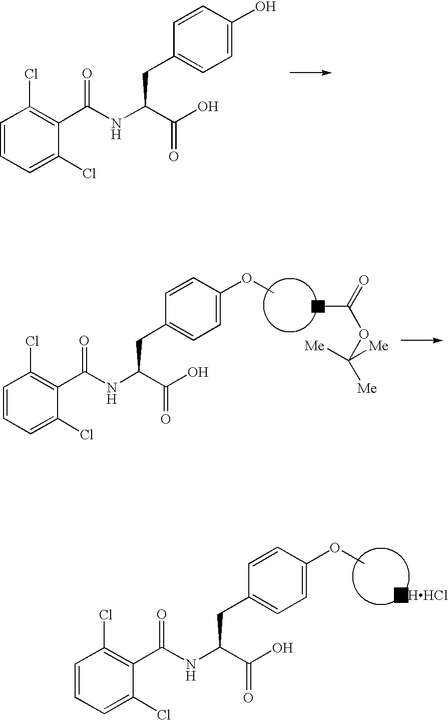

### Example 10

### Mitsunobu Conditions A

Triphenylphosphine (10.4 g, 39.2 mmol, 1.3 equiv.), methyl N-(2,6-dichlorobenzoyl)-L-tyrosinate (10 g, 30.1 mmol, 1 equivalent) and tert-butyl (3R)-3-hydroxypyrrolidine-1-carboxylate (6.75 g, 36.1 mmol, 1.2 equivalent) were dissolved in anhydrous DCM (450 ml) under nitrogen. DTAD (8.3 g, 36.1 mmol, 1.2 equivalent) in DCM (50 ml) was added dropwise at room temperature. After 16 hours of stirring, 0.3 equiv. of the preformed Mitsunobu salt (Triphenylphosphine+DTAD+tert-butyl (3R)-3-hydroxypyrrolidine-1-carboxylate) were added to reach completion.

For azetidine ring-containing systems, the following conditions were used.

### Example 11

### Mitsunobu Conditions B

Triphenylphosphine (10.4 g, 39.2 mmol, 1.3 equiv.), DTAD (8.3 g, 36.1 mmol, 1.2 equiv.) and tert-butyl-3-hydroxy azetidine-1-carboxylate (6.21 g, 36.1 mmol, 1.2 equiv.) were dissolved in anhydrous THF (5 ml) under nitrogen in a sealed tube. Methyl N-(2,6-dichlorobenzoyl)-L-tyrosinate (10 g, 30.1 mmol, 1 equiv.) was added dropwise and the reaction was heated in an oil bath at 110° C. for 5 hours.

The crude mixtures were concentrated in vacuo and the residues purified by chromatography using 30% ethyl acetate in petroleum ether as eluent. The pure BOC intermediates were obtained as foams, and were dissolved in MeOH (15 ml), cooled to 0° C. before a 4N solution of HCl in dioxane (15 ml) was added. The solutions were allowed to stir at room temperature for 3 hours, then were concentrated to dryness to afford the chlorhydrates as white solids (52 to 99% yield).

Thus were obtained the chlorhydrates described below:

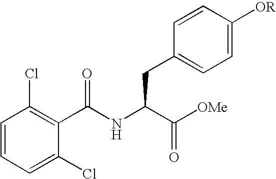

| | | | Mitsunobu | NMR Data |
|---|---|---|---|---|
| Example | R | Name | conditions | (500 MHz, CDCl₃) |
| 10 | 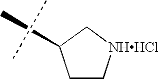 | Methyl N-(2,6- dichlorobenzoyl)- O-[(3R)- pyrrolidin-3-yl]- L-tyrosinate hydrochloride | A | 2.04-2.12 (1 H, m), 2.14- 2.24 (1 H, m), 2.89 (1 H, dd), 3.07 (1 H, dd), 3.32- 3.50 (4 H, m), 3.66 (3 H, s), 4.68 (1 H, ddd), 5.07- 5.12 (1 H, m), 6.89 (2 H, d), 7.23 (2 H, d), 7.41 (1 H, dd), 7.43-7.48 (2 H, m), 9.20 (1 H, d), 9.26 (2 H, bs) |
| 11 | 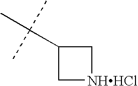 | Methyl N-(2,6- dichlorobenzoyl)- O-azetidin-3-yl- L-tyrosinate hydrochloride | B | 2.89 (1 H, dd), 3.08 (1 H, dd), 3.66 (3 H, s), 3.89- 3.98 (2 H, m), 4.38-4.48 (2 H, m), 4.69 (1 H, ddd), 4.99-5.06 (1 H, m), 6.78 (2 H, d), 7.24 (2 H, d), 7.41 (1 H, dd), 7.43-7.47 (2 H, m), 8.18 (1 H, bs), 8.19 (1 H, d), 9.28 (1 H, bs) |
| 12 | 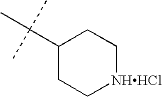 | Methyl N-(2,6- dichlorobenzoyl)- O-piperidin-4-yl- L-tyrosinate hydrochloride | A | This chlorhydrate is present under two protonated pseudo- diastereoisomeric forms in a 60/40 ratio. 1.74-1.86 (2 H, m), 2.02-2.12 (2 H, m), 2.83-2.95 (1 H, m), 3.02-3.11 (3 H, m), 3.18- 3.26 (2 H, m), 3.57 (1.2 H, s), 3.65 (1.8 H, s), 4.57- 4.64 (1.4 H, m), 4.69 (0.6 H, ddd), 6.88-6.94 (2 H, m), 7.18-7.24 (2 H, m), 7.36-7.47 (3 H, m), 8.77 (1 H, bs), 8.2 (1 H, bs), 9.05 (0.4 H, d), 9.18 (0.6 H, d) |
| 13 | 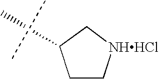 | Methyl N-(2,6- dichlorobenzoyl)- O-[(3S)- pyrrolidin-3-yl]- L-tyrosinate hydrochloride | A | 2.04-2.12 (1 H, m), 2.14- 2.24 (1 H, m), 2.89 (1 H, dd), 3.08 (1 H, dd), 3.20- 3.50 (4 H, m), 3.66 (3 H, s), 4.68 (1 H, ddd), 5.07- 5.13 (1 H, m), 6.89 (2 H, d), 7.23 (2 H, d), 7.41 (1 H, dd), 7.43-7.48 (2 H, m), 9.19 (1 H, d), 9.24 (2 H, bs) |
| 14 | 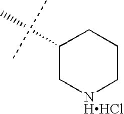 | Methyl N-(2,6- dichlorobenzoyl)- O-[(3R)- piperidin-3-yl]- L-tyrosinate hydrochloride | A | 1.61-1.70 (1 H, m), 1.74- 1.82 (1 H, m), 1.82-1.91 (2 H, m), 2.88 (1 H, dd), 3.01-3.11 (3 H, m), 3.19 (1 H, bs), 3.25-3.35 (1 H, m), 3.66 (3 H, s), 4.63- 4.73 (2 H, m), 6.95 (2 H, d), 7.23 (2 H, d), 7.41 (1 H, dd), 7.43-7.49 (2 H, m), 8.63 (1 H, bs), 8.94 (1 H, bs), 9.19 (1 H, d) |
| 15 | 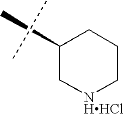 | Methyl N-(2,6- dichlorobenzoyl)- O-[(3S)- piperidin-3-yl]- L-tyrosinate hydrochloride | A | 1.62-1.71 (1 H, m), 1.73- 1.81 (1 H, m), 1.82-1.91 (2 H, m), 2.88 (1 H, dd), 3.01-3.08 (2 H, m), 3.08 (1 H, dd), 3.11-3.18 (1 H, m), 3.25-3.34 (1 H, m), 3.66 (3 H, s), 4.65-4.73 (2 H, m), 6.95 (2 H, d), 7.23 (2 H, d), 7.41 (1 H, dd), 7.43-7.48 (2 H, m), 8.73 (1 H, bs), 9.17 (1 H, bs), 9.18 (1 H, d) |

### Example 16

### N-(2,6-Dichlorobenzoyl)-O-piperidin-4-yl-L-tyrosine

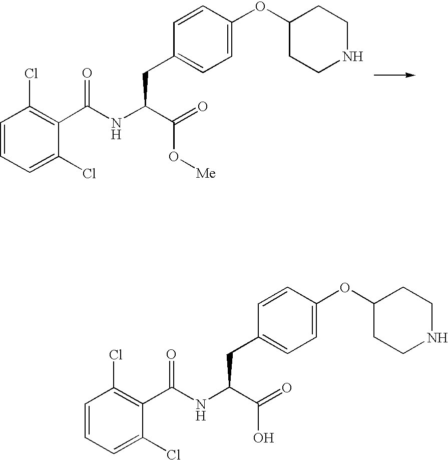

Lithium hydroxide (32 mg) in water (500 μl) was added to a solution of methyl N-(2,6-dichlorobenzoyl)-O-piperidin-4-yl-L-tyrosinate (82 mg) in methanol (1 ml) and acetonitrile (1 ml) and the resulting reaction mixture was stirred overnight. 2N HCl (470 μl) was added and the solution was concentrated in vacuo. The residue was purified by reverse phase chromatography to give the title compound as a solid (22 mg, 28%).

¹H NMR Spectrum (DMSO-d6) 1.80 (2H, m), 2.10 (2H, m), 2.85-2.91 (1H, m), 3.03-3.10 (3H, m), 3.25 (2H, m), 4.59-4.63 (2H, m), 6.91 (2H, d), 7.23 (2H, d), 7.43 (3H, m), 8.92 (1H, d).

Mass Spectrum [M+H]⁺=437

The procedure described above was repeated using the appropriate ester. Thus were obtained the compounds described below:

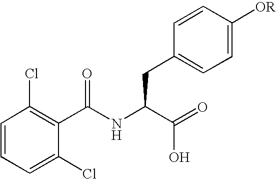

| | | | Mass | | NMR Data |
|---|---|---|---|---|---|
| Example | R | Name | Ion | RT | (400 MHz, DMSO-d₆) |
| 17 | 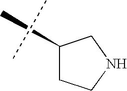 | N-(2,6- Dichlorobenzoyl)- O-[(3R)- pyrrolidin-3- yl]-L-tyrosine | 423 [M + H]⁺ | 1.13 | |
| 18 | 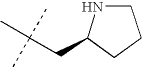 | N-(2,6- Dichlorobenzoyl)- O-[(2S)- pyrrolidin-2- ylmethyl]-L- tyrosine | 437 [M + H]⁺ | 1.06 | 1.76 (1 H, m), 1.94-2.00 (2 H, m), 2.13 (1 H, m), 2.90-2.92 (1 H, m), 3.06- 3.07 (1 H, m), 3.25 (2 H, m), 3.90 (1 H, m), 4.03- 4.07 (1 H, t), 4.22-4.26 (1 H, m), 4.60 (1 H, m), 6.90-6.93 (2 H, m), 7.26 (2 H, m), 7.43 (1 H, t), 7.47 (2 H, m), 8.70 (1 H, br s), 9.06 (1 H, d), 9.20 (1 H, br s) |
| 19 | 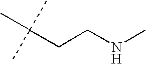 | N-(2,6- dichlorobenzoyl)- O-[2- (methylamino) ethyl]-L- tyrosine | 411 [M + H]⁺ | 0.86 | 2.62 (3 H, s), 2.90-2.93 (1 H, m), 3.07 (1 H, dd), 3.30 (2 H, m), 4.20 (2 H, t), 4.62 (1 H, m), 6.91 (2 H, d), 7.25 (2 H, d), 7.40-7.46 (3 H, m), 8.98 (1 H, d) |

### Example 20

### N-(2,6-Dichlorobenzoyl)-O-(1-methylpiperidin-4-yl)-L-tyrosine

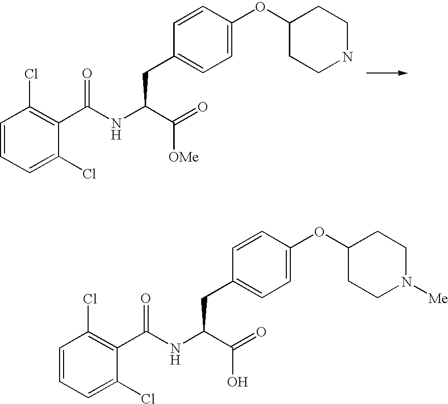

Formaldehyde (0.41 ml, 12.8N), formic acid (0.26 ml) and methyl N-(2,6-dichlorobenzoyl)-O-piperidin-4-yl-L-tyrosinate (82 mg) were heated at 80° C. for 16 hours. The reaction was concentrated in vacuo and the residue dissolved in methanol (1 ml). Lithium hydroxide (32 mg) in water (0.5 ml) was added and the reaction stirred was overnight. The solution was concentrated in vacuo and the residue purified by reverse phase chromatography to give the title compound as a solid (34 mg, 41%).

¹H NMR Spectrum (DMSO-d6) 1.64-1.67 (2H, m), 1.95 (2H, m), 2.29 (3H, s), 2.33-2.35 (2H, m), 2.70 (2H, m), 2.85 (1H, m), 3.05 (1H, m), 4.34 (1H, m), 4.59 (1H, m), 6.86 (2H, d), 7.20 (2H, d), 7.39-7.46 (3H, m), 8.85 (1H, d).

Mass Spectrum [M+H]⁺=451

The procedure described above was repeated using the appropriate amine. Thus were obtained the compounds described below:

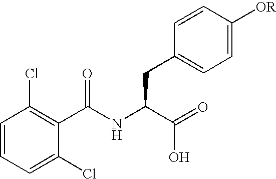

| | | | Mass | | NMR Data |
|---|---|---|---|---|---|
| Example | R | Name | Ion | RT | (400 MHz, DMSO-d₆) |
| 21 | 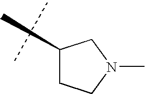 | N-(2,6- Dichlorobenzoyl)- O-[(3R)-1- methyl pyrrolidin-3-yl]- L-tyrosine | 437 [M + H]⁺ | 0.97 | 2.00 (1 H, m), 2.40 (1 H, m), 2.70 (3 H, s), 3.06-3.11 (1 H, m), 3.31 (4 H, m), 4.65 (1 H, m), 5.05 (1 H, m), 6.86 (2 H, d), 7.23 (2 H, m), 7.41- 7.45 (3 H, m), 9.05 (1 H, d) |
| 22 | 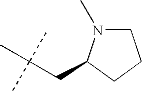 | N-(2,6- Dichlorobenzoyl)- O-{[(2S)-1- methylpyrrolidin- 2-yl]methyl}-L- tyrosine | 451 [M + H]⁺ | 1.07 | |

### Example 23

### N-(2,6-Dichlorobenzoyl)-O-(1-acetylpiperidin-4-yl)-L-tyrosine

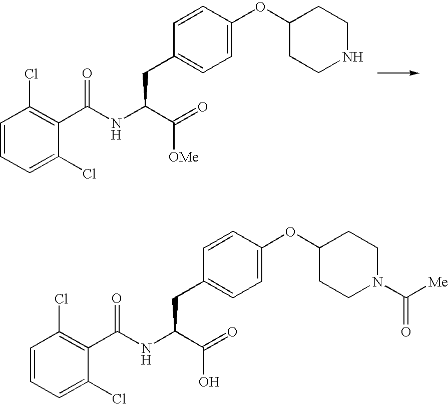

HATU (86 mg) was added to a solution of methyl N-(2,6-dichlorobenzoyl)-O-piperidin-4-yl-L-tyrosinate (85 mg), acetic acid (11 μl) and triethylamine (32 μl) in DMF (2 ml) and stirred overnight. The resulting reaction mixture was concentrated in vacuo and the residue partitioned between ethyl acetate and water, dried and concentrated. The residue was dissolved in methanol (2 ml) and a solution of lithium hydroxide (32 mg) in water (0.5 ml) was added. The mixture was allowed to stir at room temperature overnight then neutralised with 2N HCl, concentrated in vacuo and the residue purified by reverse phase chromatography to give the title compound as a solid (16 mg, 18%).

¹H NMR Spectrum (DMSO-d6) 1.45 (1H, m), 1.60 (1H, m), 1.85 (1H, m), 1.95 (1H, m), 2.02 (3H, s), 2.84-2.90 (1H, m), 3.05-3.10 (1H, m), 3.26 (1H, m), 3.38 (1H, m), 3.67 (1H, m), 3.85 (1H, m), 4.57 (1H, m), 4.65 (1H, m), 6.90 (2H, d), 7.21 (2H, d), 7.40-7.45 (2H, m), 7.44-7.46 (1H, m), 9.02 (1H, d).

Mass Spectrum [M+H]⁺=479

The procedure described above was repeated using the appropriate amine and acid. Thus were obtained the compounds described below:

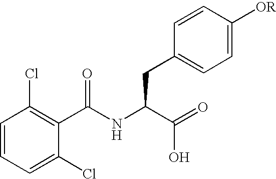

| | | | Mass | NMR Data |
|---|---|---|---|---|
| Example | R | Name | Ion | (400 MHz, DMSO-d₆) |
| 24 | 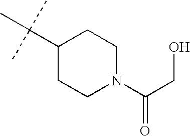 | N-(2,6- dichlorobenzoyl)-O-(1- glycoloylpiperidin-4- yl)-L-tyrosine | 495 [M + H]⁺ | |
| 25 | 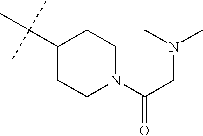 | N-(2,6- Dichlorobenzoyl)-O- [1-(N,N- dimethylglycyl)piperidin- 4-yl]-L-tyrosine | 522 [M + H]⁺ | |
| 26 | 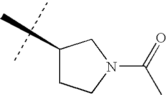 | O-[(3R)-1- Acetylpyrrolidin-3- yl]-N-(2,6- dichlorobenzoyl)-L- tyrosine | 465 [M + H]⁺ | 1.94 (3 H, s), 2.00- 2.20 (2 H, m), 2.90 (1 H, d), 3.07-3.10 (1 H, m), 3.51 (2 H, m), 3.54-3.80 (2 H, m), 4.65 (1 H, m), 5.00- 5.10 (1 H, m), 6.87 (2 H, t), 7.21-7.24 (2 H, m), 7.38-7.44 (3 H, m), 9.01 (1 H, d) |
| 27 | 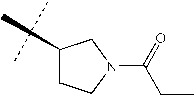 | N-(2,6- Dichlorobenzoyl)-O- [(3R)-1- glycoloylpyrrolidin- 3-yl]-L-tyrosine | 481 [M + H]⁺ | 2.00-.2.25 (2 H, m), 2.91 (2 H, s), 3.10 (1 H, m), 3.47-3.49 (1 H, m), 3.58 (1 H, m), 3.70 (1 H, m), 3.98-4.05 (2 H, d), 4.59 (2 H, m), 5.00-5.10 (1 H, m), 6.85-6.88 (2 H, m), 7.21-7.23 (2 H, m), 7.37-7.45 (3 H, m), 8.90 (1 H, d) |
| 28 | 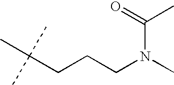 | O-{3- [Acetyl(methyl)amino] propyl}-N-(2,6- dichlorobenzoyl)-L- tyrosine | 465 [M + H]⁺ | 1.88-1.90 (1 H, m), 1.96 (3 H, s), 1.98 (1 H, m), 2.80 (1.5 H, s), 2.84- 2.90 (1 H, m), 2.95 (1.5 H, s), 3.04-3.09 (1 H, m), 3.41 (2 H, t), 3.91-3.96 (2 H, m), 4.62-4.65 (1 H, m), 6.83-6.87 (2 H, m), 7.19-7.23 (2 H, m), 7.38-7.46 (3 H, m), 9.02 (1 H, d), 12.69 (1 H, s) |
| 29 | 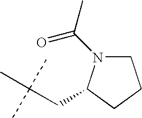 | O-{[(2R)-1- Acetylpyrrolidin-2- yl]methyl}-N-(2,6- dichlorobenzoyl)-L- tyrosine | 477 [M + H]⁺ | 1.89-1.91 (1 H, m), 1.92-1.94 (2 H, m), 1.95 (3 H, s), 2.85- 2.91 (1 H, m), 3.04- 3.08 (1 H, m), 3.42- 3.49 (2 H, m), 3.80- 3.85 (1 H, m), 3.85- 3.95 (1 H, m), 4.05- 4.08 (1 H, m), 4.22 (1 H, d), 4.60-4.63 (1 H, m), 6.86-6.89 (1 H, m), 6.88 (1 H, d), 7.21 (2 H, t), 7.38- 7.46 (3 H, m), 8.99 (1 H, d) |
| 30 | 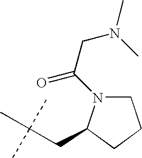 | N-(2,6- Dichlorobenzoyl)-O- {[(2S)-1-(N,N- dimethylglycyl) pyrrolidin-2-yl]methyl}- L-tyrosine | 520 [M + H]⁺ | 1.89-1.94 (3 H, m), 1.96 (1 H, m), 2.35 (6 H, d), 2.85-2.90 (1 H, m), 3.04-3.09 (1 H, m), 3.20-3.38 (2 H, m), 3.45-3.56 (2 H, m), 3.87-3.91 (1 H, m), 4.06-4.09 (1 H, m), 4.26 (1 H, m), 4.61 (1 H, m), 6.88 (2 H, d), 7.21 (2 H, t), 7.40-7.45 (3 H, m), 8.98 (1 H, d) |
| 31 | 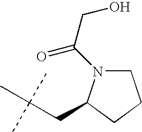 | N-(2,6- Dichlorobenzoyl)-O- {[(2S)-1- glycoloylpyrrolidin- 2-yl]methyl}-L- tyrosine | 495 [M + H]⁺ | 1.92 (4 H, m), 2.90 (1 H, dd), 3.04 (2 H, dd), 3.35 (1 H, d), 3.85- 3.89 (1 H, m), 4.02 (2 H, s), 4.09-4.20 (1 H, m), 4.30 (1 H, m), 4.51 (1 H, m), 4.61 (1 H, m), 6.89 (2 H, m), 7.20 (2 H, d), 7.38- 7.45 (3 H, m), 9.00 (1 H, d) |

### Example 32

### N-(2,6-dichlorobenzoyl)-O-[1-(methylsulfonyl)piperidin-4-yl]-L-tyrosine

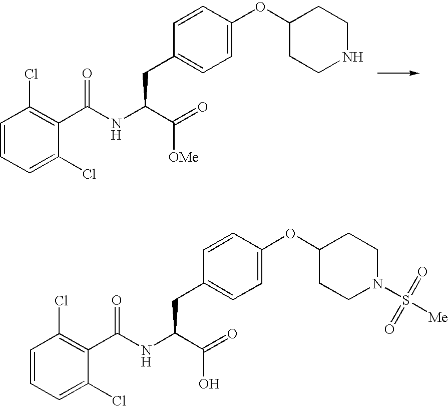

Methyl N-(2,6-dichlorobenzoyl)-O-piperidin-4-yl-L-tyrosinate (85 mg) and 4-dimethylaminopyridine (46 mg) were dissolved in pyridine (1 ml) and to give a clear solution. Methane sulphonyl chloride (25 μl) was added in a single portion and the resulting reaction mixture was stirred at room temperature overnight. The solution was concentrated in vacuo and the residue dissolved in acetonitrile (1 ml) and methanol (1 ml) and a solution of lithium hydroxide (32 mg) in water (0.5 ml) was added. The mixture was stirred at room temperature overnight, then concentrated in vacuo and the residue purified by reverse phase chromatography to give the title compound as a solid (2 mg, 2%).

Mass Spectrum [M+H]⁺=515

The procedure described above was repeated using the appropriate amine. Thus were obtained the compounds described below:

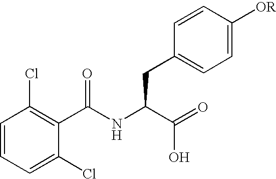

| | | | Mass | NMR Data |
|---|---|---|---|---|
| Example | R | Name | Ion | (400 MHz, DMSO-d₆) |
| 33 | 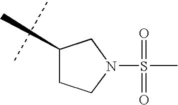 | N-(2,6- Dichlorobenzoyl)-O- [(3R)-1- (methylsulfonyl) pyrrolidin-3-yl]-L- tyrosine | 501 [M + H]⁺ | |
| 34 | 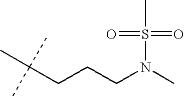 | N-(2,6- Dichlorobenzoyl)-O-{3- [methyl(methylsulfonyl) amino]propyl}-L- tyrosine | 501 [M + H]⁺ | 1.96 (2 H, q), 2.78 (3 H, s), 2.85 (3 H, s), 2.87- 2.91 (1 H, m), 3.04- 3.09 (1 H, m), 3.22 (2 H, t), 3.98 (2 H, t), 4.62-4.65 (1 H, m), 6.84-6.86 (2 H, m), 7.19-7.22 (2 H, m), 7.38-7.46 (3 H, m), 9.03 (1 H, d), 12.69 (1 H, s) |
| 35 | 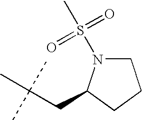 | N-(2,6- Dichlorobenzoyl)-O- {[(2S)-1- (methylsulfonyl) pyrrolidin-2-yl]methyl}- L-tyrosine | 515 [M + H]⁺ | 1.91-2.10 (4 H, m), 2.85-2.91 (1 H, m), 2.97 (3 H, s), 3.04- 3.09 (1 H, m), 3.30 (2 H, m), 3.85 (1 H, t), 3.98-4.03 (2 H, m), 4.62 (1 H, m), 6.87 (2 H, d), 7.21 (2 H, d), 7.38-7.46 (3 H, m), 9.01 (1 H, d) |
| 36 | 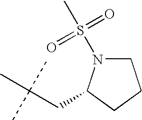 | N-(2,6- Dichlorobenzoyl)-O- {[(2R)-1- (methylsulfonyl) pyrrolidin-2-yl]methyl}- L-tyrosine | 515 [M + H]⁺ | |
| 37 | 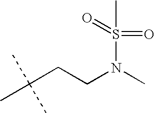 | N-(2,6- Dichlorobenzoyl)-O-{3- [methyl(methylsulfonyl) amino]ethyl}-L- tyrosine | 487 [M + H]⁺ | 2.80-2.90 (1 H, m), 2.88 (3 H, s), 2.94 (3 H, s), 3.05-3.10 (1 H, m), 3.48 (2 H, t), 4.11 (2 H, t), 4.61-4.65 (1 H, m), 6.88 (2 H, d), 7.21- 7.23 (2 H, d), 7.39- 7.46 (1 H, m), 7.38- 7.46 (2 H, m), 9.03 (1 H, d) |

### Example 38

### Methyl N-(2,6-dichlorobenzoyl)-O-(1-phenylpiperidin-4-yl)-L-tyrosinate

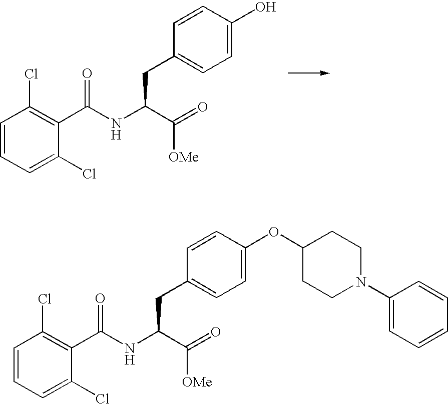

Di-tert-butyl azodicarboxylate (239 mg) was added to a solution of methyl N-(2,6-dichlorobenzoyl)-L-tyrosinate (250 mg), triphenylphosphine (268 mg) and 4-hydroxy-1-phenylpiperidine (180 mg) in DCM (15 ml) at 0° C. and the resulting reaction mixture was stirred overnight, then concentrated in vacuo. The residue was purified by chromatography using iso-hexane to 3:1 iso-hexane-ethyl acetate as eluent to give the title compound as a white foam (204 mg, 57%).

¹H NMR Spectrum (DMSO-d6) 1.70 (2H, m), 2.02 (2H, m), 2.88 (1H, dd), 3.04 (3H, m), 3.50 (2H, m), 3.66 (3H, s), 4.53 (1H, m), 4.71 (1H, m), 6.78 (1H, t), 6.92 (2H, dd), 6.97 (2H, dd), 7.21 (4H, m), 7.43 (3H, m), 9.13 (1H, d).

Mass Spectrum [M+H]⁺=527

### Example 39

### N-(2,6-Dichlorobenzoyl)-O-(1-phenylpiperidin-4-yl)-L-tyrosine

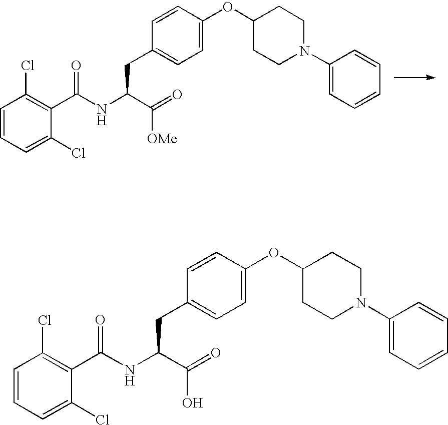

Lithium hydroxide (49 mg) in water (0.5 ml) was added to methyl N-(2,6-dichlorobenzoyl)-O-(1-phenylpiperidin-4-yl)-L-tyrosinate (204 mg) in acetonitrile (5 ml) and methanol (5 ml). The resulting reaction mixture was stirred at room temperature overnight, then concentrated in vacuo and the residue dissolved in water (2 ml). The pH was adjusted to 5 by addition of 10% aq. HCl resulting in a solid precipitate that was filtered and dried to give the title compound as a white solid (176 mg, 89%).

¹H NMR Spectrum (DMSO-d6) 1.69 (2H, m), 2.00 (2H, m), 2.90 (1H, dd), 3.02 (3H, m), 3.48 (2H, m), 4.48 (1H, m), 4.55 (1H, m), 6.75 (1H, m), 6.84 (2H, d), 6.95 (2H, m), 7.16 (4H, m), 7.34 (3H, m), 8.98 (1H, d).

Mass Spectrum [M+H]⁺=513

### Example 40

### Methyl N-(tert-butoxycarbonyl)-O-(1-phenylpiperidin-4-yl)-L-tyrosinate

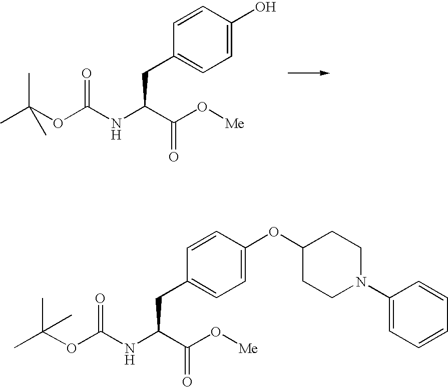

Methyl N-(tert-butoxycarbonyl)-L-tyrosinate (1.05 g), triphenylphosphine (1.40 g) and 4-hydroxy-1-phenylpiperidine (0.95 g) were dissolved in DCM and cooled to 0° C. DTAD (1.25 g) was added slowly maintaining T<5° C. The reaction mixture was stirred overnight at room temperature then concentrated in vacuo and the residue purified by chromatography using iso-hexane-10% ethyl acetate to 50% ethyl acetate as eluent to give the title compound as a yellow solid (1.91 g, 79%).

¹H NMR Spectrum (DMSO-d6) 1.33 (9H, s), 1.70 (2H, m), 2.04 (1H, m), 2.84 (2H, m), 3.04 (2H, t), 3.50 (2H, m), 3.61 (3H, s), 4.12 (1H, m), 4.51 (1H, t), 6.76 (1H, t), 6.93 (4H, dd), 7.18 (5H, m).

Mass Spectrum [M+H]⁺=455

### Example 41

### Methyl O-(1-phenylpiperidin-4-yl)-L-tyrosinate

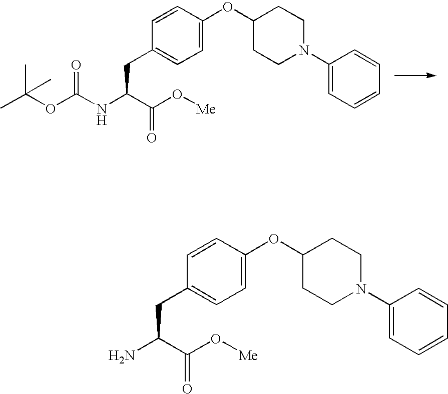

c.HCl (0.26 ml) was added to methyl N-(tert-butoxycarbonyl)-O-(1-phenylpiperidin-4-yl)-L-tyrosinate (1.91 g) in methanol (30 ml) and the solution heated at 65° C. overnight. The solution was concentrated in vacuo and the residue dissolved in methanol (5 ml) and ethyl acetate (50 ml) and washed with saturated aqueous sodium carbonate solution. The organic solvent was dried and concentrated to give the title compound as a brown oil that was used without further purification (0.93 g, 63%).

¹H NMR Spectrum (DMSO-d6) 1.70 (4H, m), 2.76 (2H, m), 3.03 (2H, ddd), 3.51 (3H, m), 3.58 (3H, s), 4.50 (1H, quintet), 6.76 (1H, t), 6.89 (2H, d), 6.96 (2H, d), 7.08 (2H, d), 7.21 (2H, dd).

Mass Spectrum [M+H]⁺=355.

### Example 42

### N-(2,6-Dimethylbenzoyl)-O-(1-phenylpiperidin-4-yl)-L-tyrosine

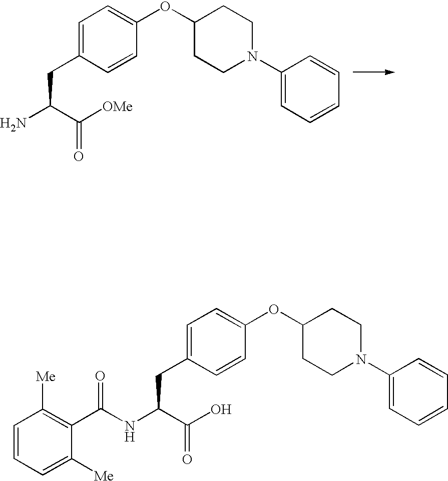

HATU (113 mg) was added to a solution of methyl O-(1-phenylpiperidin-4-yl)-L-tyrosinate (88 mg), 2,6-dimethylbenzoic acid (45 mg) and triethylamine (42 μl) in DMF (2 ml) and the resulting reaction mixture was stirred overnight. The reaction mixture was concentrated in vacuo and the residue partitioned between ethyl acetate and water, dried and concentrated. The residue was dissolved in acetonitrile (2 ml) and a solution of lithium hydroxide (31 mg) in water (0.2 ml) was added. The mixture was allowed to stir at room temperature overnight then concentrated in vacuo and the residue purified by reverse phase chromatography to give the title compound as a solid (45 mg, 38%).

¹H NMR Spectrum (DMSO-d6, 400 MHz) δ1.71 (2H, t), 2.00 (8H, m), 2.79-2.85 (1H, m), 3.03-3.09 (2H, m), 3.12-3.16 (1H, m), 3.50-3.51 (2H, m), 4.52 (1H, q), 4.61-4.64 (1H, m), 6.77 (1H, t), 6.91 (2H, d), 6.96 (4H, m), 7.11 (1H, t), 7.20-7.24 (4H, m), 8.38 (1H, d).

Mass Spectrum [M+H]⁺=473

The reaction described above was repeating using the appropriate carboxylic acid. Thus were obtained the examples described below:

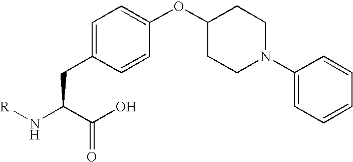

| | | | Mass | | NMR Data |
|---|---|---|---|---|---|
| Example | R | Name | Ion | RT | (400 MHz, DMSO-d₆) |
| 43 | 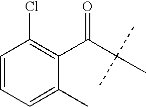 | N-(2-Chloro-6- methylbenzoyl)-O-(1- phenylpiperidin- 4-yl)-L-tyrosine | 493 [M + H]⁺ | 2.53 | 1.69-1.73 (2 H, m), 2.00-2.05 (2 H, m), 2.83-2.89 (1 H, m), 3.02-3.05 (1 H, m), 3.06-3.12 (2 H, m), 3.50 (2 H, d), 4.51- 4.54 (2 H, m), 6.77 (1 H, t), 6.89 (2 H, d), 6.96-6.98 (2 H, m), 7.07-7.08 (1 H, m), 7.13-7.17 (1 H, m), 7.20-7.26 (5 H, m), 8.59 (1 H, d) |
| 44 | 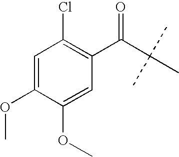 | N-(2-Chloro-4,5- dimethoxybenzoyl)-O-(1- phenylpiperidin- 4-yl)-L-tyrosine | 539 [M + H]⁺ | 2.51 | |

### Example 45

### N-(2,6-dichlorobenzoyl)-O-[1-(4-fluoro-3-methylbenzoyl)piperidin-4-yl]-L-tyrosine

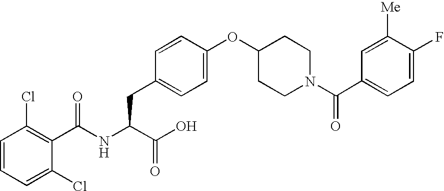

To a solution of methyl N-(2,6-dichlorobenzoyl)-O-piperidin-4-yl-L-tyrosinate hydrochloride salt (100 mgs, 0.21 mmol, 1 equiv.) in acetonitrile was added at room temperature NMM (68 μL, 0.62 mmol, 3 equivalents), then 4-fluoro-3-methylbenzoyl chloride (40 mg, 0.23 mmol, 1.1 equivalents). The mixture was stirred overnight. Water (0.3 mL) was then added. The methyl ester was saponified by treatment with LiOH.H₂O (34 mg, 0.82 mmol, 4 equivalents) at room temperature for 3 hours. The reaction mixtures were filtered, neutralized with 300 μL TFA, and purified by preparative HPLC on reversed phase silica using a solvent gradient of 10% to 100% acetonitrile in water (containing 1% acetic acid) as eluent to afford the final compound as a white solid (66 mgs, 55% yield).

Examples 46 to 53 were made using the same procedure, with the appropriate starting methyl ester precursors and acid chlorides.

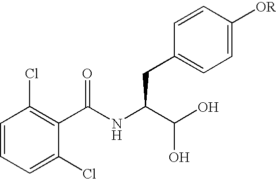

| | | Mass | NMR Data |
|---|---|---|---|
| Example | R/Name | Ion | (500 MHz, DMSO-d6) |
| 46 | 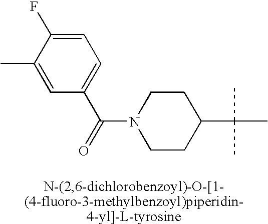 | 573 [M + H]⁺ | 1.60 (2 H, bs), 1.93 (2 H, bs), 2.26 (3 H, s), 2.93 (1 H, dd), 3.06 (1 H, dd), 3.25 (2 H, bs), 3.50 (1 H, bs), 3.93 (1 H, bs), 4.29-4.38 (1 H, m), 4.54-4.61 (1 H, m), 6.83 (2 H, d), 7.17 (2 H, d), 7.20 (1 H, d), 7.28 (1 H, ddd), 7.34-7.40 (2 H, m), 7.40-7.45 (2 H, m), 8.23 (1 H, bs) |
| 47 | 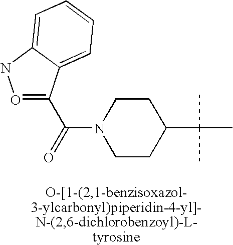 | 580 [M − H]⁻ | 1.67-1.77 (2 H, m), 1.98-2.10 (2 H, m), 2.95 (1 H, dd), 3.07 (1 H, dd), 3.59-3.70 (2 H, m), 3.88-4.04 (2 H, m), 4.28-4.35 (1 H, m), 4.63-4.69 (1 H, m), 6.86 (2 H, d), 7.18 (2 H, d), 7.25 (1 H, dd), 7.37 (1 H, dd), 7.41-7.45 (2 H, m), 7.44 (1 H, dd), 7.75 (1 H, d), 7.82 (1 H, d), 8.20 (1 H, bs) |
| 48 | 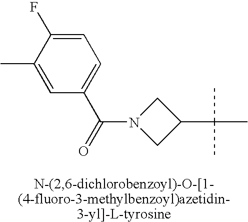 | 545 [M + H]⁺ | 2.27 (3 H, s), 2.92 (1 H, dd), 3.07 (1 H, dd), 3.90-3.99 (1 H, m), 4.23- 4.31 (1 H, m), 4.37-4.44 (1 H, m), 4.46-4.56 (1 H, m), 4.65-4.74 (1 H, m), 4.99-5.06 (1 H, m), 6.72 (2 H, d), 7.17-7.24 (3 H, m), 7.37 (1 H, dd), 7.40-7.45 (2 H, m), 7.53 (1 H, ddd), 7.61 (1 H, dd), 8.40 (1 H, bs) |
| 49 | 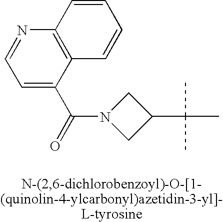 | 564 [M + H]⁺ | 2.89 (1 H, dd), 3.06 (1 H, dd), 3.87- 3.93 (1 H, m), 4.07-4.14 (1 H, m), 4.31-4.36 (1 H, m), 4.36-4.43 (1 H, m), 4.66 (1 H, dd), 5.00-5.08 (1 H, m), 6.70 (2 H, dd), 7.17 (2 H, dd), 7.35-7.43 (3 H, m), 7.61 (1 H, d), 7.71 (1 H, dd), 7.84 (1 H, ddd), 8.06-8.12 (2 H, m), 8.39 (1 H, bs), 8.97 (1 H, dd) |
| 50 | 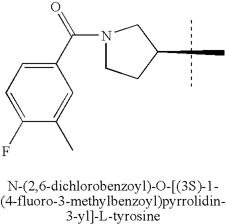 | Not available | (mixture of 2 rotamers 50/50) 1.98-2.29 (2 H, m), 2.23 (1.5 H, s), 2.28 (1.5 H, s), 2.76-2.94 (1 H, m), 2.99-3.11 (1 H, m), 3.49-3.55 (0.5 H, m), 3.56-3.68 (2.5 H, m), 3.77-3.90 (1 H, m), 4.54-4.68 (1 H, m), 4.98 (0.5 H, bs), 5.07 (0.5 H, bs), 6.81 (1 H, d), 6.89 (1 H, d), 7.13-7.27 (3 H, m), 7.35-7.49 (4.5 H, m), 7.51 (0.5 H, d), 9.00 (0.5 H, d), 9.05 (0.5 H, d), 12.74 (1 H, bs) |
| 51 | 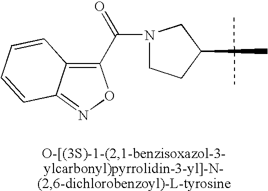 | 568 [M + H]⁺ | (mixture of 2 rotamers 50/50) 2.12-2.36 (2 H, m), 2.78-2.91 (1 H, m), 3.03-3.01 (1 H, m), 3.65-3.75 (0.5 H, m), 3.77-3.85 (0.5 H, m), 3.85-3.92 (1 H, m), 4.01-4.09 (1 H, m), 4.22 (0.5 H, ddd), 4.31 (0.5 H, dd), 4.58-4.57 (1 H, m), 5.12 (0.5 H, bs), 5.19 (0.5 H, bs), 6.89 (1 H, d), 6.92 (1 H, d), 7.21 (1 H, d), 7.24 (1 H, d), 7.25-7.31 (1 H, m), 7.34-7.46 (3 H, m), 7.47-7.54 (1 H, m), 7.77 (0.5 H, d), 7.78 (0.5 H, d), 7.96 (0.5 H, s), 7.97 (0.5 H, s), 8.98 (0.5 H, d), 9.03 (0.5 H, d), 12.74 (1 H, bs) |
| 52 | 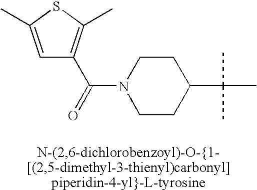 | Not available | 1.55 (2 H, bs), 1.90 (2 H, bs), 2.31 (3 H, s), 2.37 (3 H, s), 2.85 (1 H, dd), 3.06 (1 H, dd), 3.26 (2 H, bs), 3.51 (1 H, bs), 3.91 (1 H, bs), 4.55- 4.65 (2 H, m), 6.64 (1 H, d), 6.88 (2 H, d), 7.19 (2 H, d), 7.38 (1 H, dd), 7.41-7.46 (2 H, m), 8.98 (1 H, bs), 12.75 (1 H, bs) |
| 53 | 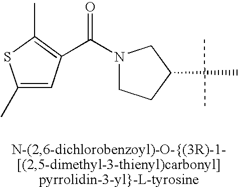 | Not available | (mixture of 2 rotamers 50/50) 1.99-2.25 (2 H, m), 2.32 (3 H, s), 2.36 (1.5 H, s), 2.37 (1.5 H, s), 2.79-2.92 (1 H, m), 3.02-3.11 (1 H, m), 3.35-3.50 (1.5 H, m), 3.52- 3.62 (1.5 H, m), 3.66 (0.5 H, dd), 3.74 (0.5 H, dd), 4.61 (1 H, bs), 4.97 (0.5 H, bs), 5.04 (0.5 H, bs), 6.67 (0.5 H, s), 6.75 (0.5 H, s), 6.82 (1 H, d), 6.88 (1 H, d), 7.18 (1 H, d), 7.22 (1 H, d), 7.36-7.46 (3 H, m), 8.99 (1 H, bs), 12.75 (1 H, bs) |

### Example 54

### N-(2,6-dichlorobenzoyl)-O-[1-(4-fluorobenzyl)azetidin-3-yl]-L-tyrosine

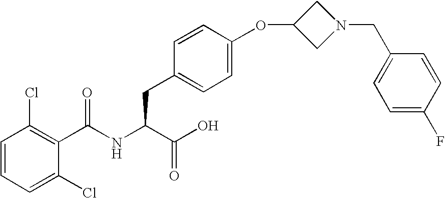

To a solution of O-azetidin-3-yl-N-(2,6-dichlorobenzoyl)-L-tyrosine hydrochloride salt 11 (100 mgs, 0.22 mmol, 1 equivalent) in acetonitrile was added at room temperature DIPEA (115 μL, 0.66 mmol, 3 equivalents), then 4-fluorobenzyl bromide (25 μL, 0.20 mmol, 0.9 equivalent). The mixture was stirred overnight. Water (0.3 mL) was added, and the methyl ester was saponified by treatment with LiOH.H₂O (34 mg, 0.82 mmol, 4 equivalents) at room temperature for 3 hours. The reaction mixtures were filtered, neutralized with 300 μL TFA and purified by preparative HPLC on reversed phase silica using a solvent gradient of 10% to 100% acetonitrile in water (containing 2 g/L of (NH₄)₂CO₃) as eluent to afford the final compound 54 as a white solid (71 mgs, 65% yield).

Examples 55 to 61 were made using the same procedure, with the appropriate benzyl chlorides or bromides.

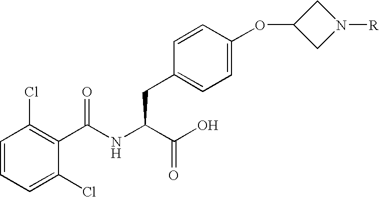

| | | Mass | NMR Data |
|---|---|---|---|
| Example | R/Name | Ion | (500 MHz, DMSO-d6) |
| 55 | 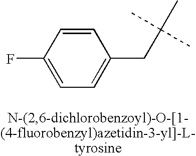 | 517 [M + H]⁺ | 2.90 (1 H, dd), 2.97-3.02 (2 H, m), 3.05 (1 H, dd), 3.60 (2 H, s), 3.66-3.72 (2 H, m), 4.36- 4.45 (1 H, m), 4.69-4.78 (1 H, m), 6.68 (2 H, d), 7.09-7.18 (4 H, m), 7.32 (2 H, dd), 7.37 (1 H, dd), 7.40-7.45 (2 H, m), 8.47 (1 H, m) |
| 56 | 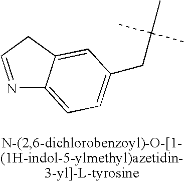 | 538 [M + H]⁺ | 2.82 (1 H, dd), 2.99-3.09 (3 H, m), 3.72 (4 H, bs), 4.54-4.63 (1 H, m), 4.71-4.79 (1 H, m), 6.37 (1 H, dd), 6.72 (2 H, d), 7.02 (1 H, dd), 7.17 (2 H, d), 7.29-7.35 (2 H, m), 7.37 (1 H, dd), 7.39-7.47 (3 H, m), 8.97 (1 H, d), 11.02 (1 H, s) |
| 57 | 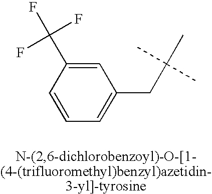 | 567 [M + H]⁺ | 2.91 (1 H, dd), 3.01-3.09 (m, 3 H), 3.70-3.76 (4 H, m), 4.34 (1 H, bs), 4.73-4.80 (1 H, m), 6.68 (2 H, d), 7.15 (2 H, d), 7.37 (1 H, dd), 7.40-7.44 (2 H, m), 7.53-7.58 (1 H, m), 7.59- 7.65 (3 H, m), 8.30 (1 H, bs) |
| 58 | 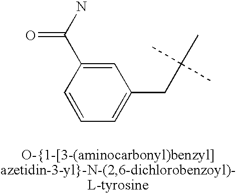 | 542 [M + H]⁺ | 2.87 (1 H, dd), 2.99-3.09 (3 H, m), 3.68 (2 H, d), 3.72 (2 H, dd), 4.44-4.53 (1 H, m), 4.72- 4.79 (1 H, m), 6.71 (2 H, d), 7.17 (2 H, d), 7.32 (1 H, s), 7.35-7.46 (5 H, m), 7.74 (1 H, d), 7.75 (1 H, s), 7.96 (1 H, s), 8.65 (1 H, bs) |
| 59 | 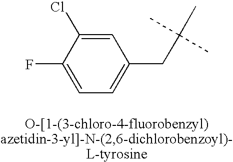 | 551 [M + H]⁺ | 2.84 (1 H, dd), 3.00-3.11 (3 H, m), 3.64 (2 H, s), 3.69-3.78 (2 H, m), 4.57-4.66 (1 H, m), 7.73-4.81 (1 H, m), 6.73 (2 H, d), 7.18 (2 H, d), 7.32 (1 H, dd), 7.37 (1 H, dd), 7.39-7.44 (3 H, m), 7.50 (1 H, dd), 8.02 (1 H, d) |
| 60 | 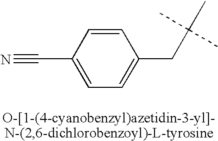 | 524 [M + H]⁺ | 2.84 (dd, 1 H), 3.02-3.10 (3 H, m), 370-3.78 (4 H, m), 4.55- 4.64 (1 H, m), 4.75-4.82 (1 H, m), 6.73 (2 H, d), 7.18 (2 H, d), 7.38 (1 H, dd), 7.41-7.45 (2 H, m), 7.49 (2 H, d), 7.79 (2 H, d), 8.97 (1 H, d) |
| 61 | 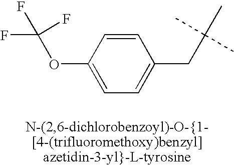 | 583 [M + H]⁺ | 2.84 (1 H, dd), 3.04-3.12 (2 H, m), 3.05 (1 H, dd), 3.70 (2 H, bs), 3.76 (2 H, bs), 4.61 (1 H, ddd), 4.74-4.81 (1 H, m), 6.73 (2 H, d), 7.18 (2 H, d), 7.31 (2 H, d), 7.38 (1 H, dd), 7.40- 7.45 (4 H, m), 8.02 (1 H, d) |

### Example 62

### O-[(3S)-1-benzylpyrrolidin-3-yl]-N-(2,6-dichlorobenzoyl)-L-tyrosine

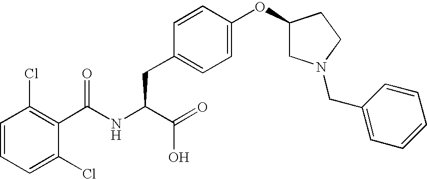

To a solution of N-(2,6-dichlorobenzoyl)-O-[(3S)-pyrrolidin-3-yl]-L-tyrosine hydrochloride salt 13 (100 mgs, 0.21 mmol, 1 equivalent) in acetonitrile was added at 4° C. DIPEA (110 μL, 0.63 mmol, 3 equivalents), then benzyl bromide (32 mg, 0.19 mmol, 0.9 equivalents). The mixture was stirred overnight at 4° C. Then 0.3 mL water were added, and the methyl ester was saponified by treatment with LiOH.H₂O (37 mg, 0.87 mmol, 4 equiv.) at room temperature for 3 hours. The reaction mixtures were filtered, neutralized with 300 μL TFA and purified by preparative HPLC on reversed phase silica using a solvent gradient of 10% to 100% acetonitrile in water (containing 0.1% TFA) as eluent. Subsequent dissolution of the resulting TFA salts into a solution of MeOH/NH₃ (7N) afforded after evaporation the final compound 62 as a white solid (83 mgs, 77% yield).

(Numbers Changed Made in the Text and in the Tables)

Examples 63 to 69 were made using the same procedure, with the appropriate benzyl chlorides or bromides, and from intermediate 13 or its enantiomer 10. With benzyl chlorides, the reaction might need to be warmed up to room temperature to reach completion.

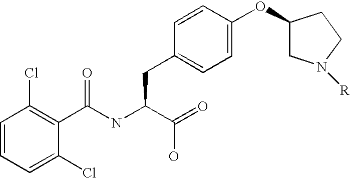

| | | Mass | NMR Data |
|---|---|---|---|
| Example | R/Name | Ion | (500 MHz, DMSO-d6) |
| 62 | 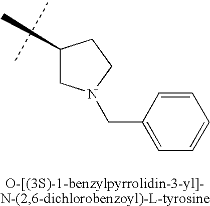 | 513. [M + H]⁺ | 1.71-1.80 (1 H, m), 2.21- 2.31 (1 H, m), 2.41-2.47 (1 H, m), 2.56 (1 H, dd), 2.65-2.72 (1 H, m), 2.80- 2.89 (2 H, m), 3.05 (1 H, dd), 3.57 (1 H, d), 3.61 (1 H, d), 4.52-4.60 (1 H, m), 4.79- 4.86 (1 H, m), 6.75 (2 H, d), 7.16 (2 H, d), 7.21-7.27 (1 H, m), 7.29-7.33 (3 H, m), 7.38-7.43 (3 H, m), 8.88 (1 H, d) |
| 63 | 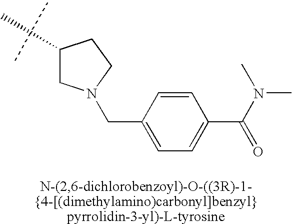 | 584 [M + H]⁺ | 1.79 (1 H, bs), 2.23-2.36 (1 H, m), 2.57-3.07 (4 H, m), 2.83 (1 H, dd), 2.89 (3 H, bs), 2.96 (3 H, bs), 3.05 (1 H, dd), 3.70 (2 H, bs), 4.57-4.67 (1 H, m), 4.87 (1 H, bs), 6.78 (2 H, d), 7.17 (2 H, d), 7.30-7.48 (7 H, m), 9.02 (1 H, d) |
| 64 | 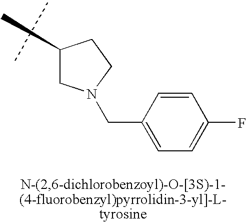 | 531 [M + H]⁺ | 1.70-1.80 (1 H, m), 2.20- 2.30 (1 H, m), 2.39-2.46 (1 H, m), 2.55 (1 H, dd), 2.64-2.70 (1 H, m), 2.82 (1 H, dd), 2.88 (1 H, dd), 3.04 (1 H, dd), 3.55 (1 H, d), 3.59 (1 H, d), 4.39-4.47 (1 H, m), 4.79-4.84 (1 H, m), 6.72 (2 H, d), 7.09-7.17 (4 H, m), 7.31-7.36 (2 H, m), 7.37-7.43 (3 H, m), 8.52 (1 H, bs) |
| 65 | 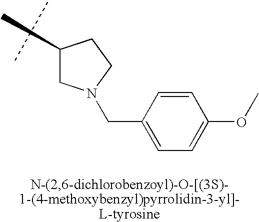 | 543 [M + H]⁺ | 1.69-1.79 (1 H, m), 2.20- 2.30 (1 H, m), 2.38-2.45 (1 H, m), 2.53 (1 H, dd), 2.62-2.70 (1 H, m), 2.80 (1 H, dd), 2.84 (1 H, dd), 3.04 (1 H, dd), 3.50 (1 H, d), 3.55 (1 H, d), 3.72 (3 H, s), 3.51-3.59 (1 H, m), 4.78- 4.85 (1 H, m), 6.74 (2 H, d), 6.87 (2 H, d), 7.15 (2 H, d), 7.22 (2 H, d), 7.35-7.44 (3 H, m), 8.86 (1 H, d) |
| 66 | 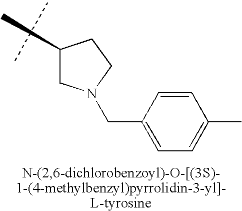 | 527 [M + H]⁺ | 1.69-1.79 (1 H, m), 2.17- 2.28 (1 H, m), 2.27 (3 H, s), 2.38-2.46 (1 H, m), 2.54 (1 H, dd), 2.62-2.70 (1 H, m), 2.80 (1 H, dd), 2.86 (1 H, dd), 3.05 (1 H, dd), 3.52 (1 H, d), 3.56 (1 H, d), 4.45-4.52 (1 H, m), 4.77- 4.84 (1 H, m), 6.73 (2 H, d), 7.11 (2 H, d), 7.15 (2 H, d), 7.18 (2 H, d), 7.35-7.44 (3 H, m), 8.61 (1 H, d) |
| 67 | 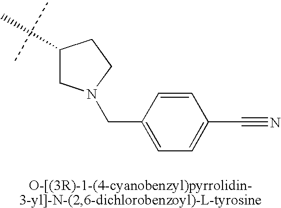 | 538 [M + H]⁺ | 1.72-1.83 (1 H, m), 2.21- 2.31 (1 H, m), 2.41-2.50 (1 H, m), 2.59 (1 H, dd), 2.66-2.74 (1 H, m), 2.84 (1 H, dd), 2.91 (1 H, dd), 3.05 (1 H, dd), 3.67 (1 H, d), 3.71 (1 H, d), 4.29-4.38 (1 H, m), 4.79-4.86 (1 H, m), 6.71 (2 H, d), 7.14 (2 H, d), 7.37 (1 H, dd), 7.39-7.44 (2 H, m), 7.52 (2 H, d), 7.78 (2 H, d), 8.24 (1 H, bs) |
| 68 | 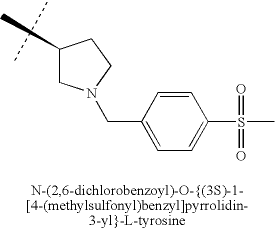 | 591 [M + H]⁺ | 1.82 (1 H, bs), 2.29 (1 H, bS), 2.40-3.14 (4 H, m), 2.83 (1 H, dd), 3.05 (1 H, dd), 3.20 (3 H, s), 3.75 (2 H, bs), 4.57-4.65 (1 H, m), 4.88 (1 H, bs), 6.78 (2 H, d), 7.18 (2 H, d), 7.36-7.45 (3 H, m), 7.62 (2 H, bs), 7.90 (2 H, d), 9.02 (1 H, d) |
| 69 | 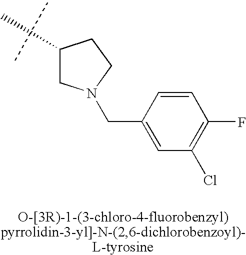 | 565 [M + H]⁺ | 1.72-1.81 (1 H, m), 2.21- 2.32 (1 H, m), 2.41-2.47 (1 H, m), 2.57 (1 H, dd), 2.65-2.72 (1 H, m), 2.84 (1 H, dd), 2.87 (1 H, dd), 3.05 (1 H, dd), 3.59 (2 H, s), 4.42-4.52 (1 H, m), 4.79- 4.86 (1 H, m), 6.74 (2 H, d), 7.16 (2 H, d), 7.30-7.45 (5 H, m), 7.51 (1 H, dd), 8.63 (1 H, bs) |

### Example 70

### N-(2,6-dichlorobenzoyl)-O-[(3R)-1-(4-methylbenzyl)piperidin-3-yl]-L-tyrosine

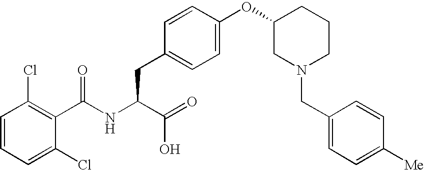

To a solution of N-(2,6-dichlorobenzoyl)-O-[(3R)-piperidin-3-yl]-L-tyrosine hydrochloride salt 14 (102 mgs, 0.21 mmol, 1 equivalent) in acetonitrile was added at 4° C. DIPEA (110 μL, 0.63 mmol, 3 equivalents), then 4-methylbenzyl bromide (35 mg, 0.19 mmol, 0.9 equivalents). The mixture was stirred overnight at 4° C. Then 0.3 mL water were added, and the methyl ester was saponified by treatment with LiOH.H₂O (37 mg, 0.87 mmol, 4 equivalents) at room temperature for 3 hours. The reaction mixtures were filtered, neutralized with 300 μL TFA and purified by preparative HPLC on reversed phase silica using a solvent gradient of 10% to 100% acetonitrile in water (containing 0.1% TFA) as eluent. Subsequent dissolution of the resulting TFA salts into a solution of MeOH/NH₃ (7N) afforded after evaporation the final compound 70 as a white solid (95 mgs, 84% yield).

Examples 71 to 77 were made using the same procedure, with the appropriate benzyl chlorides or bromides, and from intermediate 14 or its enantiomer 15. With benzyl chlorides, the reaction might need to be warmed up to room temperature to reach completion.

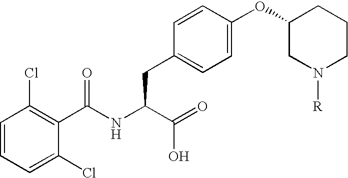

| | | Mass | NMR Data |
|---|---|---|---|
| Example | R/Name | Ion | (500 MHz, DMSO-d6) |
| 70 | 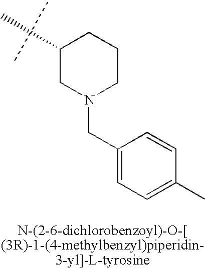 | 541 [M + H]⁺ | 1.22-1.33 (1 H, m), 1.46-1.57 (1 H, m), 1.64-1.73 (1 H, m), 1.93-2.08 (3 H, m), 2.26 (3 H, s), 2.57-2.64 (1 H, m), 2.86 (1 H, dd), 2.88-2.94 (1 H, m), 3.04 (1 H, dd), 3.46 (2 H, s), 4.25-4.33 (1 H, m), 4.44-4.53 (1 H, m), 6.79 (2 H, d), 7.11 (2 H, d), 7.15 (2 H, d), 7.17 (2 H, d 7.36-7.44 (3 H, m), 8.68 (1 H, bs) |
| 71 | 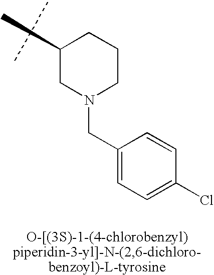 | 561 [M + H]⁺ | 1.25-1.36 (1 H, m), 1.46-1.58 (1 H, m), 1.66-1.73 (1 H, m), 1.93-2.12 (3 H, m), 2.55-2.63 (1 H, m), 2.86-2.94 (1 H, m), 2.87 (1 H, dd), 3.05 (1 H, dd), 3.50 (3 H, s), 4.26-4.34 (1 H, m), 4.42- 4.50 (1 H, m), 6.78 (1 H, d), 7.15 (2 H, d), 7.32 (2 H, d), 7.34-7.40 (3 H, m), 7.40-7.45 (m, 2 H), 8.54 (1 H, bs) |
| 72 | 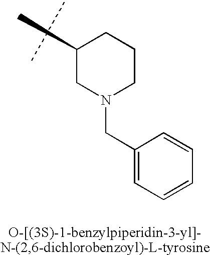 | 527 [M + H]⁺ | 1.21-1.35 (1 H, m), 1.47-1.59 (1 H, m), 1.65-1.74 (1 H, m), 1.93-2.09 (3 H, m), 2.58-2.66 (1 H, m), 2.86 (1 H, dd), 2.90- 2.96 (1 H, m), 3.04 (1 H, dd), 3.51 (2 H, s), 4.26-4.35 (1 H, m), 4.45-4.53 (1 H, m), 6.78 (2 H, d), 7.15 (2 H, d), 7.20-7.26 (1 H, m), 7.27-7.34 (4 H, m), 7.38 (1 H, dd), 7.40-7.45 (2 H, m), 8.68 (1 H, bs) |
| 73 | 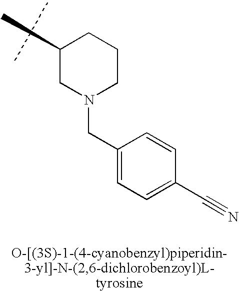 | 552 [M + H]⁺ | 1.28-1.39 (1 H, m), 1.49-1.60 (1 H, m), 1.67-1.76 (1 H, m), 1.93-2.02 (2 H, m), 2.04-2.17 (1 H, m), 2.56-2.64 (1 H, m), 2.85 (1 H, dd), 2.85-2.94 (1 H, m), 3.05 (1 H, dd), 3.61 (2 H, s), 4.29-4.37 (1 H, m), 4.48-4.57 (1 H, m), 6.80 (2 H, d), 7.15 (2 H, d), 7.35-7.44 (3 H, m), 7.51 (2 H, d), 7.78 (2 H, d), 8.77 (1 H, bs) |
| 74 | 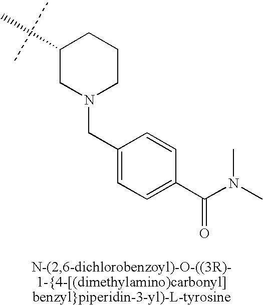 | 598 [M + H]⁺ | 1.35 (1 H, bs), 1.57 (1 H, bs), 1.74 (1 H, bs), 2.00 (1 H, bs), 2.09 (2 H, bs), 2.65 (1 H, bs), 2.83 (1 H, dd), 2.88 (3 H, bs), 2.94 (3 H, bs), 2.97 (1 H, bs), 3.05 (1 H, dd), 3.56 (2 H, bs), 4.35 (1 H, bs), 4.57-4.65 (1 H, m), 6.83 (2 H, bs), 7.17 (2 H, d), 7.29-7.49 (7 H, m), 9.03 (1 H, d) |
| 75 | 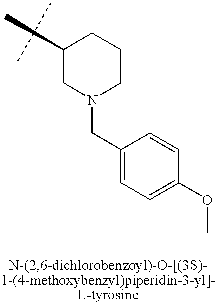 | 557 [M + H]⁺ | 1.22-1.33 (1 H, m), 1.45-1.56 (1 H, m), 1.63-1.73 (1 H, m), 1.93-2.07 (3 H, m), 2.57-2.64 (1 H, m), 2.87 (1 H, dd), 2.88- 2.95 (1 H, m), 3.04 (1 H, m), 3.43 (2 H, s), 3.72 (3 H, s), 4.24-4.33 (1 H, m), 4.42-4.52 (1 H, m), 6.78 (2 H, d), 6.86 (2 H, d), 7.14 (2 H, d), 7.37 (1 H, dd), 7.40-7.45 (2 H, m), 8.62 (1 H, bs) |
| 76 | 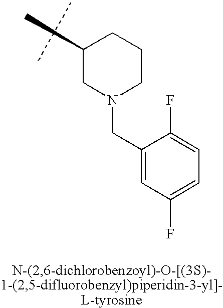 | 563 [M + H]⁺ | 1.25-1.36 (1 H, m), 1.49-1.59 (1 H, m), 1.67-1.75 (1 H, m), 1.92-2.00 (1 H, m), 2.06-2.18 (2 H, m), 2.59-2.68 (1 H, m), 2.84 (1 H, dd), 2.88-2.96 (1 H, m), 3.05 (1 H, dd), 3.58 (2 H, s), 4.30-4.39 (1 H, m), 4.60 (1 H, ddd), 6.83 (2 H, d), 7.11-7.18 (1 H, m), 7.17 (2 H, d), 7.19-7.28 (2 H, m), 7.36-7.45 (3 H, m), 8.99 (1 H, d) |
| 77 | 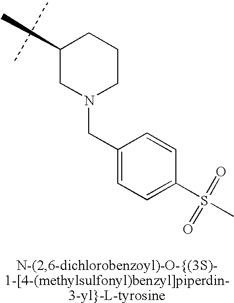 | 605 [M + H]⁺ | 1.34 (1 H, bs), 1.56 (1 H, bs), 1.72 (1 H, bs), 1.99 (1 H, bs), 2.03-2.23 (2 H, m), 2.55-2.66 (1 H, bs), 2.83 (1 H, dd), 2.92 (1 H, bs), 3.05 (1 H, dd), 3.19 (3 H, s), 3.63 (2 H, bs), 4.36 (1 H, bs), 4.61 (1 H, ddd), 6.84 (2 H, d), 7.17 (2 H, d), 7.36-7.45 (3 H, m), 7.59 (2 H, bs), 7.88 (2 H, bs), 9.03 (1 H, d) |

### Example 78

### N-(2,6-dichlorobenzoyl)-O-[1-(4-fluorobenzyl)piperidin-4-yl]-L-tyrosine

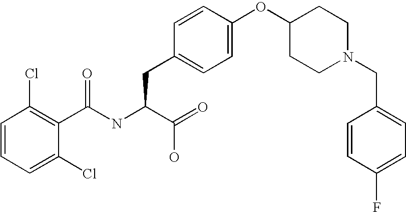

To a solution of N-(2,6-dichlorobenzoyl)-O-piperidin-4-yl-L-tyrosine hydrochloride salt 12 (100 mgs, 0.20 mmol, 1 equivalent) in acetonitrile was added at −10° C. DIPEA (106 μL, 0.61 mmol, 3 equivalents), then 4-fluorobenzyl bromide (36 mg, 0.18 mmol, 0.9 equivalents). The mixture was stirred overnight at room temperature. Then 0.3 mL water were added, and the methyl ester was saponified by treatment with LiOH.H₂O (34 mg, 0.82 mmol, 4 equivalents) at room temperature for 3 hours. The reaction mixtures were filtered, neutralized with 300 μL TFA and purified by preparative HPLC on reversed phase silica using a solvent gradient of 10% to 100% acetonitrile in water (containing 0.1% TFA) as eluent. Subsequent dissolution of the resulting TFA salts into a solution of MeOH/NH₃ (7N) afforded after evaporation the final compound 78 as a white solid (88 mgs, 81% yield).

Examples 79 to 86 were made using the same procedure, with the appropriate benzyl chlorides or bromides.

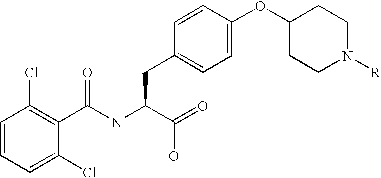

| | | Mass | NMR Data |
|---|---|---|---|
| Example | R/Name | Ion | (500 MHz, DMSO-d6) |
| 78 | 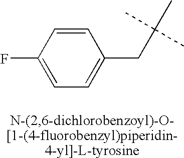 | 545 [M + H]⁺ | 1.53-1.65 (2 H, m), 1.85- 1.95 (2 H, m), 2.17-2.26 (2 H, m), 2.60-2.69 (2 H, m), 2.89 (1 H, dd), 3.50 (1 H, dd), 3.46 (2 H, s), 4.27-4.35 (1 H, m), 4.39- 4.48 (1 H, m), 6.80 (2 H, d), 7.10-7.19 (4 H, m), 7.30-7.40 (3 H, m), 7.40- 7.45 (2 H, m), 8.51 (1 H, bs)1/40 |
| 79 | 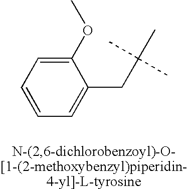 | 557 [M + H]⁺ | 1.55-1.66 (2 H, m), 1.86- 1.95 (2 H, m), 2.20-2.30 (2 H, m), 2.65-2.74 (2 H), 2.87 (1 H, dd), 3.05 (1 H, dd), 3.48 (2 H, s), 3.77 (3 H, s), 4.27-4.35 (1 H, s), 4.45-4.54, (1 H, m), 6.81 (2 H, d), 6.92 (1 H, dd), 6.97 (1 H, d), 7.46 (2 H, d), 7.22 (1 H, ddd), 7.31 (1 H, dd), 7.37 (1 H, dd), 7.41-7.45 (2 H, m), 8.70 (bs, 1 H) |
| 80 | 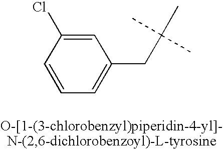 | 561 [M + H]⁺ | 1.55-1.66 (2 H, m), 1.85- 1.96 (2 H, m), 2.18-2.29 (2 H, m), 2.59-2.71 (2 H, m), 2.89 (1 H, dd), 3.50 (1 H, dd), 3.28-3.36 (2 H, s), 4.27-4.36 (1 H, m), 4.39-4.50 (1 H, m), 6.80 (2 H, d), 7.16 (2 H, d), 7.28 (1 H, ddd), 7.31 (1 H, ddd), 7.33-7.40 (3 H, m), 7.40-7.45 (2 H, m), 8.52 (1 H, bs) |
| 81 | 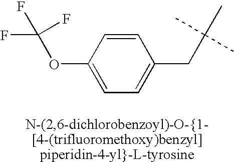 | 611 [M + H]⁺ | 1.55-1.66 (2 H, m), 1.86- 1.95 (2 H, m), 2.19-2.29 (2 H, m), 2.61-2.70 (2 H, m), 2.85 (1 H, dd), 3.05 (1 H, dd), 3.51 (2 H, s), 4.30-4.38 (1 H, m), 4.58 (1 H, ddd), 6.84 (2 H, d), 7.17 (2 H, d), 7.32 (2 H, d), 7.38 (1 H, dd); 7.41- 7.47 (4 H, m), 8.94 (1 H, d) |
| 82 | 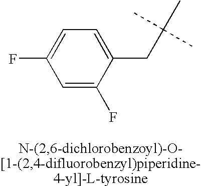 | 563 [M + H]⁺ | 1.51-1.72 (2 H, m), 1.84- 2.01 (2 H, m), 2.29 (2 H, bs), 2.71 (2 H, bs), 2.84 (1 H, dd), 3.05 (1 H, dd), 3.54 (2 H, bs), 4.36 (1 H, bs), 4.61 (1 H, ddd), 6.85 (2 H, d), 6.93-7.32 (2 H, m), 7.18 (2 H, d), 7.38 (1 H, dd), 7.41-7.55 (2 H, m), 7.49 (1 H, bs), 9.03 (1 H, d) |
| 83 | 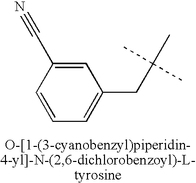 | 552 [M + H]⁺ | 1.56-1.67 (2 H, m), 1.86- 1.95 (2 H, m), 2.20-2.31 (2 H, m), 2.60-2.69 (2 H, m), 2.87 (1 H, dd), 3.05 (1 H, dd), 3.55 (2 H, s), 4.39-4.37 (1 H, m), 4.44- 4.52 (1 H, m), 6.81 (2 H, d), 7.16 (2 H, d), 7.37 (1 H, dd), 7.41-7.45 (2 H, m), 7.55 (1 H, dd), 7.67 (1 H, d), 7.73 (1 H, d), 7.75 (1 H, s), 8.73 (1 H, bs) |
| 84 | 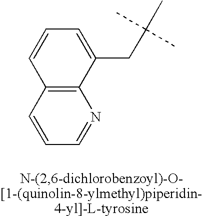 | 578 [M + H]⁺ | 1.61-1.72 (2 H, m), 1.90- 1.99 (2 H, m), 2.34-2.45 (2 H, m), 2.77-2.90 (2 H, m), 2.85 (1 H, dd), 3.05 (1 H, dd), 4.22 (2 H, s), 4.33-4.41 (m, 1 H), 4.59 (1 H, ddd), 6.85 (2 H, d), 7.17 (2 H, d), 7.38 (1 H, dd), 7.40-7.46 (2 H, m), 7.55 (1 H, dd), 7.61 (1 H, dd), 7.85 (1 H, d), 7.89 (1 H, d), 8.37 (1 H dd), 8.93 (1 H, dd), 8.95 (1 H, d) |
| 85 | 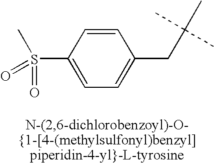 | 605 [M + H]⁺ | 1.57-1.68 (2 H, m), 1.87- 1.97 (2 H, m), 2.21-2.32 (2 H, m), 2.63-2.71 (2 H, m), 2.87 (1 H, dd), 3.05 (1 H, dd), 3.20 (3 H, s), 3.60 (2 H, d), 4.31-4.39 (1 H, m), 4.50 (1 H, bs), 6.82 (2 H, d), 7.17 (2 H, d), 7.38 (1 H, dd), 7.40- 7.45 (2 H, m), 7.59 (2 H, d), 7.88 (2 H, d), 8.76 (1 H, m) |
| 86 | 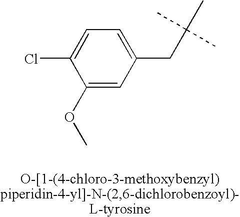 | 591 [M + H]⁺ | 1.56-1.67 (2 H, m), 1.85- 1.96 (2 H, m), 2.18-2.29 (2 H, m), 2.61-2.72 (2 H, m), 2.88 (1 H, dd), 3.05 (1 H, dd), 3.48 (2 H, s), 3.85 (3 H, s), 4.28-4.37 (1 H, m), 4.43-4.52 (1 H, m), 6.81 (2 H, d), 6.90 (1 H, dd), 7.08 (1 H, d), 7.16 (2 H, d), 7.35 (1 H, d), 7.36-7.39 (1 H, m), 7.40-7.45 (2 H, m), 8.62 (1 H, bs) |

What is claimed is:

1-15. (canceled)

16. A compound of formula I: 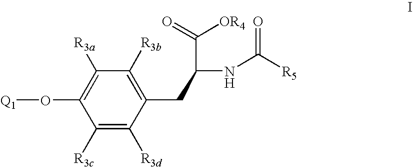
or a pharmaceutical acceptable salt thereof, wherein:
Q₁ is:
(i) a group of sub-formula (ai) 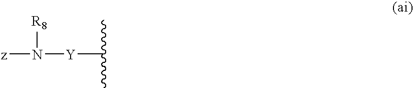
wherein Y is a (C₁-C₆)alkylene group,
Z is hydrogen or a (C₁-C₆)alkyl group,
R₈ is hydrogen, or an optionally substituted group selected from (C₁-C₆)alkyl, (C₁-C₆)alkyl sulphonyl or (C₂-C₆)alkanoyl, wherein optional substituents are one or more groups selected from halo, trifluoromethyl, cyano, nitro, hydroxy, amino, carboxy, carbamoyl, sulfamoyl, (1-6C)alkoxy, (1-6C)alkylthio, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
Or Q₁ is:
(ii) a group of the sub-formula (bi): 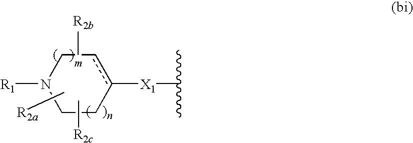
wherein:
the “- - -” is either a bond or is absent;
X₁ is a bond or (C₁-C₄)alkylene;
R₁ is
(a) H, or an optionally substituted group selected from (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aryl, heteroaryl, aralkyl, or heteroaralkyl;
(b) 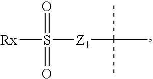
wherein [symbol] indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl;
(c) 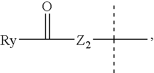
wherein [symbol] indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocyclyl, (C₃-C₆)cycloalkyl(C₁-C₆)alkylene, heterocyclyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
n and m are each independently 0, 1, or 2;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently hydrogen, halo, (C₁-C₃)alkyl, or (C₁-C₃)alkoxy;
R₄ is selected from hydrogen, (1-6C)alkyl, aryl, aryl-(1-6C)alkyl, heterocyclyl, heteroaryl, heteroaryl-(1-6C)alkyl and heterocyclyl-(1-6C)alkyl, any of which optionally bears on carbon one or more R²¹ substituents, which may be the same or different,
R₅ is aryl which is ortho-substituted with at least one group selected from (C₁-C₃)alkyl, halogen and halo-(1-3C)alkyl, and which is optionally additionally substituted with 1 or 2 groups selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
R₁₃—X₂—
wherein X₂ is a direct bond or is selected from O, S, SO, SO₂, N(R₁₄), CO, CH(OR₁₄), CON(R₁₄), N(R₁₄)CO, SO₂N(R₁₄), N(R₁₄)SO₂, OC(R₁₄)₂, SC(R₁₄)₂ and N(R₁₄)C(R₁₄)₂, wherein R₁₄ is hydrogen or (1-6C)alkyl, and R₁₃ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl; and
R²¹ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
—X⁴—R¹⁶
wherein X⁴ is a direct bond or is selected from O, CO and N(R¹⁶), wherein R¹⁶ is hydrogen or (1-6C)alkyl, and R¹⁶ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X³-Q²
wherein X³ is a direct bond or is selected from O, S, SO, SO₂, N(R¹⁷), CO, CH(OR¹⁷), CON(R¹⁷), N(R¹⁷)CO, SO₂N(R¹⁷), N(R¹⁷)SO₂, OC(R¹⁷)₂, SC(R¹⁷)₂ and N(R¹⁷)C(R¹⁷)₂, wherein R¹⁷ is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R²¹ optionally bears on carbon one or more R¹⁸,
and wherein if any heteroaryl or heterocyclyl group within R²¹ contains an —NH-moiety, the nitrogen of said moiety optionally bears a group selected from R¹⁹,
and wherein any heterocyclyl group within a substituent R²¹ optionally bears 1 or 2 oxo or thioxo substituents;
R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;
R¹⁹ is selected from carbamoyl, sulfamoyl, (1-6C)alkyl, (2-8C)alkenyl, (2-8C)alkynyl, (1-6C)alkylsulfonyl, N-(1-6C)alkylcarbamoyl, N N-di-[(1-6C)alkyl]carbamoyl, (2-6C)alkanoyl, N-(1-6C)alkylsulfamoyl and N,N-di-[(1-6C)alkyl]sulfamoyl,
or from a group of the formula:
—X⁶-Q⁴
wherein X⁶ is a direct bond or is selected from CO, SO₂, CON(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl.

17. A compound according to claim 16 wherein R₅ is a group of sub-formula (iii) 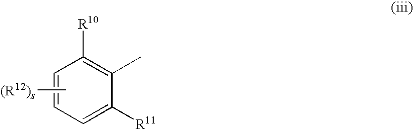
wherein:
R¹⁰ is selected from halo, (1-3C)alkyl and halo-(1-3C)alkyl;
s is 0, 1, 2 or 3
each R¹², which may be the same or different, is selected from
halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
R₁₃—X₂—
wherein R₁₃ and X₂ are as defined in relation to formula (I), and
R₁₁ is selected from hydrogen or a group as listed above for R₁₂.

18. A compound according to claim 17 wherein R₁₀ is chloro.

19. A compound according to claim 16 wherein Q₁ is a group of sub-formula (ai) in which Y is a C₁₋₃alkylene group and Z is hydrogen or a C₁₋₃ alkyl group.

20. A compound according to claim 19 wherein R₈ is hydrogen, or unsubstituted (C₁-C₆)alkyl or unsubstituted (C₁-C₆)alkylsulphonyl or acetyl.

21. A compound according to claim 16 wherein Q₁ is a group of sub-formula (bi).

22. A compound according to claim 21 wherein X₁ is a bond or a methylene group.

23. A compound according to claim 21 wherein the “- - -” is absent.

24. A compound according to claim 19 wherein the sum of m+n is equal to 0, 1, 2 or 3.

25. A compound which is
N-(2,6-Dichlorobenzoyl)-O-piperidin-4-yl-L-tyrosine;
N-(2,6-Dichlorobenzoyl)-O-[(3R)-pyrrolidin-3-yl]-L-tyrosine;
N-(2,6-Dichlorobenzoyl)-O-[(2S)-pyrrolidin-2-ylmethyl]-L-tyrosine;
N-(2,6-dichlorobenzoyl)-O-[2-(methylamino) ethyl]-L-tyrosine;
N-(2,6-Dichlorobenzoyl)-O-(1-methylpiperidin-4-yl)-L-tyrosine;
N-(2,6-Dichlorobenzoyl)-O-[(3R)-1-methylpyrrolidin-3-yl]-L-tyrosine;
N-(2,6-Dichlorobenzoyl)-O-{[(2S)-1-methylpyrrolidin-2-yl]methyl}-L-tyrosine;
N-(2,6-Dichlorobenzoyl)-O-(1-acetylpiperidin-4-yl)-L-tyrosine;
N-(2,6-dichlorobenzoyl)-O-(1-glycoloylpiperidin-4-yl)-L-tyrosine;
N-(2,6-Dichlorobenzoyl)-O-[1-(N,N-dimethylglycyl)piperidin-4-yl]-L-tyrosine;
O-[(3R)-1-Acetylpyrrolidin-3-yl]-N-(2,6-dichlorobenzoyl)-L-tyrosine;
N-(2,6-Dichlorobenzoyl)-O-[(3R)-1-glycoloylpyrrolidin-3-yl]-L-tyrosine;
O-{3-[Acetyl(methyl)amino]propyl}-N-(2,6-dichlorobenzoyl)-L-tyrosine;
O-{[(2R)-1-Acetylpyrrolidin-2-yl]methyl}-N-(2,6-dichlorobenzoyl)-L-tyrosine;
N-(2,6-Dichlorobenzoyl)-O-{[(2S)-1-(N,N-dimethylglycyl)pyrrolidin-2-yl]methyl}-L-tyrosine;
N-(2,6-Dichlorobenzoyl)-O-{[(2S)-1-glycoloylpyrrolidin-2-yl]methyl}-L-tyrosine;
N-(2,6-dichlorobenzoyl)-O-[1-(methylsulfonyl)piperidin-4-yl]-L-tyrosine;
N-(2,6-Dichlorobenzoyl)-O-[(3R)-1-(methylsulfonyl)pyrrolidin-3-yl]-L-tyrosine;
N-(2,6-Dichlorobenzoyl)-O-{3-[methyl(methylsulfonyl)amino]propyl}-L-tyrosine;
N-(2,6-Dichlorobenzoyl)-O-{[(2S)-1-(methylsulfonyl)pyrrolidin-2-yl]methyl}-L-tyrosine;
N-(2,6-Dichlorobenzoyl)-O-{[(2R)-1-(methylsulfonyl)pyrrolidin-2-yl]methyl}-L-tyrosine;
N-(2,6-Dichlorobenzoyl)-O-{3-[methyl(methylsulfonyl)amino]ethyl}-L-tyrosine;
N-(2,6-Dichlorobenzoyl)-O-(1-phenylpiperidin-4-yl)-L-tyrosine;
N-(2,6-Dimethylbenzoyl)-O-(1-phenylpiperidin-4-yl)-L-tyrosine;
N-(2-Chloro-6-methylbenzoyl)-O-(1-phenylpiperidin-4-yl)-L-tyrosine;
N-(2-Chloro-4,5-dimethoxybenzoyl)-O-(1-phenylpiperidin-4-yl)-L-tyrosine;
N-(2,6-dichlorobenzoyl)-O-[1-(4-fluoro-3-methylbenzoyl)piperidin-4-yl]-L-tyrosine;
N-(2,6-dichlorobenzoyl)-O-[1-(4-fluoro-3-methylbenzoyl)piperidin-4-yl]-L-tyrosine;
O-[1-(2,1-benzisoxazol-3-ylcarbonyl)piperidin-4-yl]-N-(2,6-dichlorobenzoyl)-L-tyrosine;
N-(2,6-dichlorobenzoyl)-O-[1-(4-fluoro-3-methylbenzoyl)azetidin-3-yl]-L-tyrosine;
N-(2,6-dichlorobenzoyl)-O-[1-(quinolin-4-ylcarbonyl)azetidin-3-yl]-L-tyrosine;
N-(2,6-dichlorobenzoyl)-O-[(3S)-1-(4-fluoro-3-methylbenzoyl)pyrrolidin-3-yl]-L-tyrosine;
O-[(3S)-1-(2,1-benzisoxazol-3-ylcarbonyl)pyrrolidin-3-yl]-N-(2,6-dichlorobenzoyl)-L-tyrosine;
N-(2,6-dichlorobenzoyl)-O-{1-[(2,5-dimethyl-3-thienyl)carbonyl]piperidin-4-yl}-L-tyrosine;
N-(2,6-dichlorobenzoyl)-O-{(3R)-1-[(2,5-dimethyl-3-thienyl)carbonyl]pyrrolidin-3-yl}-L-tyrosine;
N-(2,6-dichlorobenzoyl)-O-[1-(4-fluorobenzyl)azetidine-3-yl]-L-tyrosine;
N-(2,6-dichlorobenzoyl)-O-[1-(4-fluorobenzyl)azetidin-3-yl]-L-tyrosine;
N-(2,6-dichlorobenzoyl)-O-[1-(1H-indol-5-ylmethyl)azetidin-3-yl]-L-tyrosine;
N-(2,6-dichlorobenzoyl)-O-[1-(4-(trifluoromethyl)benzyl)azetidin-3-yl]-L-tyrosine;
O-{1-[3-(aminocarbonyl)benzyl]azetidin-3-yl}-N-(2,6-dichlorobenzoyl)-L-tyrosine;
O-[1-(3-chloro-4-fluorobenzyl)azetidin-3-yl]-N-(2,6-dichlorobenzoyl)-L-tyrosine;
O-[1-(4-cyanobenzyl)azetidin-3-yl]-N-(2,6-dichlorobenzoyl)-L-tyrosine;
N-(2,6-dichlorobenzoyl)-O-{1-[4-(trifluoromethoxy)benzyl]azetidin-3-yl}-L-tyrosine;
O-[(3S)-1-benzylpyrrolidin-3-yl]-N-(2,6-dichlorobenzoyl)-L-tyrosine;
O-[(3S)-1-benzylpyrrolidin-3-yl]-N-(2,6-dichlorobenzoyl)-L-tyrosine;
N-(2,6-dichlorobenzoyl)-O-((3R)-1-{4-[(dimethylamino)carbonyl]benzyl}pyrrolidin-3-yl)-L-tyrosine;
N-(2,6-dichlorobenzoyl)-O-[(3S)-1-(4-fluorobenzyl)pyrrolidin-3-yl]-L-tyrosine;
N-(2,6-dichlorobenzoyl)-O-[(3S)-1-(4-methoxybenzyl)pyrrolidin-3-yl]-L-tyrosine;
N-(2,6-dichlorobenzoyl)-O-[(3S)-1-(4-methylbenzyl)pyrrolidin-3-yl]-L-tyrosine;
O-[(3R)-1-(4-cyanobenzyl)pyrrolidin-3-yl]-N-(2,6-dichlorobenzoyl)-L-tyrosine;
N-(2,6-dichlorobenzoyl)-O-{(3S)-1-[4-(methylsulfonyl)benzyl]pyrrolidin-3-yl}-L-tyrosine;
O-[(3R)-1-(3-chloro-4-fluorobenzyl)pyrrolidin-3-yl]-N-(2,6-dichlorobenzoyl)-L-tyrosine;
N-(2,6-dichlorobenzoyl)-O-[(3R)-1-(4-methylbenzyl)piperidin-3-yl]-L-tyrosine;
N-(2,6-dichlorobenzoyl)-O-[(3R)-1-(4-methylbenzyl)piperidin-3-yl]-L-tyrosine;
O-[(3S)-1-(4-chlorobenzyl)piperidin-3-yl]-N-(2,6-dichlorobenzoyl)-L-tyrosine;
O-[(3S)-1-benzylpiperidin-3-yl]-N-(2,6-dichlorobenzoyl)-L-tyrosine;
O-[(3S)-1-(4-cyanobenzyl)piperidin-3-yl]-N-(2,6-dichlorobenzoyl)-L-tyrosine;
N-(2,6-dichlorobenzoyl)-O-((3R)-1-{4-[(dimethylamino)carbonyl]benzyl}piperidin-3-yl)-L-tyrosine;
N-(2,6-dichlorobenzoyl)-O-[(3S)-1-(4-methoxybenzyl)piperidin-3-yl]-L-tyrosine;
N-(2,6-dichlorobenzoyl)-O-[(3S)-1-(2,5-difluorobenzyl)piperidin-3-yl]-L-tyrosine;
N-(2,6-dichlorobenzoyl)-O-{(3S)-1-[4-(methylsulfonyl)benzyl]piperidin-3-yl}-L-tyrosine;
N-(2,6-dichlorobenzoyl)-O-[1-(4-fluorobenzyl)piperidin-4-yl]-L-tyrosine;
N-(2,6-dichlorobenzoyl)-O-[1-(4-fluorobenzyl)piperidin-4-yl]-L-tyrosine;
N-(2,6-dichlorobenzoyl)-O-[1-(2-methoxybenzyl)piperidin-4-yl]-L-tyrosine;
O-[1-(3-chlorobenzyl)piperidin-4-yl]-N-(2,6-dichlorobenzoyl)-L-tyrosine;
N-(2,6-dichlorobenzoyl)-O-{1-[4-(trifluoromethoxy)benzyl]piperidin-4-yl}-L-tyrosine;
N-(2,6-dichlorobenzoyl)-O-[1-(2,4-difluorobenzyl)piperidin-4-yl]-L-tyrosine;
O-[1-(3-cyanobenzyl)piperidin-4-yl]-N-(2,6-dichlorobenzoyl)-L-tyrosine;
N-(2,6-dichlorobenzoyl)-O-[1-(quinolin-8-ylmethyl)piperidin-4-yl]-L-tyrosine;
N-(2,6-dichlorobenzoyl)-O-{1-[4-(methylsulfonyl)benzyl]piperidin-4-yl}-L-tyrosine; or
O-[1-(4-chloro-3-methoxybenzyl)piperidin-4-yl]-N-(2,6-dichlorobenzoyl)-L-tyrosine;
or a pharmaceutically acceptable salt thereof.

26. A pharmaceutical composition comprising a compound of claim 16 or a pharmaceutically acceptable salt thereof in association with a pharmaceutically acceptable carrier, diluent, or excipient.

27. A compound of formula I according to claim 16, which is an integrin inhibitor useful for controlling pathologically angiogenic diseases, thrombosis, cardiac infarction, coronary heart diseases, arteriosclerosis, tumors, osteoporosis, inflammations or infections.

28. A method of treating a disease or condition mediated by a5b1 which comprises administering to a patient in need of such treatment a compound of formula (I) as defined in claim 16 or a pharmaceutically acceptable salt thereof.

29. A method according to claim 28 in which the disease or condition is cancer.

30. A process for the preparation of a compound of formula (I) as defined in claim 16 which comprises any one of processes (a) to (h) as follows (wherein the variables are as defined above unless otherwise stated):
Process (a) coupling a compound of the formula (V): 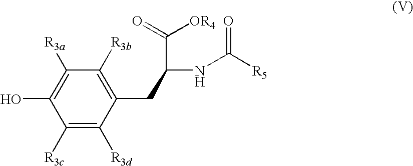
wherein R₃ₐ, R_{3b}, R_{3c}, R_{3d}, R₄ and R₅, are as defined in claim 16, except any functional group is protected if necessary,
with a compound of the formula (VI):
Q₁-OH VI
wherein Q₁ is as defined in claim 16, except any functional group is protected if necessary; or
Process (b) for the preparation of those compounds of formula I either (b′) R₁ is a group of the formula RₓS(O)₂— or (b″) R₈ is a C₁₋₆alkylsulphonyl group which may be optionally substituted as defined in claim 16 for R₈, the reaction, conveniently in the presence of a suitable base, of a compound of the formula I of the formula Ia: 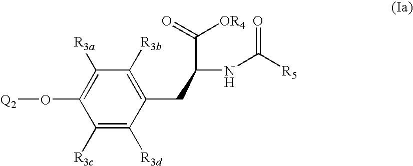
where Q₂ is a group of sub-formula (ai) or (bi) 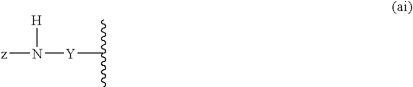
where Y and Z are as defined herein; 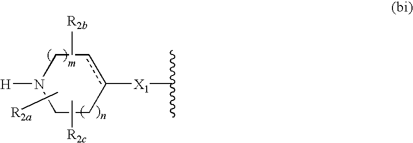
wherein X₁, R₂ₐ, R_{2b}, R_{2c}, m and n are as defined in claim 16, except any functional group is protected if necessary,
with a compound of the formula VII: 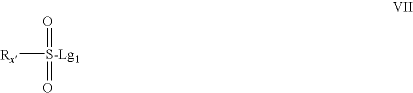
wherein in the case of process (b′) R_{x′}, is a group Rₓ is as defined in claim 16, or in the case of process (b″) R_{x′} an alkyl group optionally substituted by one or more more groups selected from the optional substituents listed above for R₈, except any functional group is protected if necessary,
and Lg₁ is a leaving group; or
Process (c) for the preparation of those compounds of formula I wherein, either (c′) R₁ is a group of the formula R_{y}C(O)— or (c″) R₈ is a (C₂-C₆)alkanoyl group optionally substituted as defined above, the coupling, conveniently in the presence of a suitable base of a compound of the formula I of the formula Ia as hereinbefore defined in relation to Process (b) with a compound of the formula VIII or a reactive derivative thereof:
R_{y′}COOH VIII
wherein in the case of process (c′), R_{y′} is a group R_{y} as defined in claim 16, or in the case of (c″) R_{y′} is a C₁₋₅alkyl group optionally substituted by one or more groups selected from the optional substitutents defined for R₈, provided that any reactive groups are optionally protected;
or
Process (d) for the preparation of those compounds of formula I wherein Q₁ is a group of sub-formula (bi) and “- - -” in the compounds of formula I is absent, the reduction of a compound of the formula I wherein “- - -” is a bond; or
Process (e) the coupling of a compound of the formula IX: 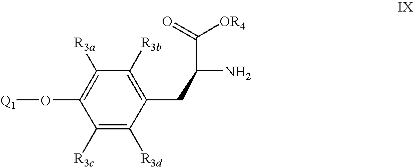
wherein Q₁, R₃ₐ, R_{3b}, R_{3c}, R_{3d}, R₄, X₁, m and n are as defined in claim 16, except any functional group is protected if necessary,
with a compound of the formula X or a reactive derivative thereof:
R₅COOH X
wherein R₅ is as defined in claim 16, except any functional group is protected if necessary; or
Process (f) for the preparation of those compounds of formula I wherein, either (f′) R₁ is optionally substituted (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aralkyl or heteroaralkyl, or (f″) R₈ is a (C₁-C₆)alkyl group, the reaction, conveniently in the presence of a suitable base, of a compound of the formula Ia as hereinbefore defined in relation to Process (b), with a compound of the formula XI:
R_{1′}-Lg₂ XI
wherein, in the case of (f″) R_{1′} is optionally substituted (C₁-C₆)alkyl, (C₃-C₆)cycloalkyl, heterocyclyl, (C₃-C₆)cycloalkyl(C₁-C₆)alkyl, heterocyclyl(C₁-C₆)alkyl, aralkyl or heteroaralkyl, or in the case of (f″) R_{1′} is (C₁-C₆)alkyl optionally substituted as defined above for R₈: and
Lg₂ is a suitable leaving group; or
Process (g) for the preparation of those compounds of formula I wherein R₁ or R₈ is a group of the formula R′HNC(O)—, the reaction of a compound of the formula Ia as hereinbefore defined in relation to Process (b) with an isocyanate of the formula XII:
R′N═C(O) XII
wherein R′ is as hereinbefore defined, except any functional group is protected if necessary; or
Process (h) for the preparation of those compounds of formula I wherein R₁ or R₈ is aryl or heteroaryl, the coupling in the presence of a suitable catalyst, of a compound of the formula I of the formula Ia as hereinbefore defined in relation to Process (b) with an aryl or heteroaryl boronic acid, or an ester thereof;
and thereafter, if necessary (in any order):
(i) converting a compound of the formula I into another compound of the formula I;
(ii) removing any protecting groups; and
(iii) forming a pharmaceutically acceptable salt of the compound of formula I.
